(19) United States
(12) Patent Application Publication
(10) Pub. No.: US 2023/0303705 A1
(43) Pub. Date: Sep. 28, 2023

(54) BINDERS AND CHIMERIC ANTIGEN RECEPTORS SPECIFIC FOR INTERLEUKIN-1 RECEPTOR ACCESSORY PROTEIN

(71) Applicants: Seattle Children's Hospital (dba Seattle Children's Research Institute), Seattle, WA (US); Provincial Health Services Authority, Vancouver (CA); University of Pittsburgh - of the Commonwealth System of Higher Education, Pittsburgh, PA (US)

(72) Inventors: Rimas J. Orentas, Seattle, WA (US); Dimiter Stanchev Dimitrov, Pittsburgh, PA (US); Poul H. Sorensen, Vancouver (CA); Haifeng Zhang, Vancouver (CA); Wei Li, Pittsburgh, PA (US)

(21) Appl. No.: 17/917,113

(22) PCT Filed: Apr. 9, 2021
(86) PCT No.: PCT/US2021/026727

§ 371 Date: Oct. 5, 2022

Related U.S. Application Data
(60) Provisional application No. 63/008,173, filed on Apr. 10, 2020.

(51) Int. Cl. C07K 16/28

(52) U.S. Cl. CPC C07K 16/2866; C07K 2319/02; C07K 2319/03

(57) ABSTRACT

Some embodiments provided herein also include methods and materials involved in binding a binder (e.g., an antibody, antigen binding fragment, antibody domain, CAR, cell engager, and/or ADC) to an IL1RAP polypeptide. For example, binders (e.g., antibodies, antigen binding fragments, antibody domains, CARs, cell engagers, and/or ADCs) that bind to an IL1RAP polypeptide and methods and materials for using one or more such binding molecules to treat a mammal (e.g., a human) having cancer are provided. Some embodiments of the methods and compositions provided herein include chimeric antigen receptors (CARs) which specifically bind to interleukin-1 receptor accessory protein (IL1RAP). Some embodiments include nucleic acids encoding such CARs, and cells containing such CARs. Some embodiments include the use of such CARs in safe and effective therapies for a cancer, such as an IL1RAP-expressing cancer, such as a Ewing's sarcoma and acute myeloid leukemia (AML).

### CROSS-REFERENCE TO RELATED APPLICATIONS

This application is a U.S. National Phase Application of PCT International Application Number PCT/US2021/026727, filed on Apr. 9, 2021, designating the United States of America and published in the English language, which is an International Application of and claims the benefit of priority to U.S. Provisional Application No. 63/008,173, filed on Apr. 10, 2020. The disclosures of the above-referenced applications are hereby expressly incorporated by reference in their entireties.

### REFERENCE TO SEQUENCE LISTING

The present application is being filed along with a Sequence Listing in electronic format. The Sequence Listing is provided as a file entitled SCRI282NPSEQLIST, created Feb. 17, 2023, which is approximately 194,171 bytes in size. The information in the electronic format of the Sequence Listing is incorporated herein by reference in its entirety.

### FIELD

Embodiments provided herein relate to methods and materials involved in binding a molecule (e.g., an antibody, a fragment of an antibody, an antibody domain, a chimeric antigen receptor (CAR), a cell engager, or an antibody-drug conjugate (ADC)) to interleukin-1 receptor accessory protein (IL1RAP). For example, some embodiments include binders (e.g., antibodies, antigen binding fragments, antibody domains, CARs, cell engagers, or ADCs) that bind to an IL1RAP polypeptide and methods and materials for using such binders to treat cancer. Some embodiments include cells (e.g., host cells) designed to express one or more binders (e.g., antibodies, antigen binding fragments, antibody domains, CARs, or cell engagers) having the ability to bind to an IL1RAP polypeptide and methods and materials for using such cells to treat cancer.

Aspects of the methods and compositions provided herein include chimeric antigen receptors (CARs), which specifically bind to interleukin-1 receptor accessory protein (IL1RAP). Some alternatives include nucleic acids encoding these CARs, and cells containing such CARs. Some alternatives also include the use of these CARs in safe and effective therapies for a cancer, such as an IL1RAP-expressing cancer, preferably a Ewing's sarcoma and acute myeloid leukemia (AML).

### BACKGROUND

The interleukin-1 (IL-1) family of cytokine ligands and receptors is associated with inflammation, autoimmunity, immune regulation, cell proliferation, and host defense and contributes to the pathology of inflammatory, autoimmune, immune regulatory, degenerative, and cell proliferative diseases and disorders, and its cytokines and receptors serve as pathogenic mediators of such diseases and disorders.

The IL-1 family of cytokines includes IL-1α, IL-1β, IL-33, IL-36α, IL-36 β, and IL-36γ. Each of these cytokines serves as a ligand capable of binding a specific IL-1 family cell membrane receptor expressed on the surface of certain cells. Upon binding of an IL-1 family cytokine to its cognate receptor, a co-receptor is recruited to form a ternary complex comprising the cytokine, its cognate membrane receptor, and its co-receptor. The resulting ternary complex facilitates intracellular signal transduction and activation of a set of transcription factors, including NFκB and AP-1, and mitogen-activated protein kinases, which triggers a cascade of inflammatory and immune responses, including the production of numerous cytokines, chemokines, enzymes, and adhesion molecules.

Interleukin-1 receptor accessory protein (IL1RAP) serves as the common cellular membrane co-receptor for several receptors in the IL-1 family, including interleukin-1 receptor 1 (IL1R1), ST2, and interleukin-1 receptor-like 2 (IL1RL2). IL1RAP is a necessary component of the ternary signaling complex formed by one of the IL-1 family cytokines, the cytokine's specific cognate receptor, and the IL1RAP co-receptor. Thus, IL1RAP serves an important function in the IL-1 family signal transduction pathways, since it is required to facilitate particular downstream signaling pathways stimulated by the IL-1 family cytokines IL-1α, IL-1β, IL-33, IL-36α, IL-36 β, and IL-36γ. Therefore, there remains a need for safe and effective therapies to treat, ameliorate, inhibit, or prevent inflammatory, autoimmune, immune regulatory, degenerative, and cell proliferative diseases or disorders associated with the IL-1 family of cytokine ligands and receptors, and in particular, IL1RAP.

### SUMMARY

Embodiments provided herein relate to methods and materials involved in binding a molecule (e.g., an antibody, an antigen binding fragment, an antibody domain, a CAR, a cell engager, or an ADC) to an IL1RAP polypeptide. For example, some embodiments include binders (e.g., antibodies, antigen binding fragments, antibody domains, CARs, cell engagers, or ADCs) that bind to an IL1RAP polypeptide and methods and materials for using one or more such binders to treat a mammal (e.g., a human) having cancer.

Some embodiments provided herein also include cells (e.g., host cells) designed to express one or more binders (e.g., antibodies, antigen binding fragments, antibody domains, CARs, or cell engagers) having the ability to bind to an IL1RAP polypeptide and methods and materials for using such cells to treat cancer.

As described herein, binders (e.g., one or more antibodies, one or more antigen binding fragments, one or more antibody domains, one or more CARs, one or more cell engagers, and/or one or more ADCs) can be designed to have the ability to bind to an IL1RAP polypeptide. For example, a binder (e.g., an antibody, an antigen binding fragment, an antibody domain, a CAR, a cell engager, or an ADC) provided herein can have the ability to bind to a polypeptide comprising, consisting essentially of, or consisting of the amino acid sequence of a human IL1RAP polypeptide as set forth in SEQ ID NO:54 or SEQ ID NO:55 (see, e.g., FIG. 6).

In some cases, a single set of three complementarity-determining regions (CDRs) of an antibody domain (e.g., a VH domain) provided herein (e.g., SEQ ID NOs:42-44; SEQ ID NOs:196-198; SEQ ID NOs:45-47; SEQ ID NOs:203-205; SEQ ID NOs:48-50; SEQ ID NOs:210-212; SEQ ID NOs:51-53; SEQ ID NOs:217-219; SEQ ID NOs:161-163; SEQ ID NOs:169-171; SEQ ID NOs:177-179; or SEQ ID NOs:185-187) can be engineered into a CAR to create CAR⁺ cells (e.g., CAR⁺ T cells, CAR⁺ stem cells such as CAR⁺ induced pluripotent stem cells, or CAR⁺ natural killer (NK) cells) having the ability to target IL1RAP⁺ cells (e.g., IL1RAP⁺ tumor cells and/or IL1RAP⁺ tumor vasculature), can be engineered into an antibody structure that includes an Fc region to create antibodies having the ability to target IL1RAP⁺ cells (e.g., IL1RAP⁺ tumor cells and/or IL1RAP⁺ tumor vasculature) and induce antibody-dependent cell-mediated cytotoxicity (ADCC) against the target IL1RAP⁺ cells, and/or can be engineered into a cell engager such as a bi-specific T cell engager (e.g., a BiTE), a bi-specific killer engager (e.g., a BiKE), and/or a tri-specific killer engager (e.g., a TriKE) to create cell engagers having the ability to target IL1RAP⁺ cells (e.g., IL1RAP⁺ tumor cells and/or IL1RAP⁺ tumor vasculature) and induce one or more immune responses (e.g., T cell immune responses and/or ADCC using a cell engager in the absence of an Fc-containing antibody) against the target IL1RAP⁺ cells. It is noted that BiKE- and TriKE-mediated killing can be referred to ADCC even though it is not initiated by an Fc domain.

In addition, as described herein, binders (e.g., one or more antibodies, one or more antigen binding fragments, and/or one or more antibody domains) provided herein can be used to create conjugates that include the binder and a drug. For example, ADCs such as full antibody-drug conjugates, Fab-drug conjugates, and/or antibody domain-drug conjugates can be designed to include an appropriate binder provided herein to create the conjugate. Such conjugates can be used to deliver the drug payload to target cells such as cancer cells (e.g., IL1RAP⁺ cancer cells) or cancer vasculature (e.g., IL1RAP⁺ cancer vasculature).

As also described herein, binders (e.g., one or more antibodies, one or more antigen binding fragments, one or more antibody domains, one or more cell engagers, and/or one or more ADCs) provided herein can be used to treat a mammal (e.g., a human) having cancer. For example, a mammal (e.g., a human) having cancer (e.g., a IL1RAP⁺ cancer) can be administered a composition comprising one or more binders (e.g., one or more antibodies, one or more antigen binding fragments, one or more antibody domains, one or more cell engagers, and/or one or more ADCs) described herein to reduce the number of cancer cells within the mammal, to induce ADCC against cancer cells within the mammal, and/or to increase the survival duration of the mammal from cancer.

As also described herein, cells (e.g., host cells) can be designed to express one or more binders (e.g., antibodies, antigen binding fragments, antibody domains, CARs, or cell engagers) having the ability to bind to an IL1RAP polypeptide. For example, cells such as T cells (e.g., CTLs), stem cells (e.g., induced pluripotent stem cells), or NK cells can be engineered to express one or more CARs having the ability to bind to an IL1RAP polypeptide. Such cells (e.g., IL1RAP-specific CAR⁺ T cells or NK cells) can be used to treat cancer.

In some cases, a binder (e.g., an antibody, antigen binding fragment, and/or antibody domain) provided herein can be used to detect the presence or absence of an IL1RAP polypeptide. For example, a binder (e.g., an antibody, antigen binding fragment, and/or antibody domain) provided herein can be used to determine whether or not a sample (e.g., a biological sample such tumor biopsy) obtained from a mammal (e.g., a human) contains IL1RAP⁺ cells (e.g., IL1RAP⁺ cancer cells). Having the ability to detect the presence or absence of an IL1RAP polypeptide (e.g., IL1RAP⁺ cancer cells) can allow clinicians, health professionals, and patients to make better decisions about possible treatment options. For example, detection of IL1RAP⁺ cancer cells within a mammal can allow clinicians, health professionals, and patients to select an appropriate anti-cancer treatment that targets the IL1RAP⁺ cancer cells. Such treatments that target the IL1RAP⁺ cancer cells can include administration of an anti-IL1RAP antibody such as CANO4 (nidanilimab) and/or one or more of the binders described herein having the ability to bind to an IL1RAP polypeptide and/or administration of one or more cells (e.g., IL1RAP-specific CAR⁺ T cells or NK cells) designed to express a binder described herein.

Some alternatives include a nucleic acid encoding a chimeric antigen receptor (CAR), wherein the CAR comprises: a ligand binding domain, which specifically binds to an interleukin-1 receptor accessory protein (IL1RAP); a spacer; a transmembrane domain; and an intracellular signalling domain.

In some alternatives, the ligand binding domain of the CAR encoded by the nucleic acid comprises a complementarity-determining region (CDR) comprising an amino acid sequence having 0-4 conservative amino acid substitutions of any one of SEQ ID NOs:42-53. In some alternatives, the ligand binding domain of the CAR encoded by the nucleic acid comprises a complementarity-determining region (CDR) comprising an amino acid sequence having 0-4 conservative amino acid substitutions of any one of SEQ ID NOs:42-44, or 51-53. In some alternatives, the ligand binding domain of the CAR encoded by the nucleic acid comprises a complementarity-determining region 3 (CDR3) comprising an amino acid sequence having 0-4 conservative amino acid substitutions of SEQ ID NO:44 or SEQ ID NO:53.

In some alternatives, the ligand binding domain of the CAR encoded by the nucleic acid comprises: a complementarity-determining region 1 (CDR1) comprising an amino acid sequence having 0-4 conservative amino acid substitutions of SEQ ID NO:51; a complementarity-determining region 2 (CDR2) comprising an amino acid sequence having 0-4 conservative amino acid substitutions of SEQ ID NO:52; and a complementarity-determining region 3 (CDR3) comprising an amino acid sequence having 0-4 conservative amino acid substitutions of SEQ ID NO:44 or SEQ ID NO:53. In some alternatives, the ligand binding domain of the CAR encoded by the nucleic acid comprises the amino acid sequences SEQ ID NO:51, SEQ ID NO:52, and SEQ ID NO:53.

In some alternatives, the ligand binding domain of the CAR encoded by the nucleic acid comprises an amino acid sequence having at least 95% identity to the amino acid sequence of any one of SEQ ID NOs:01-04. In some alternatives, the ligand binding domain of the CAR comprises the amino acid sequence of SEQ ID NO:01 or SEQ ID NO:04.

In some alternatives, the spacer of the CAR encoded by the nucleic acid comprises a CD8 spacer domain or an IgG4 hinge region.

In some alternatives, the transmembrane domain of the CAR encoded by the nucleic acid comprises a CD8 transmembrane domain. In some alternatives, the transmembrane domain of the CAR encoded by the nucleic acid comprises an amino acid sequence having at least 95% identity to the amino acid sequence of SEQ ID NO:13. In some alternatives, the transmembrane domain of the CAR encoded by the nucleic acid comprises the amino acid sequence of SEQ ID NO:13.

In some alternatives, the intracellular signalling domain of the CAR encoded by the nucleic acid comprises a costimulatory domain selected from the group consisting of CD27, CD28, 4-1BB, OX-40, CD30, CD40, PD-1, ICOS, LFA-1, CD2, CD7, NKG2C, and B7-H3, in combination with a CD3zeta domain or functional portion thereof. In some alternatives, the intracellular signalling domain of the CAR encoded by the nucleic acid comprises a 4-1BB costimulatory domain. In some alternatives, the 4-1BB costimulatory domain comprises an amino acid sequence having at least 95% identity to the amino acid sequence of SEQ ID NO:15. In some alternatives, the 4-1BB costimulatory domain comprises the amino acid sequence of SEQ ID NO:15. In some alternatives, the CD3 zeta domain or functional portion thereof comprises an amino acid sequence having at least 95% identity to the amino acid sequence of SEQ ID NO:17. In some alternatives, the CD3 zeta domain or functional portion thereof comprises the amino acid sequence of SEQ ID NO:17.

Some alternatives also include a polynucleotide encoding a selectable marker. In some alternatives, the selectable marker comprises a cell surface selectable marker selected from a truncated EGFR polypeptide (EGFRt) or a truncated HER2 polypeptide (HER2t).

Some alternatives include a polypeptide encoded by the nucleic acid of any one of the foregoing alternatives.

Some alternatives include a vector comprising the nucleic acid of any one of the foregoing alternatives.

In some alternatives, the vector comprises a viral vector.

In some alternatives, the viral vector comprises a lentiviral vector, a retroviral vector, an adenoviral vector, or an adenovirus associated viral vector.

Some alternatives include a cell comprising the nucleic acid of any one of the foregoing alternatives.

In some alternatives, the cell is a CD4+ T-cell or a CD8+ T-cell.

In some alternatives, the cell is a precursor T-cell, or a hematopoietic stem cell.

In some alternatives, the cell is a CD8+ cytotoxic T-cell selected from the group consisting of a naïve CD8+ T-cell, a CD8+ memory T-cell, a central memory CD8+ T-cell, a regulatory CD8+ T-cell, an IPS derived CD8+ T-cell, an effector memory CD8+ T-cell, and a bulk CD8+ T-cell.

In some alternatives, the cell is a CD4+T helper cell selected from the group consisting of a naïve CD4+ T-cell, a CD4+ memory T-cell, a central memory CD4+ T-cell, a regulatory CD4+ T-cell, an IPS derived CD4+ T-cell, an effector memory CD4+ T-cell, and a bulk CD4+ T-cell.

Some alternatives include the cell of any one of the foregoing alternatives for use in treating, ameliorating or inhibiting a cancer in a subject.

In some alternatives, the cancer is selected from the group consisting of a breast cancer, a brain cancer, a colon cancer, a renal cancer, a pancreatic cancer, an ovarian cancer, a sarcoma, and a leukemia.

In some alternatives, the cancer comprises an acute myeloid leukemia (AML), a chronic myelogenous leukemia (CML), or a Ewing's sarcoma.

Some alternatives include a pharmaceutical composition comprising the cell of any one of the foregoing alternatives and a pharmaceutically acceptable excipient.

Some alternatives include a method of treating, ameliorating or inhibiting a cancer in a subject, comprising administering the cell of any one of the foregoing alternatives to the subject.

In some alternatives, the cancer is selected from the group consisting of a breast cancer, a brain cancer, a colon cancer, a renal cancer, a pancreatic cancer, an ovarian cancer, a sarcoma, and a leukemia.

In some alternatives, the cancer comprises an acute myeloid leukemia (AML), a chronic myelogenous leukemia (CML), or a Ewing's sarcoma.

In some alternatives, the subject is mammalian.

In some alternatives, the subject is human.

Some alternatives include a method for preparing a population of cells, comprising: (a) introducing the nucleic acid of any one of the foregoing alternatives into an isolated population of cells; and (b) culturing the population of cells in the presence of an agent selected from an anti-CD3, an anti-CD28, and a cytokine such as IL-2.

In some alternatives, the population of cells comprises a CD4+ T-cell or a CD8+ T-cell.

In some alternatives, the population of cells comprises a precursor T-cell, or a hematopoietic stem cell.

In some alternatives, the population of cells comprises a CD8+ cytotoxic T-cell selected from the group consisting of a naïve CD8+ T-cell, a CD8+ memory T-cell, a central memory CD8+ T-cell, a regulatory CD8+ T-cell, an IPS derived CD8+ T-cell, an effector memory CD8+ T-cell, and a bulk CD8+ T-cell.

In some alternatives, the population of cells comprises a CD4+T helper cell selected from the group consisting of a naïve CD4+ T-cell, a CD4+ memory T-cell, a central memory CD4+ T-cell, a regulatory CD4+ T-cell, an IPS derived CD4+ T-cell, an effector memory CD4+ T-cell, and a bulk CD4+ T-cell.

### BRIEF DESCRIPTION OF THE DRAWINGS

FIG. 1 depicts an example structure of a nucleic acid encoding a CAR and includes: lentiviral vector elements including a 5′ LTR and a 3′ LTR; a promoter, such as an E1α promoter; and polynucleotides encoding a ligand binding domain (binder), a spacer (linker), a CD8 transmembrane domain, a 4-1BB domain, and a CD3-zeta domain.

FIG. 2 depicts a FACS analysis for streptavidin-linked-phycoerythrin binding to cells expressing CARs and contacted with biotinylated IL1RAP.

FIG. 3 depicts a graph for specific lysis of Ewing's sarcoma TC71 target cells, which expressed IL1RAP, contacted with cells containing anti-IL1RAP CARs.

FIG. 4 depicts bar graphs of levels of cytokine production by effector (E) anti-IL1RAP CAR T cells co-cultured with target (T) Ewing sarcoma tumor cell lines expressing IL1RAP at various ratios (E:T). Top graph depicts levels of TNF-α expressed by co-cultured cells; middle panel depicts levels of IL-2 expressed by co-cultured cells; and lower panel depicts levels of IFN-γ expressed by co-cultured cells.

FIG. 5 depicts line graphs of levels of percentage lysis of target (T) AML cells co-cultured with effector (E) anti-IL1RAP CART cells at various ratios (E:T). Top graph depicts levels of lysis of co-cultured THP-1 cells; middle graph depicts levels of lysis of co-cultured MOLM-14 cells; and bottom panel depicts levels of lysis of co-cultured Raji cells.

FIG. 6 depicts amino acid residues 1 to 570 of a human IL1RAP polypeptide (SEQ ID NO:54). The bolded amino acid sequence (residues 21 to 367) of this human IL1RAP polypeptide depicts an extracellular domain (SEQ ID NO:55).

FIG. 7A depicts the amino acid sequence of a VH domain designated Clone 3A7 with alternative CDRs and framework sequences delineated. FIG. 7B depicts the amino acid sequence of a VH domain designated Clone 3A7 with the CDRs and framework sequences delineated.

FIG. 8A depicts the amino acid sequence of a VH domain designated Clone 4G6 with alternative CDRs and framework sequences delineated. FIG. 8B depicts the amino acid sequence of a VH domain designated Clone 4G6 with the CDRs and framework sequences delineated.

FIG. 9A depicts the amino acid sequence of a VH domain designated Clone 3C5 with alternative CDRs and framework sequences delineated. FIG. 9B depicts the amino acid sequence of a VH domain designated Clone 3C5 with the CDRs and framework sequences delineated.

FIG. 10A depicts the amino acid sequence of a VH domain designated Clone 7D12 with alternative CDRs and framework sequences delineated. FIG. 10B depicts the amino acid sequence of a VH domain designated Clone 7D12 with the CDRs and framework sequences delineated.

FIG. 11 depicts exemplary linker amino acid sequences for scFv's, CARs, and/or cell engagers.

FIGS. 12A and 12B depict the amino acid sequences of an exemplary heavy chain variable domain (FIG. 12A) and an exemplary light chain variable domain (FIG. 12B) of an exemplary scFv. The CDRs and framework sequences of each also are delineated. An exemplary linker amino acid sequence such as a linker amino acid sequence set forth in FIG. 11 can be used to link the heavy chain variable domain and the light chain variable domain together to form a scFv.

FIGS. 13A and 13B depict the amino acid sequences of an exemplary heavy chain variable domain (FIG. 13A) and an exemplary light chain variable domain (FIG. 13B) of an exemplary scFv. The CDRs and framework sequences of each also are delineated. An exemplary linker amino acid sequence such as a linker amino acid sequence set forth in FIG. 11 can be used to link the heavy chain variable domain and the light chain variable domain together to form a scFv.

FIG. 14 depicts the amino acid sequences of exemplary hinges that can be used to design a CAR.

FIG. 15 depicts the amino acid sequences of exemplary transmembrane domains that can be used to design a CAR.

FIG. 16 depicts the amino acid sequences of exemplary intracellular signaling domains that can be used to design a CAR.

FIG. 17 depicts the amino acid sequences of exemplary antigen binding domains that can be used to design cell engagers that bind to T cells.

FIG. 18 depicts the amino acid sequences of exemplary antigen binding domains that can be used to design cell engagers that bind to NK cells.

FIG. 19A is a schematic of an exemplary BiTE designed using CDR1, CDR2, and CDR3 of a VH domain provided herein. A humanized anti-CD3 scFv (e.g., an gOKT3-7 scFv set forth in U.S. Pat. No. 6,750,325) can be linked to the C-terminus of the VH domain via a linker (e.g., a (G4S)₃ linker). FIG. 19B depicts an amino acid of a linker sequence (SEQ ID NO:74) followed by an gOKT3-7 scFv sequence, which can be attached to a VH domain as shown in FIG. 19A. FIG. 19B also depicts a nucleic acid sequence encoding that linker and gOKT3-7 scFv.

FIG. 20 depicts the amino acid sequence of a VH domain designated Clone 7C1. The CDRs and framework sequences also are delineated.

FIG. 21 depicts the amino acid sequence of a VH domain designated Clone 7H2. The CDRs and framework sequences also are delineated.

FIG. 22 depicts the amino acid sequence of a VH domain designated Clone 6A2. The CDRs and framework sequences also are delineated.

FIG. 23 depicts the amino acid sequence of a VH domain designated Clone 6C1. The CDRs and framework sequences also are delineated.

FIG. 24 depicts the nucleic acid sequences encoding Clones 7C1, 7H2, 6A2, and 6C1.

FIG. 25 contains bar graphs plotting binding intensities to wild-type or IL1RAP knockout cells for the indicated clones.

### DETAILED DESCRIPTION

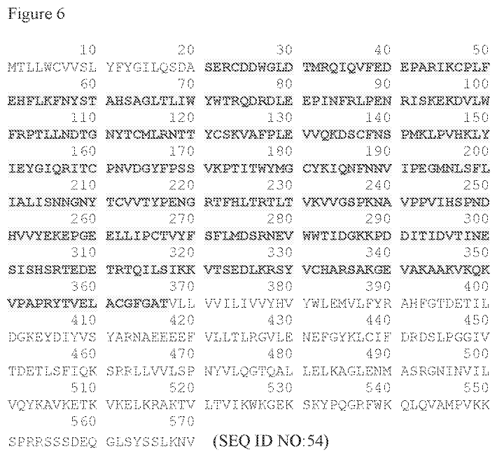

Embodiments provided herein relate to binders (e.g., antibodies, antigen binding fragments, antibody domains, CARs, cell engagers, and ADCs) that bind (e.g., specifically bind) to an IL1RAP polypeptide (e.g., a human IL1RAP polypeptide). For example, some embodiments include binders (e.g., antibodies, antigen binding fragments, antibody domains, CARs, cell engagers, and ADCs) that bind (e.g., specifically bind) to a polypeptide comprising, consisting essentially of, or consisting of the amino acid set forth in SEQ ID NO:54 or SEQ ID NO:55 (see, e.g., FIG. 6). In some cases, a binder (e.g., an antibody, an antigen binding fragment, an antibody domain, a CAR, a cell engager, or an ADC) provided herein can have the ability to bind to an IL1RAP polypeptide. For example, a binder (e.g., an antibody, an antigen binding fragment, an antibody domain, a CAR, a cell engager, or an ADC) provided herein can have the ability to bind to a human IL1RAP polypeptide.

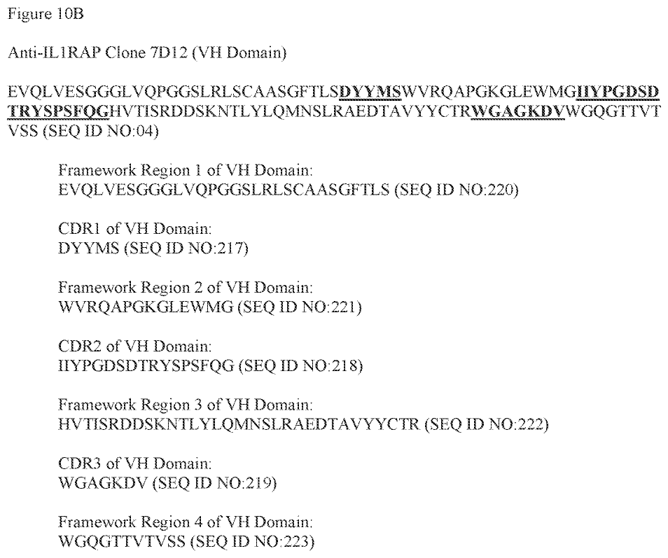

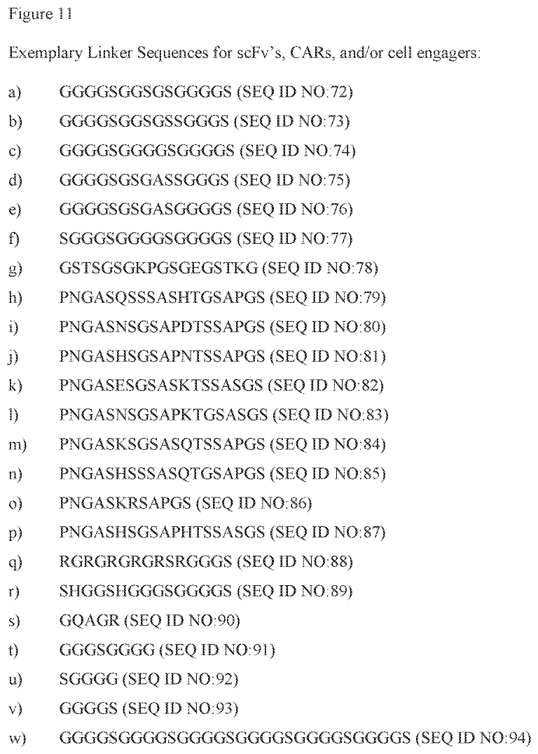

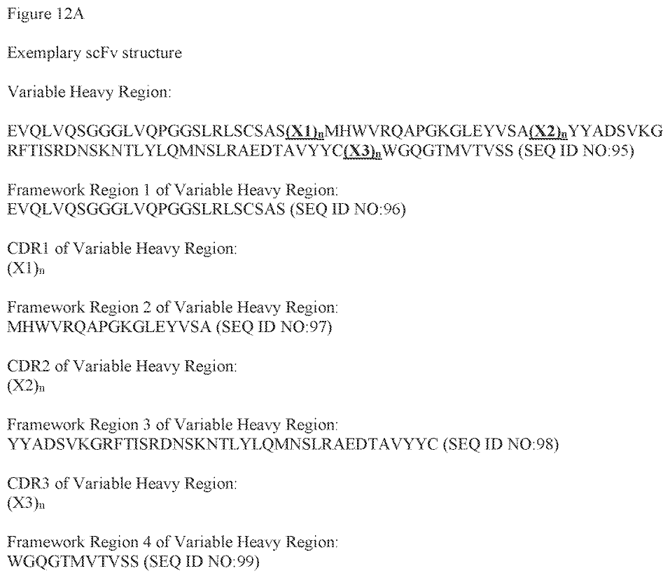

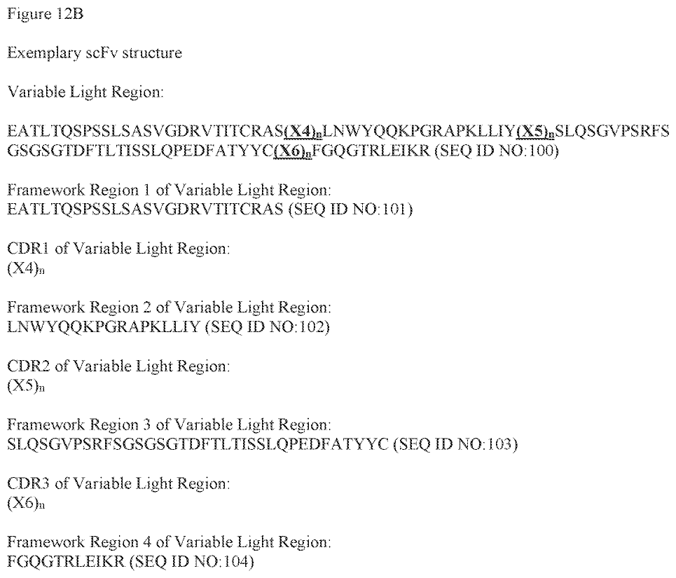

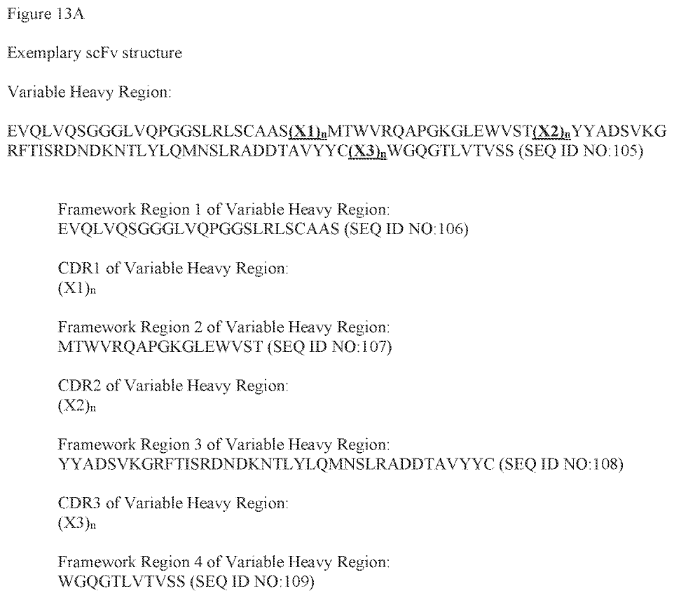

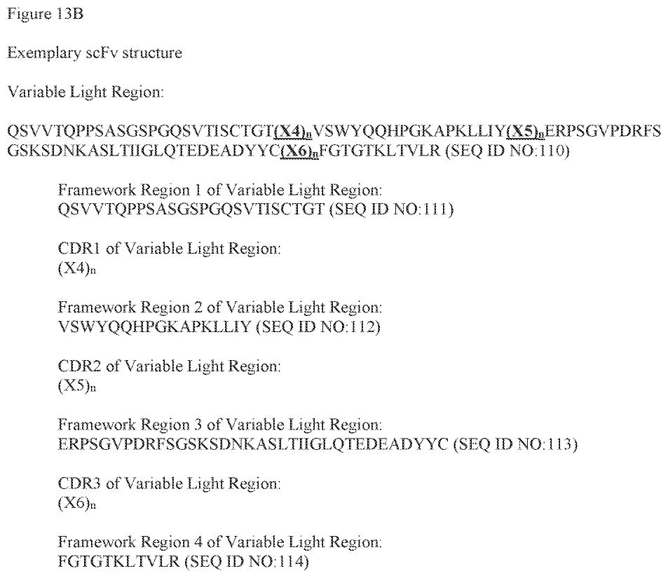

The term “antibody” as used herein includes polyclonal antibodies, monoclonal antibodies, recombinant antibodies, humanized antibodies, human antibodies, chimeric antibodies, multi-specific antibodies (e.g., bispecific antibodies) formed from at least two antibodies, diabodies, single-chain variable fragment antibodies (e.g., scFv antibodies), and tandem single-chain variable fragments antibody (e.g., taFv). A diabody can include two chains, each having a heavy chain variable domain and a light chain variable domain, either from the same or from different antibodies (see, e.g., Hornig and Farber-Schwarz, Methods Mol. Biol., 907:713-27 (2012); and Brinkmann and Kontermann, MAbs., 9(2):182-212 (2017)). The two variable regions can be connected by a polypeptide linker (e.g., a polypeptide linker having five to ten residues in length or a polypeptide linker as set forth in FIG. 11). In some cases, an interdomain disulfide bond can be present in one or both of the heavy chain variable domain and light chain variable domain pairs of the diabody. A scFv is a single-chain polypeptide antibody in which the heavy chain variable domain and the light chain variable domain are directly connected or connected via a polypeptide linker (e.g., a polypeptide linker having eight to 18 residues in length or a polypeptide linker as set forth in FIG. 11). See, also, Chen et al., Adv. Drug Deliv. Rev., 65(10):1357-1369 (2013). A scFv can be designed to have an orientation with the heavy chain variable domain being followed by the light chain variable domain or can be designed to have an orientation with the light chain variable domain being followed by the heavy chain variable domain. In both cases, the optional linker can be located between the two domains.

An antibody provided herein can include the CDRs as described herein (e.g., as described in Table 15) and can be configured to be a human antibody, a humanized antibody, or a chimeric antibody. In some cases, an antibody provided herein can include the CDRs as described herein (e.g., as described in Table 15) and can be a monoclonal antibody. In some cases, an antibody provided herein can include the CDRs as described herein (e.g., as described in Table 15) and can be configured as a scFv antibody.

The term “antigen binding fragment” as used herein refers to a fragment of an antibody (e.g., a fragment of a humanized antibody, a fragment of a human antibody, or a fragment of a chimeric antibody) having the ability to bind to an antigen. Examples of antigen binding fragments include, without limitation, Fab, Fab′, or F(ab′)2 antigen binding fragments. An antigen binding fragment provided herein can include the CDRs as described herein (e.g., as described in Table 15) and can be configured to be a human antigen binding fragment, a humanized antigen binding fragment, or a chimeric antigen binding fragment. In some cases, an antigen binding fragment provided herein can include the CDRs as described herein (e.g., as described in Table 15) and can be a monoclonal antigen binding fragment. In some cases, an antigen binding fragment provided herein can include the CDRs as described herein (e.g., as described in Table 15) and can be configured as an Fab antibody.

The term “antibody domain” as used herein refers to a domain of an antibody such as a heavy chain variable domain (VH domain) or a light chain variable domain (VL domain) in the absence of one or more other domains of an antibody. In some cases, an antibody domain can be a single antibody domain (e.g., a VH domain or a VL domain) having the ability to bind to an antigen. An antibody domain provided herein can include the CDRs as described herein (e.g., as described in Table 15) and can be a human antibody domain (e.g., a human VH domain), a humanized antibody domain (e.g., a humanized VH domain), or a chimeric antibody domain (e.g., a chimeric VH domain). In some cases, an antibody domain provided herein can include the CDRs as described herein (e.g., as described in Table 15) and can be a monoclonal antibody domain. In some cases, an antibody domain provided herein can include the CDRs as described herein (e.g., as described in Table 15) and can be engineered as a single VH domain or a single VL domain.

An anti-IL1RAP antibody, anti-IL1RAP antigen binding fragment, or anti-IL1RAP antibody domain provided herein can be of the IgA-, IgD-, IgE-, IgG-, or IgM-type, including IgG- or IgM-types such as, without limitation, IgG₁-, IgG₂-, IgG₃-, IgG₄-, IgM₁-, and IgM₂-types. In some cases, an antibody provided herein (e.g., an anti-IL1RAP antibody) can be a scFv antibody. In some cases, an antigen binding fragment provided herein (e.g., an anti-IL1RAP antibody fragment) can be an Fab. In some cases, an antibody provided herein (e.g., an anti-IL1RAP antibody) can be a fully intact antibody consisting of both VH and VL. In some cases, an antibody domain provided herein (e.g., an anti-IL1RAP antibody domain) can be a VH domain.

As described herein, a chimeric antigen receptor can refer to a chimeric polypeptide that is designed to include an antigen binding domain, an optional hinge, a transmembrane domain, and one or more intracellular signaling domains. As described herein, the antigen binding domain of a CAR provided herein can be designed to bind to an IL1RAP polypeptide (e.g., a human IL1RAP polypeptide). For example, a CAR provided herein can be designed to include the components of an antibody, antigen binding fragment, and/or antibody domain described herein (e.g., a combination of CDRs) as an antigen binding domain provided that that antigen binding domain has the ability to bind to an IL1RAP polypeptide (e.g., a human IL1RAP polypeptide). In some examples, a CAR provided herein can be designed to include an antigen binding domain that includes a single set of three CDRs (e.g., a CDR1, CDR2, and CDR3) of an antibody domain (e.g., a VH domain) provided herein (e.g., SEQ ID NOs:42-44; SEQ ID NOs:196-198; SEQ ID NOs:45-47; SEQ ID NOs:203-205; SEQ ID NOs:48-50; SEQ ID NOs:210-212; SEQ ID NOs:51-53; SEQ ID NOs:217-219; SEQ ID NOs:161-163; SEQ ID NOs:169-171; SEQ ID NOs:177-179; or SEQ ID NOs:185-187). In some cases, an antigen binding domain of a CAR targeting an IL1RAP polypeptide can be designed to include a VH domain described herein or a scFv antibody described herein.

In some cases, a CAR provided herein can be designed to include a hinge. Any appropriate hinge can be used to design a CAR described herein. Examples of hinges that can be used to make a CAR described herein include, without limitation, Ig-derived hinges (e.g., an IgG1-derived hinge, an IgG2-derived hinge, or an IgG4-derived hinge), Ig-derived hinges containing a CD2 domain and a CD3 domain, Ig-derived hinges containing a CD2 domain and lacking a CD3 domain, Ig-derived hinges containing a CD3 domain and lacking a CD2 domain, Ig-derived hinges lacking a CD2 domain and lacking a CD3 domain, CD8α-derived hinges, CD28-derived hinges, and CD3ζ-derived hinges. A CAR provided herein can be designed to include a hinge of any appropriate length. For example, a CAR provided herein can be designed to include a hinge that is from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. In some cases, a linker sequence can be used as hinge to make a CAR described herein. For example, any one of the linker sequences set forth in FIG. 11 can be used as a hinge of a CAR described herein.

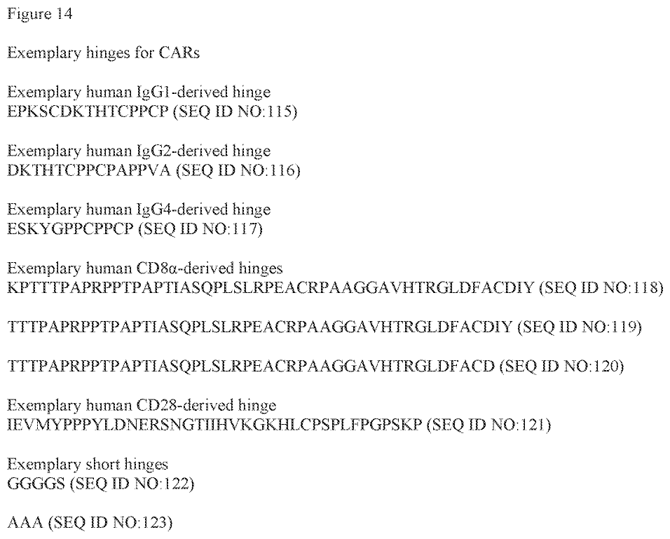

In some cases, a CAR provided herein can be designed to include a hinge that comprises, consists essentially of, or consists of one of the amino acid sequences set forth in FIG. 11 or FIG. 14. In some cases, a CAR provided herein can be designed to include a hinge that comprises, consists essentially of, or consists of one of the amino acid sequences set forth in FIG. 11 or FIG. 14 with one, two, three, four, five, six, seven, eight, nine, or ten amino acid deletions, additions, substitutions, or combinations thereof. In some cases, a CAR provided herein can be designed to include a hinge that comprises, consists essentially of, or consists of one of the amino acid sequences set forth in FIG. 11 or FIG. 14 with two or less, three or less, four or less, five or less, six or less, seven or less, eight or less, nine or less, or ten or less amino acid deletions, additions, substitutions, or combinations thereof.

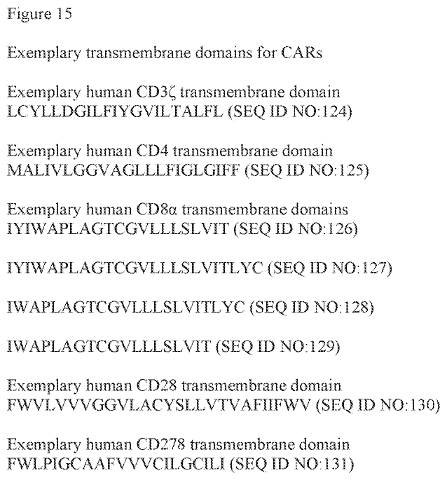

A CAR provided herein can be designed to include any appropriate transmembrane domain. For example, the transmembrane domain of a CAR provided herein can be, without limitation, a CD3ζ transmembrane domain, a CD4 transmembrane domain, a CD8a transmembrane domain, a CD28 transmembrane domain, and a 4-1BB transmembrane domain. In some cases, a CAR provided herein can be designed to include a transmembrane domain that comprises, consists essentially of, or consists of one of the amino acid sequences set forth in FIG. 15. In some cases, a CAR provided herein can be designed to include a transmembrane domain that comprises, consists essentially of, or consists of one of the amino acid sequences set forth in FIG. 15 with one, two, three, four, five, six, seven, eight, nine, or ten amino acid deletions, additions, substitutions, or combinations thereof. In some cases, a CAR provided herein can be designed to include a transmembrane domain that comprises, consists essentially of, or consists of one of the amino acid sequences set forth in FIG. 15 with two or less, three or less, four or less, five or less, six or less, seven or less, eight or less, nine or less, or ten or less amino acid deletions, additions, substitutions, or combinations thereof.

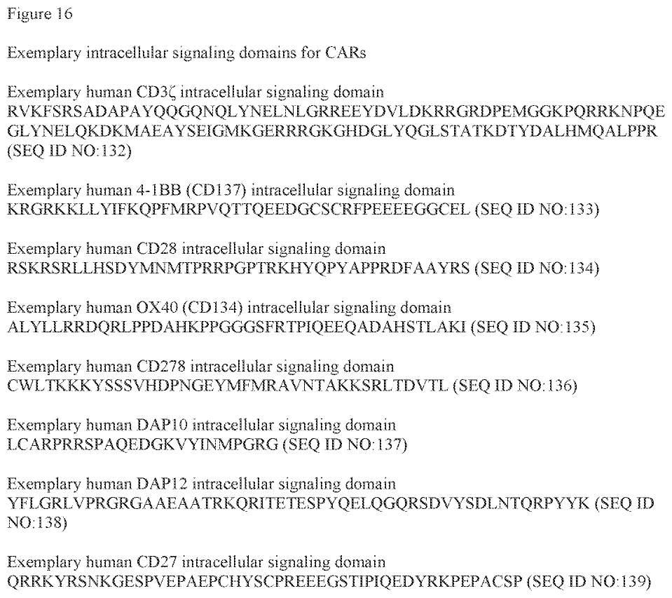

A CAR provided herein can be designed to include one or more intracellular signaling domains. For example, a CAR provided herein can be designed to include one, two, three, or four intracellular signaling domains. Any appropriate intracellular signaling domain or combination of intracellular signaling domains can be used to make a CAR described herein. Examples of intracellular signaling domains that can be used to make a CAR described herein include, without limitation, CD3ζ intracellular signaling domains, CD27 intracellular signaling domains, CD28 intracellular signaling domains, OX40 (CD134) intracellular signaling domains, 4-1BB (CD137) intracellular signaling domains, CD278 intracellular signaling domains, DAP10 intracellular signaling domains, and DAP12 intracellular signaling domains. In some cases, a CAR described herein can be designed to be a first generation CAR having a CD3ζ intracellular signaling domain. In some cases, a CAR described herein can be designed to be a second generation CAR having a CD28 intracellular signaling domain followed by a CD3ζ intracellular signaling domain. In some cases, a CAR described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ intracellular signaling domain. In some cases, a CAR provided herein can be designed to include at least one intracellular signaling domain that comprises, consists essentially of, or consists of one of the amino acid sequences set forth in FIG. 16. In some cases, a CAR provided herein can be designed to include at least one intracellular signaling domain that comprises, consists essentially of, or consists of one of the amino acid sequences set forth in FIG. 16 with one, two, three, four, five, six, seven, eight, nine, or ten amino acid deletions, additions, substitutions, or combinations thereof, provided that that intracellular signaling domain has at least some activity to activate intracellular signaling. In some cases, a CAR provided herein can be designed to include at least one intracellular signaling domain that comprises, consists essentially of, or consists of one of the amino acid sequences set forth in FIG. 16 with two or less, three or less, four or less, five or less, six or less, seven or less, eight or less, nine or less, or ten or less amino acid deletions, additions, substitutions, or combinations thereof, provided that that intracellular signaling domain has at least some activity to activate intracellular signaling.

In some cases, a CAR targeting an IL1RAP polypeptide can be designed to include a VH domain comprising SEQ ID NO:42, SEQ ID NO:43, and SEQ ID NO:44, followed by a hinge such as a hinge/linker set forth in FIG. 11 or FIG. 14 (e.g., a human CD8a hinge), followed by a transmembrane domain such as a transmembrane domain set forth in FIG. 15 (e.g., a human CD8a transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:42, SEQ ID NO:43, and SEQ ID NO:44, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO: 196, SEQ ID NO:197, and SEQ ID NO:198, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:196, SEQ ID NO:197, and SEQ ID NO:198, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:01, followed by a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:01, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:45, SEQ ID NO:46, and SEQ ID NO:47, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:45, SEQ ID NO:46, and SEQ ID NO:47, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:203, SEQ ID NO:204, and SEQ ID NO:205, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:203, SEQ ID NO:204, and SEQ ID NO:205, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:02, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:02, followed by SEQ ID NO:118, followed by SEQ ID NO: 126, followed by SEQ ID NO:133, followed by SEQ ID NO: 132.

In some cases, a CAR targeting an IL1RAP polypeptide can be designed to include a VH domain comprising SEQ ID NO:48, SEQ ID NO:49, and SEQ ID NO:50, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:48, SEQ ID NO:49, and SEQ ID NO:50, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:210, SEQ ID NO:211, and SEQ ID NO:212, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:210, SEQ ID NO:211, and SEQ ID NO:212, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:03, followed by a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:03, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:51, SEQ ID NO:52, and SEQ ID NO:53, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:51, SEQ ID NO:52, and SEQ ID NO:53, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:217, SEQ ID NO:218, and SEQ ID NO:219, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:217, SEQ ID NO:218, and SEQ ID NO:219, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:04, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:04, followed by SEQ ID NO:118, followed by SEQ ID NO: 126, followed by SEQ ID NO:133, followed by SEQ ID NO: 132.

In some cases, a CAR targeting an IL1RAP polypeptide can be designed to include a VH domain comprising SEQ ID NO:161, SEQ ID NO:162, and SEQ ID NO:163, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:161, SEQ ID NO:162, and SEQ ID NO:163, followed by SEQ ID NO: 118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:160, followed by a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:160, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO: 169, SEQ ID NO:170, and SEQ ID NO:171, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:169, SEQ ID NO:170, and SEQ ID NO:171, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:168, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:168, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO: 177, SEQ ID NO:178, and SEQ ID NO:179, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:177, SEQ ID NO:178, and SEQ ID NO:179, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:176, followed by a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:176, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:185, SEQ ID NO:186, and SEQ ID NO:187, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:185, SEQ ID NO:186, and SEQ ID NO:187, followed by SEQ ID NO:118, followed by SEQ ID NO:126, followed by SEQ ID NO:133, followed by SEQ ID NO:132.

In some cases, a CAR targeting an IL1RAP polypeptide can be designed to include a VH domain comprising SEQ ID NO:184, followed by a hinge such as a hinge/linker set forth in FIG. 11 or FIG. 14 (e.g., a human CD8α hinge), followed by a transmembrane domain such as a transmembrane domain set forth in FIG. 15 (e.g., a human CD8α transmembrane domain), followed by one or more intracellular signaling domains such as one or more intracellular signaling domain set forth in FIG. 16 (e.g., a human 4-1BB intracellular signaling domain followed by a human CD3ζ intracellular signaling domain). For example, a CAR targeting an IL1RAP polypeptide can be designed to include a VH domain comprising SEQ ID NO:184, followed by SEQ ID NO:118, followed by SEQ ID NO:126, followed by SEQ ID NO:133, followed by SEQ ID NO:132.

The term “cell engager” as used herein refers to a polypeptide that includes two or more antigen binding domains (e.g., two, three, or four antigen binding domains) and has the ability to link two cells together. Examples of cell engagers include, without limitation, BiTEs, BiKEs, and TriKEs. In general, a cell engager provided herein can be designed to include at least one antigen binding domain having the ability to bind to an IL1RAP polypeptide (e.g., a human IL1RAP polypeptide) and at least one antigen binding domain having the ability to bind to an antigen expressed on the surface of a cell (e.g., a T cell or an NK cell). In some cases, a cell engager described herein can link an IL1RAP⁺ cell (e.g., an IL1RAP⁺ cancer cell) to another cell (e.g., a T cell or an NK cell) via the two or more antigen binding domains of the cell engager.

When a cell engager includes an antigen binding domain having the ability to bind to an IL1RAP polypeptide (e.g., a human IL1RAP polypeptide) and two or more other antigen binding domains (e.g., two, three, or four other antigen binding domains), each of those other antigen binding domains can bind to different antigens expressed on the surface of different cell types or can bind to different antigens expressed on the surface of the same cell type. For example, a TriKE can be designed to have a first antigen binding domain having the ability to bind to an IL1RAP polypeptide (e.g., a human IL1RAP polypeptide), a second antigen binding domain having the ability to bind to a first antigen expressed on the surface of an NK cell (e.g., a CD16 polypeptide such as a CD16a polypeptide), and a third antigen binding domain having the ability to bind to a second antigen expressed on the surface of an NK cell (e.g., an NKG2A polypeptide).

As described herein, at least one antigen binding domain of a cell engager provided herein can be designed to bind to an IL1RAP polypeptide (e.g., a human IL1RAP polypeptide). For example, a cell engager provided herein can be designed to include the components of an antibody, antigen binding fragment, and/or antibody domain described herein (e.g., a combination of CDRs) as an antigen binding domain provided that that antigen binding domain has the ability to bind to an IL1RAP polypeptide (e.g., a human IL1RAP polypeptide). In some examples, a cell engager provided herein can be designed to include an antigen binding domain that includes a single set of three CDRs (e.g., a CDR1, CDR2, and CDR3) of an antibody domain (e.g., a VH domain) provided herein (e.g., SEQ ID NOs:42-44; SEQ ID NOs:196-198; SEQ ID NOs:45-47; SEQ ID NOs:203-205; SEQ ID NOs:48-50; SEQ ID NOs:210-212; SEQ ID NOs:51-53; SEQ ID NOs:217-219; SEQ ID NOs:161-163; SEQ ID NOs:169-171; SEQ ID NOs:177-179; or SEQ ID NOs:185-187). In some cases, an antigen binding domain of a cell engager targeting an IL1RAP polypeptide can be designed to include a VH domain described herein. In some cases, an antigen binding domain of a CAR described herein that has the ability to bind to an IL1RAP polypeptide (e.g., a human IL1RAP polypeptide) can be used as an antigen binding domain of a cell engager that targets IL1RAP⁺ cells.

As described herein, a cell engager can be designed to include at least one antigen binding domain having the ability to bind to an IL1RAP polypeptide (e.g., a human IL1RAP polypeptide) and at least one other antigen binding domain. That at least one other antigen binding domain can have the ability to bind to any appropriate antigen expressed on the surface of a cell. For example, when designing a cell engager such as a BiTE to link an IL1RAP⁺ cell and a T cell, the cell engager can include an antigen binding domain having the ability to bind to an IL1RAP polypeptide (e.g., a human IL1RAP polypeptide) and an antigen binding domain having the ability to bind to a polypeptide expressed on the surface of a T cell. Examples of polypeptides expressed on the surface of a T cell that can be targeted by an antigen binding domain of a cell engager provided herein include, without limitation, CD3 polypeptides. Examples of antigen binding domains having the ability to bind to a polypeptide expressed on the surface of a T cell that can be used to make a cell engager provided herein (e.g., a BiTE) include, without limitation, anti-CD3 scFvs and anti-CD3 VH domains. Additional examples of amino acid sequences that can be used as antigen binding domains having the ability to bind to a polypeptide expressed on the surface of a T cell (e.g., CD3) are described in U.S. Pat. No. 6,750,325 (see, e.g., the sequence listing of U.S. Pat. No. 6,750,325).

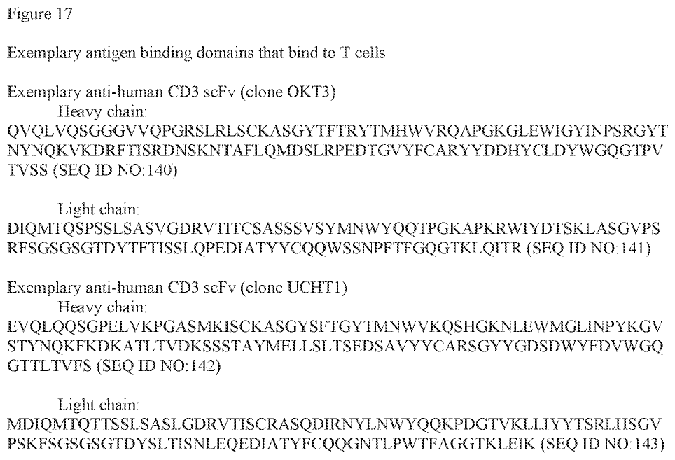

In some cases, a cell engager provided herein can be designed to include an antigen binding domain that comprises, consists essentially of, or consists of one of the amino acid sequences set forth in FIG. 17. In some cases, a cell engager provided herein can be designed to include an antigen binding domain that comprises, consists essentially of, or consists of one of the amino acid sequences set forth in FIG. 17 with one, two, three, four, five, six, seven, eight, nine, or ten amino acid deletions, additions, substitutions, or combinations thereof, provided that the antigen binding domain has the ability to bind to a polypeptide expressed on the surface of a T cell. In some cases, a cell engager provided herein can be designed to include an antigen binding domain that comprises, consists essentially of, or consists of one of the amino acid sequences set forth in FIG. 17 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of a T cell.

When designing a cell engager such as a BiKE or a TriKE to link an IL1RAP⁺ cell and an NK cell, the cell engager can include an antigen binding domain having the ability to bind to an IL1RAP polypeptide (e.g., a human IL1RAP polypeptide) and one or more (e.g., one, two, or three) antigen binding domains having the ability to bind to a polypeptide expressed on the surface of an NK cell. Examples of polypeptides expressed on the surface of an NK cell that can be targeted by an antigen binding domain of a cell engager provided herein include, without limitation, CD16 polypeptides (e.g., CD16a polypeptides), NKG2A polypeptides, NKG2D polypeptides, NKp30 polypeptides, NKp44 polypeptides, and NKp46 polypeptides. Examples of antigen binding domains having the ability to bind to a polypeptide expressed on the surface of an NK cell that can be used to make a cell engager provided herein (e.g., a BiKE or TriKE) include, without limitation, anti-CD16a scFvs, anti-NKG2A scFvs, anti-NKG2D scFvs, anti-NKp30 scFvs (see, e.g., BioLegend Catalog #325207), anti-NKp44 scFvs, anti-NKp46 scFvs, anti-CD16a VH domains, anti-NKG2A VH domains, anti-NKG2D VH domains, anti-NKp30 VH domains, anti-NKp44 VH domains, and anti-NKp46 VH domains. Additional examples of amino acid sequences that can be used as antigen binding domains having the ability to bind to a polypeptide expressed on the surface of an NK cell (e.g., CD16, NKG2A, NKG2D, or NKp46) are described in McCall et al. (Mol. Immunol., 36(7):433-445 (1999); see, e.g., anti-CD16 scFv sequences); International Patent Application Publication No. PCT/US2017/048721 (see, e.g., the CDRs and sequence listing for anti-CD16a binding domains); U.S. Patent Application Publication No. 2011/0052606 (see, e.g., the CDRs and the sequence listing for anti-NKG2A antibodies such as Z199); U.S. Patent Application Publication No. 2011/0150870 (see, e.g., the CDRs and sequence listing for anti-NKG2D antibodies); U.S. Patent Application Publication No. 2018/0369373 (see, e.g., the CDRs and sequence listing for anti-NKp46 antibodies); and U.S. Patent Application Publication No. 2017/0368169 (see, e.g., the CDRs and sequence listing for anti-NKp46 antibodies).

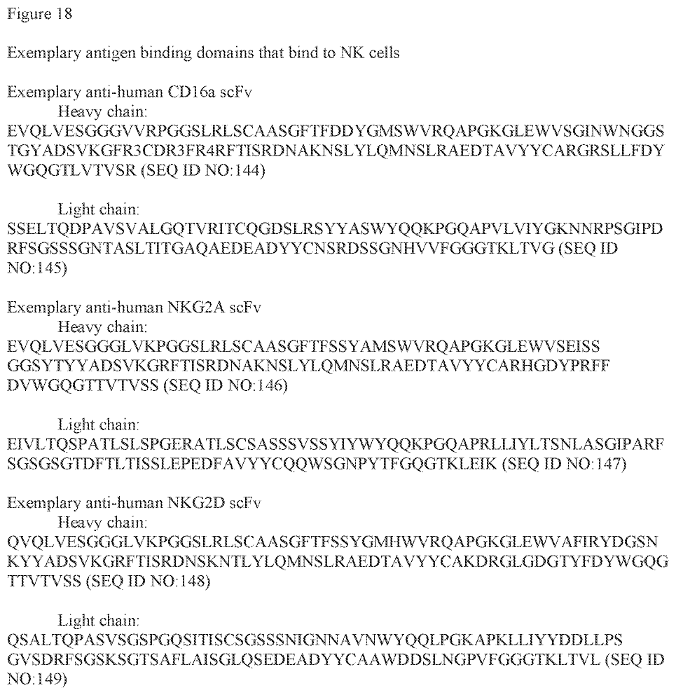

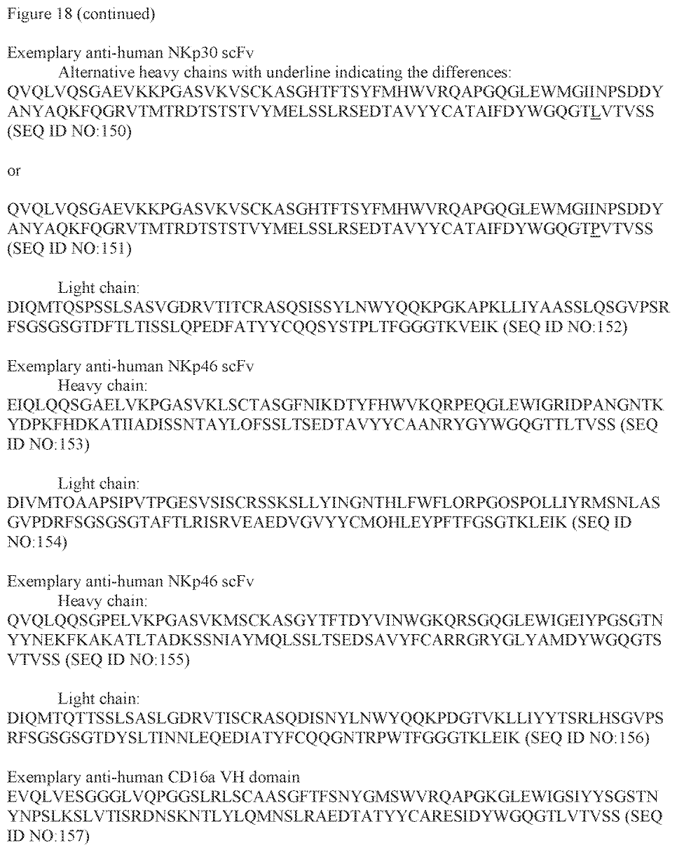

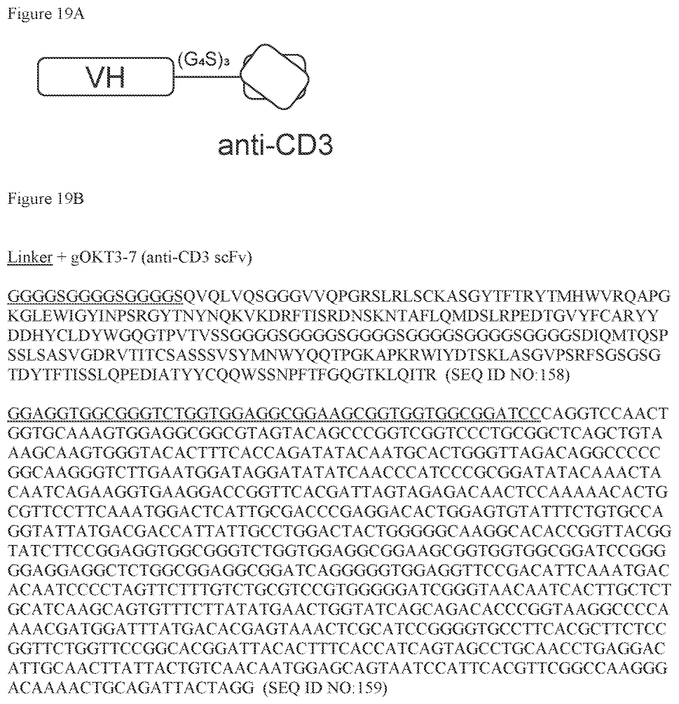

In some cases, a cell engager provided herein can be designed to include an antigen binding domain (e.g., a scFv or VH) that comprises, consists essentially of, or consists of one or more of the amino acid sequences set forth in FIG. 18. In some cases, a cell engager provided herein can be designed to include an antigen binding domain (e.g., a scFv or VH) that comprises, consists essentially of, or consists of one of the amino acid sequences set forth in FIG. 18 with one, two, three, four, five, six, seven, eight, nine, or ten amino acid deletions, additions, substitutions, or combinations thereof, provided that the antigen binding domain has the ability to bind to a polypeptide expressed on the surface of an NK cell. In some cases, a cell engager provided herein can be designed to include an antigen binding domain (e.g., a scFv or VH) that comprises, consists essentially of, or consists of one of the amino acid sequences set forth in FIG. 18 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of an NK cell.

In some cases, a cell engager provided herein can be designed to include a linker located between each antigen binding domain. Any appropriate linker can be used to design a cell engager provided herein. Examples of linkers that can be used to make a cell engager described herein include, without limitation, the linker sequences set forth in FIG. 11. A cell engager provided herein can be designed to include a linker of any appropriate length. For example, a cell engager provided herein can be designed to include a linker that is from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. In some cases, a cell engager provided herein (e.g., a BiTE) can be designed to include a GGGGSGGGGSGGGGS (SEQ ID NO:74) linker. In some cases, a hinge of a CAR described herein can be used as a linker to make a cell engager described herein. For example, any one of the sequences set forth in FIG. 14 can be used as a linker of a cell engager described herein.

In some cases, a cell engager provided herein can be designed to include a linker that comprises, consists essentially of, or consists of one of the amino acid sequences set forth in FIG. 11 or FIG. 14. In some cases, a cell engager provided herein can be designed to include a linker that comprises, consists essentially of, or consists of one of the amino acid sequences set forth in FIG. 11 or FIG. 14 with one, two, three, four, five, six, seven, eight, nine, or ten amino acid deletions, additions, substitutions, or combinations thereof. In some cases, a cell engager provided herein can be designed to include a linker that comprises, consists essentially of, or consists of one of the amino acid sequences set forth in FIG. 11 or FIG. 14 with two or less, three or less, four or less, five or less, six or less, seven or less, eight or less, nine or less, or ten or less amino acid deletions, additions, substitutions, or combinations thereof.

In some cases, a cell engager (e.g., a BiTE) targeting an IL1RAP polypeptide can be designed to include a VH domain comprising SEQ ID NO:42, SEQ ID NO:43, and SEQ ID NO:44, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiTE) targeting an IL1RAP polypeptide can be designed to include a VH domain comprising SEQ ID NO: 196, SEQ ID NO:197, and SEQ ID NO:198, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiTE) targeting an IL1RAP polypeptide can be designed to include a VH domain comprising SEQ ID NO:01, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiKE or a TriKE) targeting an IL1RAP polypeptide can be designed to include a VH domain comprising SEQ ID NO:42, SEQ ID NO:43, and SEQ ID NO:44, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an IL1RAP polypeptide can be designed to include a VH domain comprising SEQ ID NO:196, SEQ ID NO:197, and SEQ ID NO:198, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an IL1RAP polypeptide can be designed to include a VH domain comprising SEQ ID NO:01, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting an IL1RAP polypeptide can be designed to include a VH domain comprising SEQ ID NO:45, SEQ ID NO:46, and SEQ ID NO:47, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiTE) targeting an IL1RAP polypeptide can be designed to include a VH domain comprising SEQ ID NO:203, SEQ ID NO:204, and SEQ ID NO:205, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiTE) targeting an IL1RAP polypeptide can be designed to include a VH domain comprising SEQ ID NO:02, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiKE or a TriKE) targeting an IL1RAP polypeptide can be designed to include a VH domain comprising SEQ ID NO:45, SEQ ID NO:46, and SEQ ID NO:47, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an IL1RAP polypeptide can be designed to include a VH domain comprising SEQ ID NO:203, SEQ ID NO:204, and SEQ ID NO:205, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an IL1RAP polypeptide can be designed to include a VH domain comprising SEQ ID NO:02, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting an IL1RAP polypeptide can be designed to include a VH domain comprising SEQ ID NO:48, SEQ ID NO:49, and SEQ ID NO:50, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiTE) targeting an IL1RAP polypeptide can be designed to include a VH domain comprising SEQ ID NO:210, SEQ ID NO:211, and SEQ ID NO:212, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiTE) targeting an IL1RAP polypeptide can be designed to include a VH domain comprising SEQ ID NO:03, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiKE or a TriKE) targeting an IL1RAP polypeptide can be designed to include a VH domain comprising SEQ ID NO:48, SEQ ID NO:49, and SEQ ID NO:50, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an IL1RAP polypeptide can be designed to include a VH domain comprising SEQ ID NO:210, SEQ ID NO:211, and SEQ ID NO:212, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an IL1RAP polypeptide can be designed to include a VH domain comprising SEQ ID NO:03, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting an IL1RAP polypeptide can be designed to include a VH domain comprising SEQ ID NO:51, SEQ ID NO:52, and SEQ ID NO:53, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiTE) targeting an IL1RAP polypeptide can be designed to include a VH domain comprising SEQ ID NO:217, SEQ ID NO:218, and SEQ ID NO:219, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiTE) targeting an IL1RAP polypeptide can be designed to include a VH domain comprising SEQ ID NO:04, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiKE or a TriKE) targeting an IL1RAP polypeptide can be designed to include a VH domain comprising SEQ ID NO:51, SEQ ID NO:52, and SEQ ID NO:53, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an IL1RAP polypeptide can be designed to include a VH domain comprising SEQ ID NO:217, SEQ ID NO:218, and SEQ ID NO:219, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an IL1RAP polypeptide can be designed to include a VH domain comprising SEQ ID NO:04, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting an IL1RAP polypeptide can be designed to include a VH domain comprising SEQ ID NO:161, SEQ ID NO:162, and SEQ ID NO:163, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiTE) targeting an IL1RAP polypeptide can be designed to include a VH domain comprising SEQ ID NO:160, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiKE or a TriKE) targeting an IL1RAP polypeptide can be designed to include a VH domain comprising SEQ ID NO:161, SEQ ID NO:162, and SEQ ID NO:163, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an IL1RAP polypeptide can be designed to include a VH domain comprising SEQ ID NO:160, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting an IL1RAP polypeptide can be designed to include a VH domain comprising SEQ ID NO: 169, SEQ ID NO:170, and SEQ ID NO:171, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiTE) targeting an IL1RAP polypeptide can be designed to include a VH domain comprising SEQ ID NO:168, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiKE or a TriKE) targeting an IL1RAP polypeptide can be designed to include a VH domain comprising SEQ ID NO:169, SEQ ID NO:170, and SEQ ID NO:171, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an IL1RAP polypeptide can be designed to include a VH domain comprising SEQ ID NO:168, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting an IL1RAP polypeptide can be designed to include a VH domain comprising SEQ ID NO: 177, SEQ ID NO:178, and SEQ ID NO:179, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiTE) targeting an IL1RAP polypeptide can be designed to include a VH domain comprising SEQ ID NO:176, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiKE or a TriKE) targeting an IL1RAP polypeptide can be designed to include a VH domain comprising SEQ ID NO:177, SEQ ID NO:178, and SEQ ID NO:179, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an IL1RAP polypeptide can be designed to include a VH domain comprising SEQ ID NO:176, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting an IL1RAP polypeptide can be designed to include a VH domain comprising SEQ ID NO: 185, SEQ ID NO:186, and SEQ ID NO:187, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiTE) targeting an IL1RAP polypeptide can be designed to include a VH domain comprising SEQ ID NO:184, followed by a linker such as a hinge/linker set forth in FIG. 11 or FIG. 14 (e.g., SEQ ID NO:74), followed by an antigen binding domain having the ability to bind to a polypeptide expressed on the surface of a T cell (e.g., an anti-human CD3 scFv).

In some cases, a cell engager (e.g., a BiKE or a TriKE) targeting an IL1RAP polypeptide can be designed to include a VH domain comprising SEQ ID NO:185, SEQ ID NO:186, and SEQ ID NO:187, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting an IL1RAP polypeptide can be designed to include a VH domain comprising SEQ ID NO:184, followed by a linker such as a hinge/linker set forth in FIG. 11 or FIG. 14 (e.g., SEQ ID NO:74), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

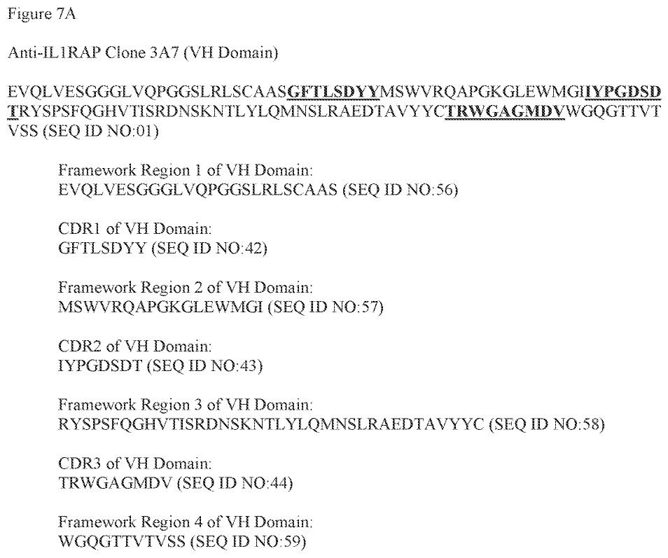

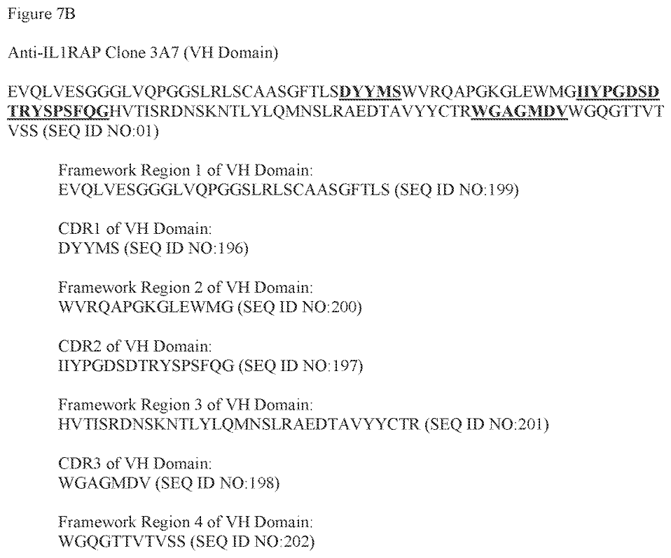

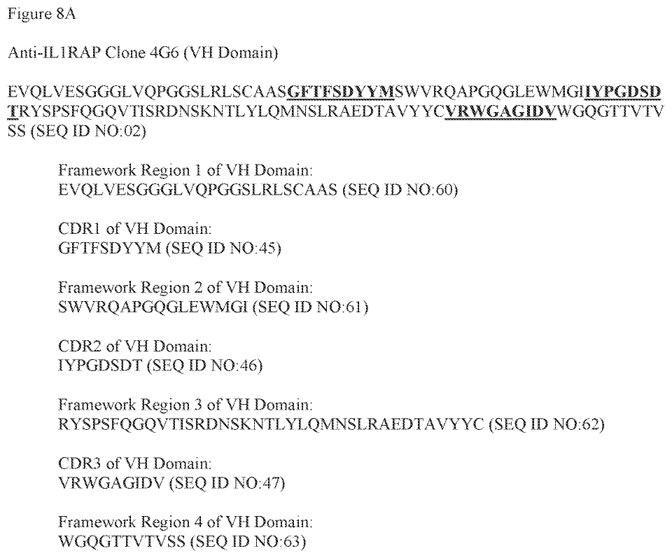

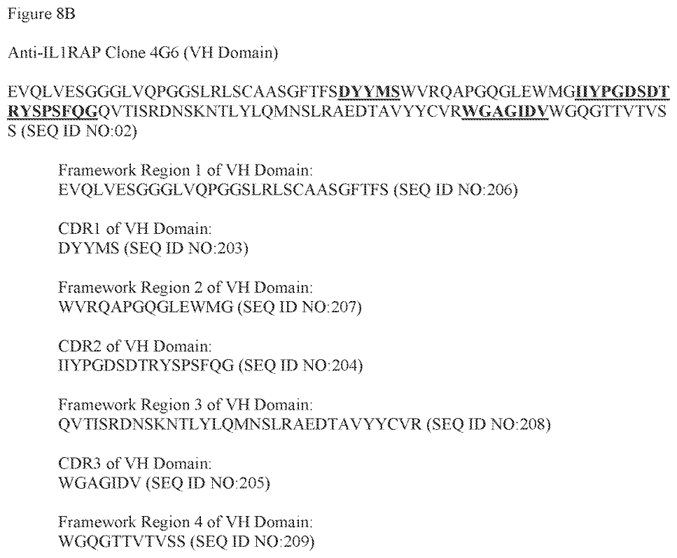

In one embodiment, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having a CDR1 having the amino acid sequence set forth in SEQ ID NO:42 (or a variant of SEQ ID NO:42 with one or two amino acid modifications), a CDR2 having the amino acid sequence set forth in SEQ ID NO:43 (or a variant of SEQ ID NO:43 with one or two amino acid modifications), and a CDR3 having the amino acid sequence set forth in SEQ ID NO:44 (or a variant of SEQ ID NO:44 with one or two amino acid modifications). An example of such an antibody domain having these CDRs and the ability to bind to an IL1RAP polypeptide (e.g., a human IL1RAP polypeptide) includes, without limitation, the VH domain set forth in FIG. 7A.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) and having a heavy chain variable domain having a CDR1 having the amino acid sequence set forth in SEQ ID NO:42 (or a variant of SEQ ID NO:42 with one or two amino acid modifications), a CDR2 having the amino acid sequence set forth in SEQ ID NO:43 (or a variant of SEQ ID NO:43 with one or two amino acid modifications), and a CDR3 having the amino acid sequence set forth in SEQ ID NO:44 (or a variant of SEQ ID NO:44 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:56 (or a variant of SEQ ID NO:56 with one, two, three, four, five, six, seven, eight, nine, ten, or more amino acid modifications), a framework region 2 having the amino acid sequence set forth in SEQ ID NO:57 (or a variant of SEQ ID NO:57 with one, two, three, four, five, six, seven, eight, nine, ten, or more amino acid modifications), a framework region 3 having the amino acid sequence set forth in SEQ ID NO:58 (or a variant of SEQ ID NO:58 with one, two, three, four, five, six, seven, eight, nine, ten, or more amino acid modifications), and a framework region 4 having the amino acid sequence set forth in SEQ ID NO:59 (or a variant of SEQ ID NO:59 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 7A can be designed to include framework regions as set forth in FIG. 7A or can be designed to include one or more framework regions from another antibody or antibody fragment. For example, an antibody domain (e.g., a VH domain) can be designed to include the three CDRs set forth in FIG. 7A and the framework regions set forth in FIG. 7A except that framework region 1 having the amino acid set forth in SEQ ID NO:56 is replaced with a framework region 1 having the amino acid set forth in SEQ ID NO:60, a framework region 1 having the amino acid set forth in SEQ ID NO:64, or a framework region 1 having the amino acid set forth in SEQ ID NO:68.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:01. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:01. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:01.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:01, provided that the heavy chain variable domain includes the amino acid sequences set forth in SEQ ID NOs:42, 43, and 44. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:01, provided that the heavy chain variable domain includes the amino acid sequences set forth in SEQ ID NOs:42, 43, and 44.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having the amino acid sequence set forth in SEQ ID NO:01 or the amino acid set forth in SEQ ID NO:01 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to an IL1RAP polypeptide (e.g., a human IL1RAP polypeptide) and can include a heavy chain variable domain having the amino acid sequence set forth in SEQ ID NO:01 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:42, 43, and 44.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain comprising (i) a CDR1 that comprises, consists essentially of, or consists of the amino acid sequence set forth in SEQ ID NO:42, (ii) a CDR2 that comprises, consists essentially of, or consists of the amino acid sequence set forth in SEQ ID NO:43, and (iii) a CDR3 that comprises, consists essentially of, or consists of the amino acid sequence set forth in SEQ ID NO:44.

As used herein, a “CDR1 that consists essentially of the amino acid sequence set forth in SEQ ID NO:42” is a CDR1 that has zero, one, or two amino acid substitutions within SEQ ID NO:42, that has zero, one, two, three, four, or five amino acid residues directly preceding SEQ ID NO:42, and/or that has zero, one, two, three, four, or five amino acid residues directly following SEQ ID NO:42, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide).

As used herein, a “CDR2 that consists essentially of the amino acid sequence set forth in SEQ ID NO:43” is a CDR2 that has zero, one, or two amino acid substitutions within SEQ ID NO:43, that has zero, one, two, three, four, or five amino acid residues directly preceding SEQ ID NO:43, and/or that has zero, one, two, three, four, or five amino acid residues directly following SEQ ID NO:43, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide).

As used herein, a “CDR3 that consists essentially of the amino acid sequence set forth in SEQ ID NO:44” is a CDR3 that has zero, one, or two amino acid substitutions within SEQ ID NO:44, that has zero, one, two, three, four, or five amino acid residues directly preceding SEQ ID NO:44, and/or that has zero, one, two, three, four, or five amino acid residues directly following SEQ ID NO:44, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide).

In another embodiment, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having a CDR1 having the amino acid sequence set forth in SEQ ID NO:196 (or a variant of SEQ ID NO:196 with one or two amino acid modifications), a CDR2 having the amino acid sequence set forth in SEQ ID NO: 197 (or a variant of SEQ ID NO:197 with one or two amino acid modifications), and a CDR3 having the amino acid sequence set forth in SEQ ID NO:198 (or a variant of SEQ ID NO:198 with one or two amino acid modifications). An example of such an antibody domain having these CDRs and the ability to bind to an IL1RAP polypeptide (e.g., a human IL1RAP polypeptide) includes, without limitation, the VH domain set forth in FIG. 7B.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) and having a heavy chain variable domain having a CDR1 having the amino acid sequence set forth in SEQ ID NO:196 (or a variant of SEQ ID NO:196 with one or two amino acid modifications), a CDR2 having the amino acid sequence set forth in SEQ ID NO: 197 (or a variant of SEQ ID NO: 197 with one or two amino acid modifications), and a CDR3 having the amino acid sequence set forth in SEQ ID NO:198 (or a variant of SEQ ID NO:198 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:199 (or a variant of SEQ ID NO:199 with one, two, three, four, five, six, seven, eight, nine, ten, or more amino acid modifications), a framework region 2 having the amino acid sequence set forth in SEQ ID NO:200 (or a variant of SEQ ID NO:200 with one, two, three, four, five, six, seven, eight, nine, ten, or more amino acid modifications), a framework region 3 having the amino acid sequence set forth in SEQ ID NO:201 (or a variant of SEQ ID NO:201 with one, two, three, four, five, six, seven, eight, nine, ten, or more amino acid modifications), and a framework region 4 having the amino acid sequence set forth in SEQ ID NO:202 (or a variant of SEQ ID NO:202 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 7B can be designed to include framework regions as set forth in FIG. 7B or can be designed to include one or more framework regions from another antibody or antibody fragment. For example, an antibody domain (e.g., a VH domain) can be designed to include the three CDRs set forth in FIG. 7B and the framework regions set forth in FIG. 7B except that framework region 1 having the amino acid set forth in SEQ ID NO:199 is replaced with a framework region 1 having the amino acid set forth in SEQ ID NO:206, a framework region 1 having the amino acid set forth in SEQ ID NO:213, or a framework region 1 having the amino acid set forth in SEQ ID NO:220.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:01, provided that the heavy chain variable domain includes the amino acid sequences set forth in SEQ ID NOs:196, 197, and 198. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:01, provided that the heavy chain variable domain includes the amino acid sequences set forth in SEQ ID NOs:196, 197, and 198.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having the amino acid sequence set forth in SEQ ID NO:01 or the amino acid set forth in SEQ ID NO:01 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to an IL1RAP polypeptide (e.g., a human IL1RAP polypeptide) and can include a heavy chain variable domain having the amino acid sequence set forth in SEQ ID NO:01 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:196, 197, and 198.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain comprising (i) a CDR1 that comprises, consists essentially of, or consists of the amino acid sequence set forth in SEQ ID NO: 196, (ii) a CDR2 that comprises, consists essentially of, or consists of the amino acid sequence set forth in SEQ ID NO:197, and (iii) a CDR3 that comprises, consists essentially of, or consists of the amino acid sequence set forth in SEQ ID NO:198. As used herein, a “CDR1 that consists essentially of the amino acid sequence set forth in SEQ ID NO:196” is a CDR1 that has zero, one, or two amino acid substitutions within SEQ ID NO:196, that has zero, one, two, three, four, or five amino acid residues directly preceding SEQ ID NO:196, and/or that has zero, one, two, three, four, or five amino acid residues directly following SEQ ID NO:196, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR1 that consists essentially of the amino acid sequence set forth in SEQ ID NO:196 include, without limitation, those set forth in Table 3.

TABLE 3
Exemplary CDR1s that consist essentially of the
amino acid sequence set forth in SEQ ID NO: 196.

| Sequence | SEQ ID NO: |
|---|---|
| NYYMS | 224 |
| NNYMS | 225 |
| NNYMH | 226 |
| NNYMG | 227 |
| NNYME | 228 |
| DYYMH | 229 |
| DYYMD | 230 |
| DYYMG | 231 |
| NYYMH | 232 |
| EYYMH | 233 |

As used herein, a “CDR2 that consists essentially of the amino acid sequence set forth in SEQ ID NO:197” is a CDR2 that has zero, one, or two amino acid substitutions within SEQ ID NO: 197, that has zero, one, two, three, four, or five amino acid residues directly preceding SEQ TD NO: 197, and/or that has zero, one, two, three, four, or five amino acid residues directly following SEQ ID NO: 197, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR2 that consists essentially of the amino acid sequence set forth in SEQ ID NO: 197 include, without limitation, those set forth in Table 4.

TABLE 4
Exemplary CDR2s that consist essentially of the
amino acid sequence set forth in SEQ ID NO: 197.

| Sequence | SEQ ID NO: |
|---|---|
| IIYPGDSDTRYSPSFQS | 234 |
| IIYPGDSDTRYSPSFQA | 235 |
| IIYPGDSETRYSPSFQS | 236 |
| IIYPGDSETRYSPSFQG | 237 |
| IIYPGDTETRYSPSFQG | 238 |
| IIYPGDTDTRYSPSFQS | 239 |
| IIYPGDTDTRYTPSFQS | 240 |
| IIYPGDTDTRYTPSFQG | 241 |
| IIYPGDTDTRYTPSFNG | 242 |
| IIYPGETDTRYTPSFNG | 243 |

As used herein, a “CDR3 that consists essentially of the amino acid sequence set forth in SEQ ID NO: 198” is a CDR3 that has zero, one, or two amino acid substitutions within SEQ ID NO: 198, that has zero, one, two, three, four, or five amino acid residues directly preceding SEQ TD NO: 198, and/or that has zero, one, two, three, four, or five amino acid residues directly following SEQ ID NO: 198, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR3 that consists essentially of the amino acid sequence set forth in SEQ ID NO:198 include, without limitation, those set forth in Table 5.

TABLE 5
Exemplary CDR3s that consist essentially of the
amino acid sequence set forth in SEQ ID NO: 198.

| Sequence | SEQ ID NO: |
|---|---|
| WSAGMDV | 244 |
| WGASMDV | 245 |
| WGAGMDL | 246 |
| WGTGMDV | 247 |
| WGAGMEV | 248 |
| WGAGMEL | 249 |
| WTAGMEV | 250 |
| WGATMDL | 251 |
| WGATMEV | 252 |
| WGAGMDA | 253 |

In another embodiment, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having a CDR1 having the amino acid sequence set forth in SEQ ID NO:45 (or a variant of SEQ ID NO:45 with one or two amino acid modifications), a CDR2 having the amino acid sequence set forth in SEQ ID NO:46 (or a variant of SEQ ID NO:46 with one or two amino acid modifications), and a CDR3 having the amino acid sequence set forth in SEQ ID NO:47 (or a variant of SEQ ID NO:47 with one or two amino acid modifications). An example of such an antibody domain having these CDRs and the ability to bind to an IL1RAP polypeptide (e.g., a human IL1RAP polypeptide) includes, without limitation, the VH domain set forth in FIG. 8A.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) and having a heavy chain variable domain having a CDR1 having the amino acid sequence set forth in SEQ ID NO:45 (or a variant of SEQ ID NO:45 with one or two amino acid modifications), a CDR2 having the amino acid sequence set forth in SEQ ID NO:46 (or a variant of SEQ ID NO:46 with one or two amino acid modifications), and a CDR3 having the amino acid sequence set forth in SEQ ID NO:47 (or a variant of SEQ ID NO:47 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:60 (or a variant of SEQ ID NO:60 with one, two, three, four, five, six, seven, eight, nine, ten, or more amino acid modifications), a framework region 2 having the amino acid sequence set forth in SEQ ID NO:61 (or a variant of SEQ ID NO:61 with one, two, three, four, five, six, seven, eight, nine, ten, or more amino acid modifications), a framework region 3 having the amino acid sequence set forth in SEQ ID NO:62 (or a variant of SEQ ID NO:62 with one, two, three, four, five, six, seven, eight, nine, ten, or more amino acid modifications), and a framework region 4 having the amino acid sequence set forth in SEQ ID NO:63 (or a variant of SEQ ID NO:63 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 8A can be designed to include framework regions as set forth in FIG. 8A or can be designed to include one or more framework regions from another antibody or antibody fragment. For example, an antibody domain (e.g., a VH domain) can be designed to include the three CDRs set forth in FIG. 8A and the framework regions set forth in FIG. 8A except that framework region 1 having the amino acid set forth in SEQ ID NO:60 is replaced with a framework region 1 having the amino acid set forth in SEQ ID NO:56, a framework region 1 having the amino acid set forth in SEQ ID NO:64, or a framework region 1 having the amino acid set forth in SEQ ID NO:68.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:02. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:02. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:02.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:02, provided that the heavy chain variable domain includes the amino acid sequences set forth in SEQ ID NOs:45, 46, and 47. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:02, provided that the heavy chain variable domain includes the amino acid sequences set forth in SEQ ID NOs:45, 46, and 47.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having the amino acid sequence set forth in SEQ ID NO:02 or the amino acid set forth in SEQ ID NO:02 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to an IL1RAP polypeptide (e.g., a human IL1RAP polypeptide) and can include a heavy chain variable domain having the amino acid sequence set forth in SEQ ID NO:02 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:45, 46, and 47.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain comprising (i) a CDR1 that comprises, consists essentially of, or consists of the amino acid sequence set forth in SEQ ID NO:45, (ii) a CDR2 that comprises, consists essentially of, or consists of the amino acid sequence set forth in SEQ ID NO:46, and (iii) a CDR3 that comprises, consists essentially of, or consists of the amino acid sequence set forth in SEQ ID NO:47. As used herein, a “CDR1 that consists essentially of the amino acid sequence set forth in SEQ ID NO:45” is a CDR1 that has zero, one, or two amino acid substitutions within SEQ ID NO:45, that has zero, one, two, three, four, or five amino acid residues directly preceding SEQ ID NO:45, and/or that has zero, one, two, three, four, or five amino acid residues directly following SEQ ID NO:45, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide).

As used herein, a “CDR2 that consists essentially of the amino acid sequence set forth in SEQ ID NO:46” is a CDR2 that has zero, one, or two amino acid substitutions within SEQ ID NO:46, that has zero, one, two, three, four, or five amino acid residues directly preceding SEQ ID NO:46, and/or that has zero, one, two, three, four, or five amino acid residues directly following SEQ ID NO:46, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide).

As used herein, a “CDR3 that consists essentially of the amino acid sequence set forth in SEQ ID NO:47” is a CDR3 that has zero, one, or two amino acid substitutions within SEQ ID NO:47, that has zero, one, two, three, four, or five amino acid residues directly preceding SEQ ID NO:47, and/or that has zero, one, two, three, four, or five amino acid residues directly following SEQ ID NO:47, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide).

In another embodiment, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having a CDR1 having the amino acid sequence set forth in SEQ ID NO:203 (or a variant of SEQ ID NO:203 with one or two amino acid modifications), a CDR2 having the amino acid sequence set forth in SEQ ID NO:204 (or a variant of SEQ ID NO:204 with one or two amino acid modifications), and a CDR3 having the amino acid sequence set forth in SEQ ID NO:205 (or a variant of SEQ ID NO:205 with one or two amino acid modifications). An example of such an antibody domain having these CDRs and the ability to bind to an IL1RAP polypeptide (e.g., a human IL1RAP polypeptide) includes, without limitation, the VH domain set forth in FIG. 8B.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) and having a heavy chain variable domain having a CDR1 having the amino acid sequence set forth in SEQ ID NO:203 (or a variant of SEQ ID NO:203 with one or two amino acid modifications), a CDR2 having the amino acid sequence set forth in SEQ ID NO:204 (or a variant of SEQ ID NO:204 with one or two amino acid modifications), and a CDR3 having the amino acid sequence set forth in SEQ ID NO:205 (or a variant of SEQ ID NO:205 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:206 (or a variant of SEQ ID NO:206 with one, two, three, four, five, six, seven, eight, nine, ten, or more amino acid modifications), a framework region 2 having the amino acid sequence set forth in SEQ ID NO:207 (or a variant of SEQ ID NO:207 with one, two, three, four, five, six, seven, eight, nine, ten, or more amino acid modifications), a framework region 3 having the amino acid sequence set forth in SEQ ID NO:208 (or a variant of SEQ ID NO:208 with one, two, three, four, five, six, seven, eight, nine, ten, or more amino acid modifications), and a framework region 4 having the amino acid sequence set forth in SEQ ID NO:209 (or a variant of SEQ ID NO:209 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 8B can be designed to include framework regions as set forth in FIG. 8B or can be designed to include one or more framework regions from another antibody or antibody fragment. For example, an antibody domain (e.g., a VH domain) can be designed to include the three CDRs set forth in FIG. 8B and the framework regions set forth in FIG. 8B except that framework region 1 having the amino acid set forth in SEQ ID NO:206 is replaced with a framework region 1 having the amino acid set forth in SEQ ID NO:199, a framework region 1 having the amino acid set forth in SEQ ID NO:213, or a framework region 1 having the amino acid set forth in SEQ ID NO:220.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:02, provided that the heavy chain variable domain includes the amino acid sequences set forth in SEQ ID NOs:203, 204, and 205. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:02, provided that the heavy chain variable domain includes the amino acid sequences set forth in SEQ ID NOs:203, 204, and 205.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having the amino acid sequence set forth in SEQ ID NO:02 or the amino acid set forth in SEQ ID NO:02 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to an IL1RAP polypeptide (e.g., a human IL1RAP polypeptide) and can include a heavy chain variable domain having the amino acid sequence set forth in SEQ ID NO:02 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:203, 204, and 205.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain comprising (i) a CDR1 that comprises, consists essentially of, or consists of the amino acid sequence set forth in SEQ ID NO:203, (ii) a CDR2 that comprises, consists essentially of, or consists of the amino acid sequence set forth in SEQ ID NO:204, and (iii) a CDR3 that comprises, consists essentially of, or consists of the amino acid sequence set forth in SEQ ID NO:205. As used herein, a “CDR1 that consists essentially of the amino acid sequence set forth in SEQ ID NO:203” is a CDR1 that has zero, one, or two amino acid substitutions within SEQ ID NO:203, that has zero, one, two, three, four, or five amino acid residues directly preceding SEQ ID NO:203, and/or that has zero, one, two, three, four, or five amino acid residues directly following SEQ ID NO:203, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR1 that consists essentially of the amino acid sequence set forth in SEQ ID NO:203 include, without limitation, those set forth in Table 6.

TABLE 6
Exemplary CDR1s that consist essentially of the
amino acid sequence set forth in SEQ ID NO: 203.

| Sequence | SEQ ID NO: |
|---|---|
| NYYMS | 254 |
| NNYMS | 255 |
| NNYMH | 256 |
| NNYMG | 257 |
| NNYME | 258 |
| DYYMH | 259 |
| DYYMD | 260 |
| DYYMG | 261 |
| NYYMH | 262 |
| EYYMH | 263 |

As used herein, a “CDR2 that consists essentially of the amino acid sequence set forth in SEQ ID NO:204” is a CDR2 that has zero, one, or two amino acid substitutions within SEQ ID NO:204, that has zero, one, two, three, four, or five amino acid residues directly preceding SEQ ID NO:204, and/or that has zero, one, two, three, four, or five amino acid residues directly following SEQ ID NO:204, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR2 that consists essentially of the amino acid sequence set forth in SEQ ID NO:204 include, without limitation, those set forth in Table 7.

TABLE 7
Exemplary CDR2s that consist essentially of the
amino acid sequence set forth in SEQ ID NO: 204.

| Sequence | SEQ ID NO: |
|---|---|
| IIYPGDSDTRYSPSFQS | 264 |
| IIYPGDSDTRYSPSFQA | 265 |
| IIYPGDSETRYSPSFQS | 266 |
| IIYPGDSETRYSPSFQG | 267 |
| IIYPGDTETRYSPSFQG | 268 |
| IIYPGDTDTRYSPSFQS | 269 |
| IIYPGDTDTRYTPSFQS | 270 |
| IIYPGDTDTRYTPSFQG | 271 |
| IIYPGDTDTRYTPSFNG | 272 |
| IIYPGETDTRYTPSFNG | 273 |

As used herein, a “CDR3 that consists essentially of the amino acid sequence set forth in SEQ ID NO:205” is a CDR3 that has zero, one, or two amino acid substitutions within SEQ ID NO:205, that has zero, one, two, three, four, or five amino acid residues directly preceding SEQ ID NO:205, and/or that has zero, one, two, three, four, or five amino acid residues directly following SEQ ID NO:205, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR3 that consists essentially of the amino acid sequence set forth in SEQ ID NO:205 include, without limitation, those set forth in Table 8.

TABLE 8
Exemplary CDR3s that consist essentially of the
amino acid sequence set forth in SEQ ID NO: 205.

| Sequence | SEQ ID NO: |
|---|---|
| WSAGIDV | 274 |
| WGASIDV | 275 |
| WGAGIDL | 276 |
| WGTGIDV | 277 |
| WGAGIEV | 278 |
| WGAGIEL | 279 |
| WTAGIEV | 280 |
| WGATIDL | 281 |
| WGATIEV | 282 |
| WGAGIDA | 283 |

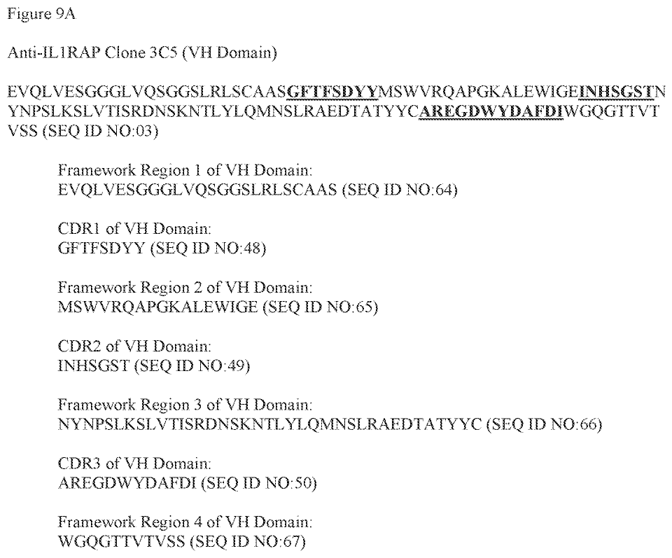

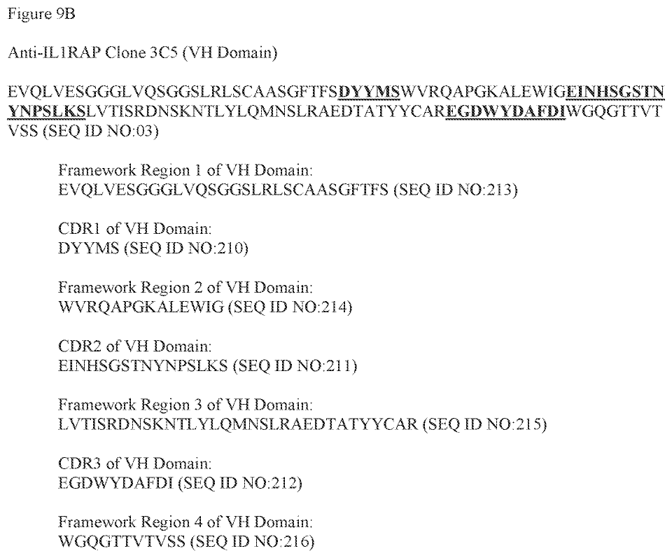

In another embodiment, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having a CDR1 having the amino acid sequence set forth in SEQ ID NO:48 (or a variant of SEQ ID NO:48 with one or two amino acid modifications), a CDR2 having the amino acid sequence set forth in SEQ ID NO:49 (or a variant of SEQ ID NO:49 with one or two amino acid modifications), and a CDR3 having the amino acid sequence set forth in SEQ ID NO:50 (or a variant of SEQ ID NO:50 with one or two amino acid modifications). An example of such an antibody domain having these CDRs and the ability to bind to an IL1RAP polypeptide (e.g., a human IL1RAP polypeptide) includes, without limitation, the VH domain set forth in FIG. 9A.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) and having a heavy chain variable domain having a CDR1 having the amino acid sequence set forth in SEQ ID NO:48 (or a variant of SEQ ID NO:48 with one or two amino acid modifications), a CDR2 having the amino acid sequence set forth in SEQ ID NO:49 (or a variant of SEQ ID NO:49 with one or two amino acid modifications), and a CDR3 having the amino acid sequence set forth in SEQ ID NO:50 (or a variant of SEQ ID NO:50 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:64 (or a variant of SEQ ID NO:64 with one, two, three, four, five, six, seven, eight, nine, ten, or more amino acid modifications), a framework region 2 having the amino acid sequence set forth in SEQ ID NO:65 (or a variant of SEQ ID NO:65 with one, two, three, four, five, six, seven, eight, nine, ten, or more amino acid modifications), a framework region 3 having the amino acid sequence set forth in SEQ ID NO:66 (or a variant of SEQ ID NO:66 with one, two, three, four, five, six, seven, eight, nine, ten, or more amino acid modifications), and a framework region 4 having the amino acid sequence set forth in SEQ ID NO:67 (or a variant of SEQ ID NO:67 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 9A can be designed to include framework regions as set forth in FIG. 9A or can be designed to include one or more framework regions from another antibody or antibody fragment. For example, an antibody domain (e.g., a VH domain) can be designed to include the three CDRs set forth in FIG. 9A and the framework regions set forth in FIG. 9A except that framework region 1 having the amino acid set forth in SEQ ID NO:64 is replaced with a framework region 1 having the amino acid set forth in SEQ ID NO:56, a framework region 1 having the amino acid set forth in SEQ ID NO:60, or a framework region 1 having the amino acid set forth in SEQ ID NO:68.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:03. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:03. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:03.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:03, provided that the heavy chain variable domain includes the amino acid sequences set forth in SEQ ID NOs:48, 49, and 50. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:03, provided that the heavy chain variable domain includes the amino acid sequences set forth in SEQ ID NOs:48, 49, and 50.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having the amino acid sequence set forth in SEQ ID NO:03 or the amino acid set forth in SEQ ID NO:03 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to an IL1RAP polypeptide (e.g., a human IL1RAP polypeptide) and can include a heavy chain variable domain having the amino acid sequence set forth in SEQ ID NO:03 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:48, 49, and 50.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain comprising (i) a CDR1 that comprises, consists essentially of, or consists of the amino acid sequence set forth in SEQ ID NO:48, (ii) a CDR2 that comprises, consists essentially of, or consists of the amino acid sequence set forth in SEQ ID NO:49, and (iii) a CDR3 that comprises, consists essentially of, or consists of the amino acid sequence set forth in SEQ ID NO:50.

As used herein, a “CDR1 that consists essentially of the amino acid sequence set forth in SEQ ID NO:48” is a CDR1 that has zero, one, or two amino acid substitutions within SEQ ID NO:48, that has zero, one, two, three, four, or five amino acid residues directly preceding SEQ ID NO:48, and/or that has zero, one, two, three, four, or five amino acid residues directly following SEQ ID NO:48, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide).

As used herein, a “CDR2 that consists essentially of the amino acid sequence set forth in SEQ ID NO:49” is a CDR2 that has zero, one, or two amino acid substitutions within SEQ ID NO:49, that has zero, one, two, three, four, or five amino acid residues directly preceding SEQ ID NO:49, and/or that has zero, one, two, three, four, or five amino acid residues directly following SEQ ID NO:49, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide).

As used herein, a “CDR3 that consists essentially of the amino acid sequence set forth in SEQ ID NO:50” is a CDR3 that has zero, one, or two amino acid substitutions within SEQ ID NO:50, that has zero, one, two, three, four, or five amino acid residues directly preceding SEQ ID NO:50, and/or that has zero, one, two, three, four, or five amino acid residues directly following SEQ ID NO:50, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide).

In another embodiment, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having a CDR1 having the amino acid sequence set forth in SEQ ID NO:210 (or a variant of SEQ ID NO:210 with one or two amino acid modifications), a CDR2 having the amino acid sequence set forth in SEQ ID NO:211 (or a variant of SEQ ID NO:211 with one or two amino acid modifications), and a CDR3 having the amino acid sequence set forth in SEQ ID NO:212 (or a variant of SEQ ID NO:212 with one or two amino acid modifications). An example of such an antibody domain having these CDRs and the ability to bind to an IL1RAP polypeptide (e.g., a human IL1RAP polypeptide) includes, without limitation, the VH domain set forth in FIG. 9B.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) and having a heavy chain variable domain having a CDR1 having the amino acid sequence set forth in SEQ ID NO:210 (or a variant of SEQ ID NO:210 with one or two amino acid modifications), a CDR2 having the amino acid sequence set forth in SEQ ID NO:211 (or a variant of SEQ ID NO:211 with one or two amino acid modifications), and a CDR3 having the amino acid sequence set forth in SEQ ID NO:212 (or a variant of SEQ ID NO:212 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:213 (or a variant of SEQ ID NO:213 with one, two, three, four, five, six, seven, eight, nine, ten, or more amino acid modifications), a framework region 2 having the amino acid sequence set forth in SEQ ID NO:214 (or a variant of SEQ ID NO:214 with one, two, three, four, five, six, seven, eight, nine, ten, or more amino acid modifications), a framework region 3 having the amino acid sequence set forth in SEQ ID NO:215 (or a variant of SEQ ID NO:215 with one, two, three, four, five, six, seven, eight, nine, ten, or more amino acid modifications), and a framework region 4 having the amino acid sequence set forth in SEQ ID NO:216 (or a variant of SEQ ID NO:216 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 9B can be designed to include framework regions as set forth in FIG. 9B or can be designed to include one or more framework regions from another antibody or antibody fragment. For example, an antibody domain (e.g., a VH domain) can be designed to include the three CDRs set forth in FIG. 9B and the framework regions set forth in FIG. 9B except that framework region 1 having the amino acid set forth in SEQ ID NO:213 is replaced with a framework region 1 having the amino acid set forth in SEQ ID NO:199, a framework region 1 having the amino acid set forth in SEQ ID NO:206, or a framework region 1 having the amino acid set forth in SEQ ID NO:220.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:03, provided that the heavy chain variable domain includes the amino acid sequences set forth in SEQ ID NOs:210, 211, and 212. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:03, provided that the heavy chain variable domain includes the amino acid sequences set forth in SEQ ID NOs:210, 211, and 212.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having the amino acid sequence set forth in SEQ ID NO:03 or the amino acid set forth in SEQ ID NO:03 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to an IL1RAP polypeptide (e.g., a human IL1RAP polypeptide) and can include a heavy chain variable domain having the amino acid sequence set forth in SEQ ID NO:03 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:210, 211, and 212.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain comprising (i) a CDR1 that comprises, consists essentially of, or consists of the amino acid sequence set forth in SEQ ID NO:210, (ii) a CDR2 that comprises, consists essentially of, or consists of the amino acid sequence set forth in SEQ ID NO:211, and (iii) a CDR3 that comprises, consists essentially of, or consists of the amino acid sequence set forth in SEQ ID NO:212. As used herein, a “CDR1 that consists essentially of the amino acid sequence set forth in SEQ ID NO:210” is a CDR1 that has zero, one, or two amino acid substitutions within SEQ ID NO:210, that has zero, one, two, three, four, or five amino acid residues directly preceding SEQ ID NO:210, and/or that has zero, one, two, three, four, or five amino acid residues directly following SEQ ID NO:210, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR1 that consists essentially of the amino acid sequence set forth in SEQ ID NO:210 include, without limitation, those set forth in Table 9.

TABLE 9
Exemplary CDR1s that consist essentially of the
amino acid sequence set forth in SEQ ID NO: 210.

| Sequence | SEQ ID NO: |
|---|---|
| NYYMS | 284 |
| NNYMS | 285 |
| NNYMH | 286 |
| NNYMG | 287 |
| NNYME | 288 |
| DYYMH | 289 |
| DYYMD | 290 |
| DYYMG | 291 |
| NYYMH | 292 |
| EYYMH | 293 |

As used herein, a “CDR2 that consists essentially of the amino acid sequence set forth in SEQ ID NO:211” is a CDR2 that has zero, one, or two amino acid substitutions within SEQ ID NO:211, that has zero, one, two, three, four, or five amino acid residues directly preceding SEQ ID NO:211, and/or that has zero, one, two, three, four, or five amino acid residues directly following SEQ ID NO:211, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR2 that consists essentially of the amino acid sequence set forth in SEQ ID NO:211 include, without limitation, those set forth in Table 10.

TABLE 10
Exemplary CDR2s that consist essentially of the
amino acid sequence set forth in SEQ ID NO: 211.

| Sequence | SEQ ID NO: |
|---|---|
| DINHSGSTNYNPSLKS | 294 |
| EINHSGSTNYNPSLKT | 295 |
| EIQHSGSTNYNPSLKS | 296 |
| EINHTGSTNYNPSLKS | 297 |
| EINHSGTTNYNPSLKS | 298 |
| EINHSGSTNYDPSLKS | 299 |
| EINHSGSTNYNPTLKS | 300 |
| EINHSGSTNYNPSLRS | 301 |
| EINHSGSTNYNPSLKG | 302 |
| EINHSGSTNYNPSLKA | 303 |

As used herein, a “CDR3 that consists essentially of the amino acid sequence set forth in SEQ ID NO:212” is a CDR3 that has zero, one, or two amino acid substitutions within SEQ ID NO:212, that has zero, one, two, three, four, or five amino acid residues directly preceding SEQ TD NO:212, and/or that has zero, one, two, three, four, or five amino acid residues directly following SEQ ID NO:212, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR3 that consists essentially of the amino acid sequence set forth in SEQ ID NO:212 include, without limitation, those set forth in Table 11.

TABLE 11
Exemplary CDR3s that consist essentially of the
amino acid sequence set forth in SEQ ID NO: 212.

| Sequence | SEQ ID NO: |
|---|---|
| EGEWYDAFDI | 304 |
| DGDWYDAFDI | 305 |
| EGDWYEAFDI | 306 |
| EGDWYDVFDI | 307 |
| EGDWYDAFEI | 308 |
| EGDWYDAFDL | 309 |
| EGEWYDAFEI | 310 |
| EGDWYEAFEI | 311 |
| DGEWYDAFDI | 312 |
| DGEWYEAFDI | 313 |

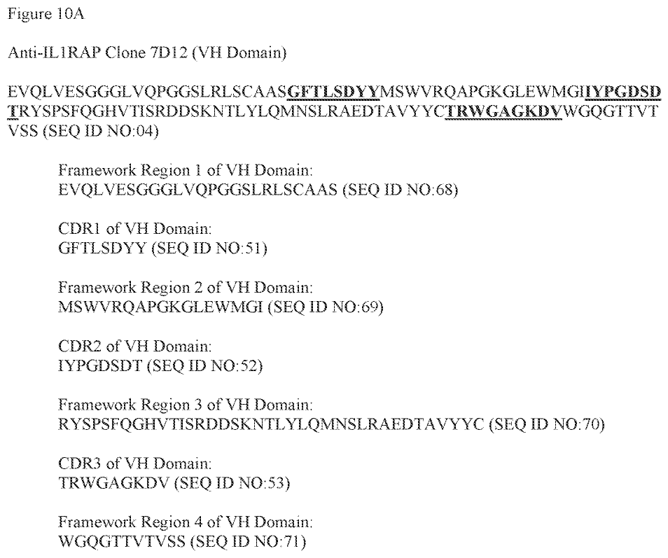

In another embodiment, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having a CDR1 having the amino acid sequence set forth in SEQ ID NO:51 (or a variant of SEQ ID NO:51 with one or two amino acid modifications), a CDR2 having the amino acid sequence set forth in SEQ ID NO:52 (or a variant of SEQ ID NO:52 with one or two amino acid modifications), and a CDR3 having the amino acid sequence set forth in SEQ ID NO:53 (or a variant of SEQ ID NO:53 with one or two amino acid modifications). An example of such an antibody domain having these CDRs and the ability to bind to an IL1RAP polypeptide (e.g., a human IL1RAP polypeptide) includes, without limitation, the VH domain set forth in FIG. 10A.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) and having a heavy chain variable domain having a CDR1 having the amino acid sequence set forth in SEQ ID NO:51 (or a variant of SEQ ID NO:51 with one or two amino acid modifications), a CDR2 having the amino acid sequence set forth in SEQ ID NO:52 (or a variant of SEQ ID NO:52 with one or two amino acid modifications), and a CDR3 having the amino acid sequence set forth in SEQ ID NO:53 (or a variant of SEQ ID NO:53 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:68 (or a variant of SEQ ID NO:68 with one, two, three, four, five, six, seven, eight, nine, ten, or more amino acid modifications), a framework region 2 having the amino acid sequence set forth in SEQ ID NO:69 (or a variant of SEQ ID NO:69 with one, two, three, four, five, six, seven, eight, nine, ten, or more amino acid modifications), a framework region 3 having the amino acid sequence set forth in SEQ ID NO:70 (or a variant of SEQ ID NO:70 with one, two, three, four, five, six, seven, eight, nine, ten, or more amino acid modifications), and a framework region 4 having the amino acid sequence set forth in SEQ ID NO:71 (or a variant of SEQ ID NO:71 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 10A can be designed to include framework regions as set forth in FIG. 10A or can be designed to include one or more framework regions from another antibody or antibody fragment. For example, an antibody domain (e.g., a VH domain) can be designed to include the three CDRs set forth in FIG. 10A and the framework regions set forth in FIG. 10A except that framework region 1 having the amino acid set forth in SEQ ID NO:68 is replaced with a framework region 1 having the amino acid set forth in SEQ ID NO:56, a framework region 1 having the amino acid set forth in SEQ ID NO:60, or a framework region 1 having the amino acid set forth in SEQ ID NO:64.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:04. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:04. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:04.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:04, provided that the heavy chain variable domain includes the amino acid sequences set forth in SEQ ID NOs:51, 52, and 53. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:04, provided that the heavy chain variable domain includes the amino acid sequences set forth in SEQ ID NOs:51, 52, and 53.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having the amino acid sequence set forth in SEQ ID NO:04 or the amino acid set forth in SEQ ID NO:04 with one, two, three, four, five, six, seven, eight, nine, or 10 amino acid modifications (e.g., amino acid substitutions, amino acid deletions, and/or amino acid additions). For example, an antibody domain (e.g., a VH domain) provided herein can have the ability to bind to an IL1RAP polypeptide (e.g., a human IL1RAP polypeptide) and can include a heavy chain variable domain having the amino acid sequence set forth in SEQ ID NO:04 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:51, 52, and 53.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain comprising (i) a CDR1 that comprises, consists essentially of, or consists of the amino acid sequence set forth in SEQ ID NO:51, (ii) a CDR2 that comprises, consists essentially of, or consists of the amino acid sequence set forth in SEQ ID NO:52, and (iii) a CDR3 that comprises, consists essentially of, or consists of the amino acid sequence set forth in SEQ ID NO:53.

As used herein, a “CDR1 that consists essentially of the amino acid sequence set forth in SEQ ID NO:51” is a CDR1 that has zero, one, or two amino acid substitutions within SEQ ID NO:51, that has zero, one, two, three, four, or five amino acid residues directly preceding SEQ ID NO:51, and/or that has zero, one, two, three, four, or five amino acid residues directly following SEQ ID NO:51, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide).

As used herein, a “CDR2 that consists essentially of the amino acid sequence set forth in SEQ ID NO:52” is a CDR2 that has zero, one, or two amino acid substitutions within SEQ ID NO:52, that has zero, one, two, three, four, or five amino acid residues directly preceding SEQ ID NO:52, and/or that has zero, one, two, three, four, or five amino acid residues directly following SEQ ID NO:52, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide).

As used herein, a “CDR3 that consists essentially of the amino acid sequence set forth in SEQ ID NO:53” is a CDR3 that has zero, one, or two amino acid substitutions within SEQ ID NO:53, that has zero, one, two, three, four, or five amino acid residues directly preceding SEQ ID NO:53, and/or that has zero, one, two, three, four, or five amino acid residues directly following SEQ ID NO:53, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide).

In another embodiment, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having a CDR1 having the amino acid sequence set forth in SEQ ID NO:217 (or a variant of SEQ ID NO:217 with one or two amino acid modifications), a CDR2 having the amino acid sequence set forth in SEQ ID NO:218 (or a variant of SEQ ID NO:218 with one or two amino acid modifications), and a CDR3 having the amino acid sequence set forth in SEQ ID NO:219 (or a variant of SEQ ID NO:219 with one or two amino acid modifications). An example of such an antibody domain having these CDRs and the ability to bind to an IL1RAP polypeptide (e.g., a human IL1RAP polypeptide) includes, without limitation, the VH domain set forth in FIG. 10B.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) and having a heavy chain variable domain having a CDR1 having the amino acid sequence set forth in SEQ ID NO:217 (or a variant of SEQ ID NO:217 with one or two amino acid modifications), a CDR2 having the amino acid sequence set forth in SEQ ID NO:218 (or a variant of SEQ ID NO:218 with one or two amino acid modifications), and a CDR3 having the amino acid sequence set forth in SEQ ID NO:219 (or a variant of SEQ ID NO:219 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:220 (or a variant of SEQ ID NO:220 with one, two, three, four, five, six, seven, eight, nine, ten, or more amino acid modifications), a framework region 2 having the amino acid sequence set forth in SEQ ID NO:221 (or a variant of SEQ ID NO:221 with one, two, three, four, five, six, seven, eight, nine, ten, or more amino acid modifications), a framework region 3 having the amino acid sequence set forth in SEQ ID NO:222 (or a variant of SEQ ID NO:222 with one, two, three, four, five, six, seven, eight, nine, ten, or more amino acid modifications), and a framework region 4 having the amino acid sequence set forth in SEQ ID NO:223 (or a variant of SEQ ID NO:223 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 10B can be designed to include framework regions as set forth in FIG. 10B or can be designed to include one or more framework regions from another antibody or antibody fragment. For example, an antibody domain (e.g., a VH domain) can be designed to include the three CDRs set forth in FIG. 10B and the framework regions set forth in FIG. 10B except that framework region 1 having the amino acid set forth in SEQ ID NO:220 is replaced with a framework region 1 having the amino acid set forth in SEQ ID NO:199, a framework region 1 having the amino acid set forth in SEQ ID NO:206, or a framework region 1 having the amino acid set forth in SEQ ID NO:213.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:04, provided that the heavy chain variable domain includes the amino acid sequences set forth in SEQ ID NOs:217, 218, and 219. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:04, provided that the heavy chain variable domain includes the amino acid sequences set forth in SEQ ID NOs:217, 218, and 219.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having the amino acid sequence set forth in SEQ ID NO:04 or the amino acid set forth in SEQ ID NO:04 with one, two, three, four, five, six, seven, eight, nine, or 10 amino acid modifications (e.g., amino acid substitutions, amino acid deletions, and/or amino acid additions). For example, an antibody domain (e.g., a VH domain) provided herein can have the ability to bind to an IL1RAP polypeptide (e.g., a human IL1RAP polypeptide) and can include a heavy chain variable domain having the amino acid sequence set forth in SEQ ID NO:04 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:217, 218, and 219.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain comprising (i) a CDR1 that comprises, consists essentially of, or consists of the amino acid sequence set forth in SEQ ID NO:217, (ii) a CDR2 that comprises, consists essentially of, or consists of the amino acid sequence set forth in SEQ ID NO:218, and (iii) a CDR3 that comprises, consists essentially of, or consists of the amino acid sequence set forth in SEQ ID NO:219. As used herein, a “CDR1 that consists essentially of the amino acid sequence set forth in SEQ ID NO:217” is a CDR1 that has zero, one, or two amino acid substitutions within SEQ ID NO:217, that has zero, one, two, three, four, or five amino acid residues directly preceding SEQ ID NO:217, and/or that has zero, one, two, three, four, or five amino acid residues directly following SEQ ID NO:217, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR1 that consists essentially of the amino acid sequence set forth in SEQ ID NO:217 include, without limitation, those set forth in Table 12.

TABLE 12
Exemplary CDR1s that consist essentially of the
amino acid sequence set forth in SEQ ID NO: 217.

| Sequence | SEQ ID NO: |
|---|---|
| NYYMS | 314 |
| NNYMS | 315 |
| NNYMH | 316 |
| NNYMG | 317 |
| NNYME | 318 |
| DYYMH | 319 |
| DYYMD | 320 |
| DYYMG | 321 |
| NYYMH | 322 |
| EYYMH | 323 |

As used herein, a “CDR2 that consists essentially of the amino acid sequence set forth in SEQ ID NO:218” is a CDR2 that has zero, one, or two amino acid substitutions within SEQ ID NO:218, that has zero, one, two, three, four, or five amino acid residues directly preceding SEQ ID NO:218, and/or that has zero, one, two, three, four, or five amino acid residues directly following SEQ ID NO:218, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR2 that consists essentially of the amino acid sequence set forth in SEQ ID NO:218 include, without limitation, those set forth in Table 13.

TABLE 13
Exemplary CDR2s that consist essentially
of the amino acid sequence set forth in
SEQ ID NO: 218.

| Sequence | SEQ ID NO: |
|---|---|
| IIYPGDSDTRYSPSFQS | 324 |
| IIYPGDSDTRYSPSFQA | 325 |
| IIYPGDSETRYSPSFQS | 326 |
| IIYPGDSETRYSPSFQG | 327 |
| IIYPGDTETRYSPSFQG | 328 |
| IIYPGDTDTRYSPSFQS | 329 |
| IIYPGDTDTRYTPSFQS | 330 |
| IIYPGDTDTRYTPSFQG | 331 |
| IIYPGDTDTRYTPSFNG | 332 |
| IIYPGETDTRYTPSFNG | 333 |

As used herein, a “CDR3 that consists essentially of the amino acid sequence set forth in SEQ ID NO:219” is a CDR3 that has zero, one, or two amino acid substitutions within SEQ ID NO:219, that has zero, one, two, three, four, or five amino acid residues directly preceding SEQ ID NO:219, and/or that has zero, one, two, three, four, or five amino acid residues directly following SEQ ID NO:219, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR3 that consists essentially of the amino acid sequence set forth in SEQ ID NO:219 include, without limitation, those set forth in Table 14.

TABLE 14
Exemplary CDR3s that consist essentially
of the amino acid sequence set forth in
SEQ ID NO: 219.

| Sequence | SEQ ID NO: |
|---|---|
| WSAGKDV | 334 |
| WGASKDV | 335 |
| WGAGKDL | 336 |
| WGTGKDV | 337 |
| WGAGKEV | 338 |
| WGAGKEL | 339 |
| WTAGKEV | 340 |
| WGATKDL | 341 |
| WGATKEV | 342 |
| WGAGKDA | 343 |

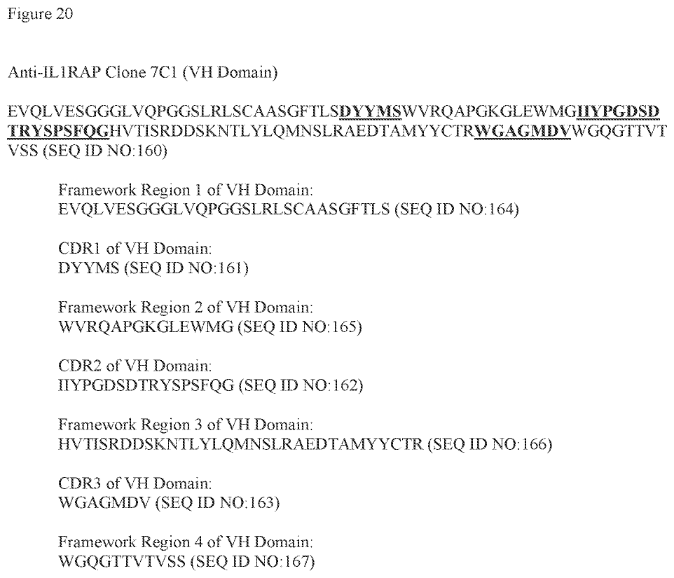

In another embodiment, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having a CDR1 having the amino acid sequence set forth in SEQ ID NO:161 (or a variant of SEQ ID NO:161 with one or two amino acid modifications), a CDR2 having the amino acid sequence set forth in SEQ ID NO:162 (or a variant of SEQ ID NO:162 with one or two amino acid modifications), and a CDR3 having the amino acid sequence set forth in SEQ ID NO:163 (or a variant of SEQ ID NO:163 with one or two amino acid modifications). An example of such an antibody domain having these CDRs and the ability to bind to an IL1RAP polypeptide (e.g., a human IL1RAP polypeptide) includes, without limitation, the VH domain set forth in FIG. 20.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) and having a heavy chain variable domain having a CDR1 having the amino acid sequence set forth in SEQ ID NO:161 (or a variant of SEQ ID NO:161 with one or two amino acid modifications), a CDR2 having the amino acid sequence set forth in SEQ ID NO:162 (or a variant of SEQ ID NO:162 with one or two amino acid modifications), and a CDR3 having the amino acid sequence set forth in SEQ ID NO:163 (or a variant of SEQ ID NO:163 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:164 (or a variant of SEQ ID NO:164 with one, two, three, four, five, six, seven, eight, nine, ten, or more amino acid modifications), a framework region 2 having the amino acid sequence set forth in SEQ ID NO:165 (or a variant of SEQ ID NO:165 with one, two, three, four, five, six, seven, eight, nine, ten, or more amino acid modifications), a framework region 3 having the amino acid sequence set forth in SEQ ID NO:166 (or a variant of SEQ ID NO:166 with one, two, three, four, five, six, seven, eight, nine, ten, or more amino acid modifications), and a framework region 4 having the amino acid sequence set forth in SEQ ID NO:167 (or a variant of SEQ ID NO:167 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 20 can be designed to include framework regions as set forth in FIG. 20 or can be designed to include one or more framework regions from another antibody or antibody fragment. For example, an antibody domain (e.g., a VH domain) can be designed to include the three CDRs set forth in FIG. 20 and the framework regions set forth in FIG. 20 except that framework region 1 having the amino acid set forth in SEQ ID NO:164 is replaced with a framework region 1 having the amino acid set forth in SEQ ID NO:199, a framework region 1 having the amino acid set forth in SEQ ID NO:206, a framework region 1 having the amino acid set forth in SEQ ID NO:213, or a framework region 1 having the amino acid set forth in SEQ ID NO:220.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:160. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:160. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:160.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO: 160, provided that the heavy chain variable domain includes the amino acid sequences set forth in SEQ ID NOs:161, 162, and 163. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:160, provided that the heavy chain variable domain includes the amino acid sequences set forth in SEQ ID NOs:161, 162, and 163.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having the amino acid sequence set forth in SEQ ID NO: 160 or the amino acid set forth in SEQ ID NO: 160 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to an IL1RAP polypeptide (e.g., a human IL1RAP polypeptide) and can include a heavy chain variable domain having the amino acid sequence set forth in SEQ ID NO:160 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:161, 162, and 163.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain comprising (i) a CDR1 that comprises, consists essentially of, or consists of the amino acid sequence set forth in SEQ ID NO: 161, (ii) a CDR2 that comprises, consists essentially of, or consists of the amino acid sequence set forth in SEQ ID NO:162, and (iii) a CDR3 that comprises, consists essentially of, or consists of the amino acid sequence set forth in SEQ ID NO:163. As used herein, a “CDR1 that consists essentially of the amino acid sequence set forth in SEQ ID NO:161” is a CDR1 that has zero, one, or two amino acid substitutions within SEQ ID NO:161, that has zero, one, two, three, four, or five amino acid residues directly preceding SEQ ID NO:161, and/or that has zero, one, two, three, four, or five amino acid residues directly following SEQ ID NO:161, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR1 that consists essentially of the amino acid sequence set forth in SEQ ID NO:161 include, without limitation, those set forth in Table 16.

TABLE 16
Exemplary CDR1s that consist essentially
of the amino acid sequence set forth in
SEQ ID NO: 161.

| Sequence | SEQ ID NO: |
|---|---|
| NYYMS | 344 |
| NNYMS | 345 |
| NNYMH | 346 |
| NNYMG | 347 |
| NNYME | 348 |
| DYYMH | 349 |
| DYYMD | 350 |
| DYYMG | 351 |
| NYYMH | 352 |
| EYYMH | 353 |

As used herein, a “CDR2 that consists essentially of the amino acid sequence set forth in SEQ ID NO:162” is a CDR2 that has zero, one, or two amino acid substitutions within SEQ ID NO:162, that has zero, one, two, three, four, or five amino acid residues directly preceding SEQ ID NO:162, and/or that has zero, one, two, three, four, or five amino acid residues directly following SEQ ID NO:162, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR2 that consists essentially of the amino acid sequence set forth in SEQ ID NO:162 include, without limitation, those set forth in Table 17.

TABLE 17
Exemplary CDR2s that consist essentially
of the amino acid sequence set forth in
SEQ ID NO: 162.

| Sequence | SEQ ID NO: |
|---|---|
| IIYPGDSDTRYSPSFQS | 354 |
| IIYPGDSDTRYSPSFQA | 355 |
| IIYPGDSETRYSPSFQS | 356 |
| IIYPGDSETRYSPSFQG | 357 |
| IIYPGDTETRYSPSFQG | 358 |
| IIYPGDTDTRYSPSFQS | 359 |
| IIYPGDTDTRYTPSFQS | 360 |
| IIYPGDTDTRYTPSFQG | 361 |
| IIYPGDTDTRYTPSFNG | 362 |
| IIYPGETDTRYTPSFNG | 363 |

As used herein, a “CDR3 that consists essentially of the amino acid sequence set forth in SEQ ID NO:163” is a CDR3 that has zero, one, or two amino acid substitutions within SEQ ID NO:163, that has zero, one, two, three, four, or five amino acid residues directly preceding SEQ ID NO:163, and/or that has zero, one, two, three, four, or five amino acid residues directly following SEQ ID NO:163, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR3 that consists essentially of the amino acid sequence set forth in SEQ ID NO:163 include, without limitation, those set forth in Table 18.

TABLE 18
Exemplary CDR3s that consist essentially
of the amino acid sequence set forth in
SEQ ID NO: 163.

| Sequence | SEQ ID NO: |
|---|---|
| WSAGMDV | 364 |
| WGASMDV | 365 |
| WGAGMDL | 366 |
| WGTGMDV | 367 |
| WGAGMEV | 368 |
| WGAGMEL | 369 |
| WTAGMEV | 370 |
| WGATMDL | 371 |
| WGATMEV | 372 |
| WGAGMDA | 373 |

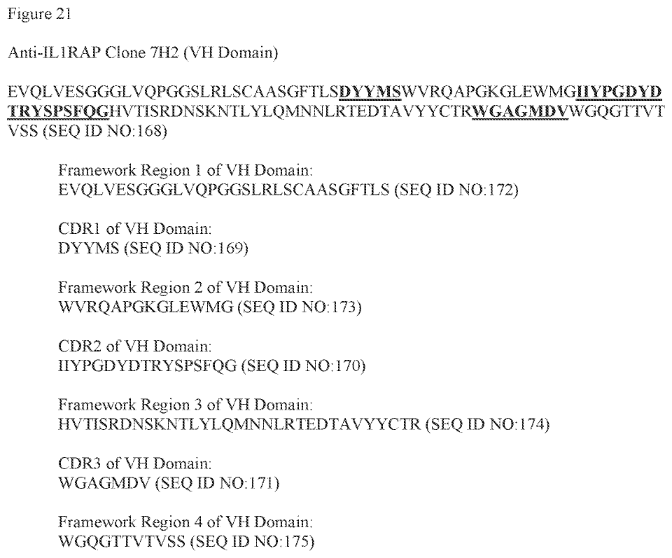

In another embodiment, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having a CDR1 having the amino acid sequence set forth in SEQ ID NO:169 (or a variant of SEQ ID NO:169 with one or two amino acid modifications), a CDR2 having the amino acid sequence set forth in SEQ ID NO: 170 (or a variant of SEQ ID NO:170 with one or two amino acid modifications), and a CDR3 having the amino acid sequence set forth in SEQ ID NO:171 (or a variant of SEQ ID NO:171 with one or two amino acid modifications). An example of such an antibody domain having these CDRs and the ability to bind to an IL1RAP polypeptide (e.g., a human IL1RAP polypeptide) includes, without limitation, the VH domain set forth in FIG. 21.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) and having a heavy chain variable domain having a CDR1 having the amino acid sequence set forth in SEQ ID NO:169 (or a variant of SEQ ID NO:169 with one or two amino acid modifications), a CDR2 having the amino acid sequence set forth in SEQ ID NO: 170 (or a variant of SEQ ID NO:170 with one or two amino acid modifications), and a CDR3 having the amino acid sequence set forth in SEQ ID NO:171 (or a variant of SEQ ID NO:171 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:172 (or a variant of SEQ ID NO:172 with one, two, three, four, five, six, seven, eight, nine, ten, or more amino acid modifications), a framework region 2 having the amino acid sequence set forth in SEQ ID NO:173 (or a variant of SEQ ID NO:173 with one, two, three, four, five, six, seven, eight, nine, ten, or more amino acid modifications), a framework region 3 having the amino acid sequence set forth in SEQ ID NO:174 (or a variant of SEQ ID NO:174 with one, two, three, four, five, six, seven, eight, nine, ten, or more amino acid modifications), and a framework region 4 having the amino acid sequence set forth in SEQ ID NO:175 (or a variant of SEQ ID NO:175 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 21 can be designed to include framework regions as set forth in FIG. 21 or can be designed to include one or more framework regions from another antibody or antibody fragment. For example, an antibody domain (e.g., a VH domain) can be designed to include the three CDRs set forth in FIG. 21 and the framework regions set forth in FIG. 21 except that framework region 1 having the amino acid set forth in SEQ ID NO:172 is replaced with a framework region 1 having the amino acid set forth in SEQ ID NO:199, a framework region 1 having the amino acid set forth in SEQ ID NO:206, a framework region 1 having the amino acid set forth in SEQ ID NO:213, or a framework region 1 having the amino acid set forth in SEQ ID NO:220.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:168. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:168. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO: 168.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO: 168, provided that the heavy chain variable domain includes the amino acid sequences set forth in SEQ ID NOs:169, 170, and 171. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:168, provided that the heavy chain variable domain includes the amino acid sequences set forth in SEQ ID NOs:169, 170, and 171.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having the amino acid sequence set forth in SEQ ID NO: 168 or the amino acid set forth in SEQ ID NO: 168 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to an IL1RAP polypeptide (e.g., a human IL1RAP polypeptide) and can include a heavy chain variable domain having the amino acid sequence set forth in SEQ ID NO:168 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:169, 170, and 171.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain comprising (i) a CDR1 that comprises, consists essentially of, or consists of the amino acid sequence set forth in SEQ ID NO: 169, (ii) a CDR2 that comprises, consists essentially of, or consists of the amino acid sequence set forth in SEQ ID NO:170, and (iii) a CDR3 that comprises, consists essentially of, or consists of the amino acid sequence set forth in SEQ ID NO:171. As used herein, a “CDR1 that consists essentially of the amino acid sequence set forth in SEQ ID NO:169” is a CDR1 that has zero, one, or two amino acid substitutions within SEQ ID NO:169, that has zero, one, two, three, four, or five amino acid residues directly preceding SEQ ID NO:169, and/or that has zero, one, two, three, four, or five amino acid residues directly following SEQ ID NO:169, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR1 that consists essentially of the amino acid sequence set forth in SEQ ID NO:169 include, without limitation, those set forth in Table 19.

TABLE 19
Exemplary CDR1s that consist essentially
of the amino acid sequence set forth in
SEQ ID NO: 169.

| Sequence | SEQ ID NO: |
|---|---|
| NYYMS | 374 |
| NNYMS | 375 |
| NNYMH | 376 |
| NNYMG | 377 |
| NNYME | 378 |
| DYYMH | 379 |
| DYYMD | 380 |
| DYYMG | 381 |
| NYYMH | 382 |
| EYYMH | 383 |

As used herein, a “CDR2 that consists essentially of the amino acid sequence set forth in SEQ ID NO:170” is a CDR2 that has zero, one, or two amino acid substitutions within SEQ ID NO:170, that has zero, one, two, three, four, or five amino acid residues directly preceding SEQ ID NO:170, and/or that has zero, one, two, three, four, or five amino acid residues directly following SEQ ID NO:170, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR2 that consists essentially of the amino acid sequence set forth in SEQ ID NO:170 include, without limitation, those set forth in Table 20.

TABLE 20
Exemplary CDR2s that consist essentially
of the amino acid sequence set forth in
SEQ ID NO: 170.

| Sequence | SEQ ID NO: |
|---|---|
| IIYPGDYDTRYSPSFQS | 384 |
| IIYPGDYDTRYSPSFQA | 385 |
| IIYPGDYETRYSPSFQS | 386 |
| IIYPGDYETRYSPSFQG | 387 |
| IIYPGDTETRYSPSFQG | 388 |
| IIYPGDTDTRYSPSFQS | 389 |
| IIYPGDTDTRYTPSFQS | 390 |
| IIYPGDTDTRYTPSFQG | 391 |
| IIYPGDTDTRYTPSFNG | 392 |
| IIYPGETDTRYTPSFNG | 393 |

As used herein, a “CDR3 that consists essentially of the amino acid sequence set forth in SEQ ID NO:171” is a CDR3 that has zero, one, or two amino acid substitutions within SEQ ID NO:171, that has zero, one, two, three, four, or five amino acid residues directly preceding SEQ TD NO:171, and/or that has zero, one, two, three, four, or five amino acid residues directly following SEQ ID NO:171, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR3 that consists essentially of the amino acid sequence set forth in SEQ ID NO: 171 include, without limitation, those set forth in Table 21.

TABLE 21
Exemplary CDR3s that consist essentially
of the amino acid sequence set forth in
SEQ ID NO: 171.

| Sequence | SEQ ID NO: |
|---|---|
| WSAGMDV | 394 |
| WGASMDV | 395 |
| WGAGMDL | 396 |
| WGTGMDV | 397 |
| WGAGMEV | 398 |
| WGAGMEL | 399 |
| WTAGMEV | 400 |
| WGATMDL | 401 |
| WGATMEV | 402 |
| WGAGMDA | 403 |

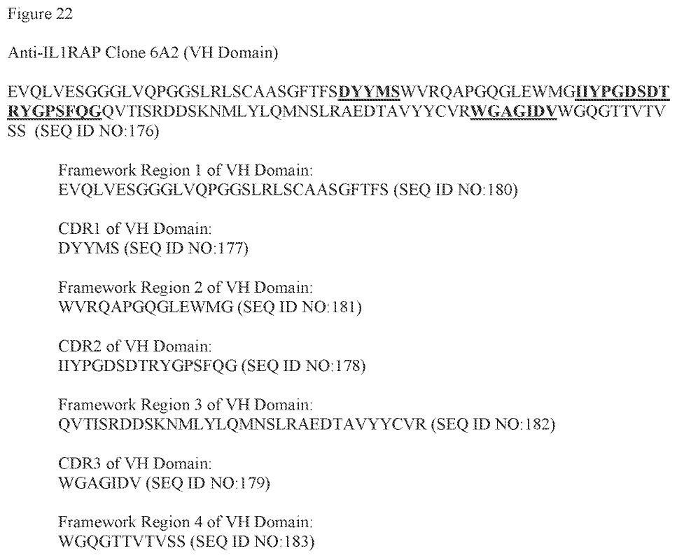

In another embodiment, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having a CDR1 having the amino acid sequence set forth in SEQ ID NO:177 (or a variant of SEQ ID NO:177 with one or two amino acid modifications), a CDR2 having the amino acid sequence set forth in SEQ ID NO: 178 (or a variant of SEQ ID NO:178 with one or two amino acid modifications), and a CDR3 having the amino acid sequence set forth in SEQ ID NO:179 (or a variant of SEQ ID NO:179 with one or two amino acid modifications). An example of such an antibody domain having these CDRs and the ability to bind to an IL1RAP polypeptide (e.g., a human IL1RAP polypeptide) includes, without limitation, the VH domain set forth in FIG. 22.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) and having a heavy chain variable domain having a CDR1 having the amino acid sequence set forth in SEQ ID NO:177 (or a variant of SEQ ID NO:177 with one or two amino acid modifications), a CDR2 having the amino acid sequence set forth in SEQ ID NO: 178 (or a variant of SEQ ID NO:178 with one or two amino acid modifications), and a CDR3 having the amino acid sequence set forth in SEQ ID NO:179 (or a variant of SEQ ID NO:179 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:180 (or a variant of SEQ ID NO:180 with one, two, three, four, five, six, seven, eight, nine, ten, or more amino acid modifications), a framework region 2 having the amino acid sequence set forth in SEQ ID NO:181 (or a variant of SEQ ID NO:181 with one, two, three, four, five, six, seven, eight, nine, ten, or more amino acid modifications), a framework region 3 having the amino acid sequence set forth in SEQ ID NO:182 (or a variant of SEQ ID NO:182 with one, two, three, four, five, six, seven, eight, nine, ten, or more amino acid modifications), and a framework region 4 having the amino acid sequence set forth in SEQ ID NO:183 (or a variant of SEQ ID NO:183 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 22 can be designed to include framework regions as set forth in FIG. 22 or can be designed to include one or more framework regions from another antibody or antibody fragment. For example, an antibody domain (e.g., a VH domain) can be designed to include the three CDRs set forth in FIG. 22 and the framework regions set forth in FIG. 22 except that framework region 1 having the amino acid set forth in SEQ ID NO:180 is replaced with a framework region 1 having the amino acid set forth in SEQ ID NO:199, a framework region 1 having the amino acid set forth in SEQ ID NO:206, a framework region 1 having the amino acid set forth in SEQ ID NO:213, or a framework region 1 having the amino acid set forth in SEQ ID NO:220.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:176. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:176. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:176.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO: 176, provided that the heavy chain variable domain includes the amino acid sequences set forth in SEQ ID NOs:177, 178, and 179. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:176, provided that the heavy chain variable domain includes the amino acid sequences set forth in SEQ ID NOs:177, 178, and 179.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having the amino acid sequence set forth in SEQ ID NO: 176 or the amino acid set forth in SEQ ID NO: 176 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to an IL1RAP polypeptide (e.g., a human IL1RAP polypeptide) and can include a heavy chain variable domain having the amino acid sequence set forth in SEQ ID NO:176 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:177, 178, and 179.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain comprising (i) a CDR1 that comprises, consists essentially of, or consists of the amino acid sequence set forth in SEQ ID NO: 177, (ii) a CDR2 that comprises, consists essentially of, or consists of the amino acid sequence set forth in SEQ ID NO:178, and (iii) a CDR3 that comprises, consists essentially of, or consists of the amino acid sequence set forth in SEQ ID NO:179. As used herein, a “CDR1 that consists essentially of the amino acid sequence set forth in SEQ ID NO:177” is a CDR1 that has zero, one, or two amino acid substitutions within SEQ ID NO:177, that has zero, one, two, three, four, or five amino acid residues directly preceding SEQ ID NO:177, and/or that has zero, one, two, three, four, or five amino acid residues directly following SEQ ID NO:177, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR1 that consists essentially of the amino acid sequence set forth in SEQ ID NO:177 include, without limitation, those set forth in Table 22.

TABLE 22
Exemplary CDR1s that consist essentially
of the amino acid sequence set forth in
SEQ ID NO: 177.

| Sequence | SEQ ID NO: |
|---|---|
| NYYMS | 404 |
| NNYMS | 405 |
| NNYMH | 406 |
| NNYMG | 407 |
| NNYME | 408 |
| DYYMH | 409 |
| DYYMD | 410 |
| DYYMG | 411 |
| NYYMH | 412 |
| EYYMH | 413 |

As used herein, a “CDR2 that consists essentially of the amino acid sequence set forth in SEQ ID NO:178” is a CDR2 that has zero, one, or two amino acid substitutions within SEQ ID NO: 178, that has zero, one, two, three, four, or five amino acid residues directly preceding SEQ TD NO: 178, and/or that has zero, one, two, three, four, or five amino acid residues directly following SEQ ID NO: 178, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR2 that consists essentially of the amino acid sequence set forth in SEQ ID NO: 178 include, without limitation, those set forth in Table 23.

TABLE 23
Exemplary CDR2s that consist essentially
of the amino acid sequence set forth in
SEQ ID NO: 178.

| Sequence | SEQ ID NO: |
|---|---|
| IIYPGDSDTRYGPSFQS | 414 |
| IIYPGDSDTRYGPSFQA | 415 |
| IIYPGDSETRYGPSFQS | 416 |
| IIYPGDSETRYGPSFQG | 417 |
| IIYPGDTETRYGPSFQG | 418 |
| IIYPGDTDTRYGPSFQS | 419 |
| IIYPGDTDTRYTPSFQS | 420 |
| IIYPGDTDTRYTPSFQG | 421 |
| IIYPGDTDTRYTPSFNG | 422 |
| IIYPGETDTRYTPSFNG | 423 |

As used herein, a “CDR3 that consists essentially of the amino acid sequence set forth in SEQ ID NO: 179” is a CDR3 that has zero, one, or two amino acid substitutions within SEQ ID NO:179, that has zero, one, two, three, four, or five amino acid residues directly preceding SEQ ID NO:179, and/or that has zero, one, two, three, four, or five amino acid residues directly following SEQ ID NO:179, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR3 that consists essentially of the amino acid sequence set forth in SEQ ID NO:179 include, without limitation, those set forth in Table 24.

TABLE 24
Exemplary CDR3s that consist essentially
of the amino acid sequence set forth in
SEQ ID NO: 179.

| Sequence | SEQ ID NO: |
|---|---|
| WSAGIDV | 424 |
| WGASIDV | 425 |
| WGAGIDL | 426 |
| WGTGIDV | 427 |
| WGAGIEV | 428 |
| WGAGIEL | 429 |
| WTAGIEV | 430 |
| WGATIDL | 431 |
| WGATIEV | 432 |
| WGAGIDA | 433 |

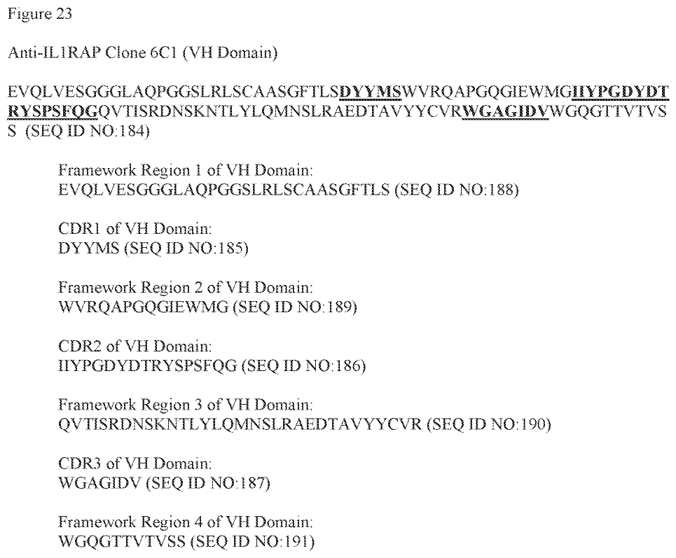

In another embodiment, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having a CDR1 having the amino acid sequence set forth in SEQ ID NO:185 (or a variant of SEQ ID NO:185 with one or two amino acid modifications), a CDR2 having the amino acid sequence set forth in SEQ ID NO: 186 (or a variant of SEQ ID NO:186 with one or two amino acid modifications), and a CDR3 having the amino acid sequence set forth in SEQ ID NO:187 (or a variant of SEQ ID NO:187 with one or two amino acid modifications). An example of such an antibody domain having these CDRs and the ability to bind to an IL1RAP polypeptide (e.g., a human IL1RAP polypeptide) includes, without limitation, the VH domain set forth in FIG. 23.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) and having a heavy chain variable domain having a CDR1 having the amino acid sequence set forth in SEQ ID NO:185 (or a variant of SEQ ID NO:185 with one or two amino acid modifications), a CDR2 having the amino acid sequence set forth in SEQ ID NO:186 (or a variant of SEQ ID NO:186 with one or two amino acid modifications), and a CDR3 having the amino acid sequence set forth in SEQ ID NO:187 (or a variant of SEQ ID NO:187 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:188 (or a variant of SEQ ID NO:188 with one, two, three, four, five, six, seven, eight, nine, ten, or more amino acid modifications), a framework region 2 having the amino acid sequence set forth in SEQ ID NO:189 (or a variant of SEQ ID NO:189 with one, two, three, four, five, six, seven, eight, nine, ten, or more amino acid modifications), a framework region 3 having the amino acid sequence set forth in SEQ ID NO:190 (or a variant of SEQ ID NO:190 with one, two, three, four, five, six, seven, eight, nine, ten, or more amino acid modifications), and a framework region 4 having the amino acid sequence set forth in SEQ ID NO:191 (or a variant of SEQ ID NO: 191 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 23 can be designed to include framework regions as set forth in FIG. 23 or can be designed to include one or more framework regions from another antibody or antibody fragment. For example, an antibody domain (e.g., a VH domain) can be designed to include the three CDRs set forth in FIG. 23 and the framework regions set forth in FIG. 23 except that framework region 1 having the amino acid set forth in SEQ ID NO:188 is replaced with a framework region 1 having the amino acid set forth in SEQ ID NO:199, a framework region 1 having the amino acid set forth in SEQ ID NO:206, a framework region 1 having the amino acid set forth in SEQ ID NO:213, or a framework region 1 having the amino acid set forth in SEQ ID NO:220.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO:184. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:184. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:184.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain that includes an amino acid sequence having at least 90 percent identity to the amino acid sequence set forth in SEQ ID NO: 184, provided that the heavy chain variable domain includes the amino acid sequences set forth in SEQ ID NOs:185, 186, and 187. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:184, provided that the heavy chain variable domain includes the amino acid sequences set forth in SEQ ID NOs:185, 186, and 187.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain having the amino acid sequence set forth in SEQ ID NO: 184 or the amino acid set forth in SEQ ID NO: 184 with one, two, three, four, five, six, seven, eight, nine, or 10 amino acid modifications (e.g., amino acid substitutions, amino acid deletions, and/or amino acid additions). For example, an antibody domain (e.g., a VH domain) provided herein can have the ability to bind to an IL1RAP polypeptide (e.g., a human IL1RAP polypeptide) and can include a heavy chain variable domain having the amino acid sequence set forth in SEQ ID NO:184 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:185, 186, and 187.

In some cases, a binder (e.g., an antibody, antigen binding fragment, antibody domain, a CAR, a cell engager, and/or an ADC) provided herein having the ability to bind to an IL1RAP polypeptide (e.g., a human IL1RAP polypeptide) can include a heavy chain variable domain comprising (i) a CDR1 that comprises, consists essentially of, or consists of the amino acid sequence set forth in SEQ ID NO: 185, (ii) a CDR2 that comprises, consists essentially of, or consists of the amino acid sequence set forth in SEQ ID NO:186, and (iii) a CDR3 that comprises, consists essentially of, or consists of the amino acid sequence set forth in SEQ ID NO:187. As used herein, a “CDR1 that consists essentially of the amino acid sequence set forth in SEQ ID NO:185” is a CDR1 that has zero, one, or two amino acid substitutions within SEQ ID NO:185, that has zero, one, two, three, four, or five amino acid residues directly preceding SEQ ID NO:185, and/or that has zero, one, two, three, four, or five amino acid residues directly following SEQ ID NO:185, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR1 that consists essentially of the amino acid sequence set forth in SEQ ID NO:185 include, without limitation, those set forth in Table 25.

TABLE 25
Exemplary CDR1s that consist essentially
of the amino acid sequence set forth in
SEQ ID NO: 185.

| Sequence | SEQ ID NO: |
|---|---|
| NYYMS | 434 |
| NNYMS | 435 |
| NNYMH | 436 |
| NNYMG | 437 |
| NNYME | 438 |
| DYYMH | 439 |
| DYYMD | 440 |
| DYYMG | 441 |
| NYYMH | 442 |
| EYYMH | 443 |

As used herein, a “CDR2 that consists essentially of the amino acid sequence set forth in SEQ ID NO:186” is a CDR2 that has zero, one, or two amino acid substitutions within SEQ ID NO:186, that has zero, one, two, three, four, or five amino acid residues directly preceding SEQ ID NO:186, and/or that has zero, one, two, three, four, or five amino acid residues directly following SEQ ID NO:186, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR2 that consists essentially of the amino acid sequence set forth in SEQ ID NO:186 include, without limitation, those set forth in Table 26.

TABLE 26
Exemplary CDR2s that consist essentially
of the amino acid sequence set forth in
SEQ ID NO: 186.

| Sequence | SEQ ID NO: |
|---|---|
| IIYPGDYDTRYGPSFQS | 444 |
| IIYPGDYDTRYGPSFQA | 445 |
| IIYPGDYETRYGPSFQS | 446 |
| IIYPGDYETRYGPSFQG | 447 |
| IIYPGDTETRYGPSFQG | 448 |
| IIYPGDTDTRYGPSFQS | 449 |
| IIYPGDTDTRYTPSFQS | 450 |
| IIYPGDTDTRYTPSFQG | 451 |
| IIYPGDTDTRYTPSFNG | 452 |
| IIYPGETDTRYTPSFNG | 453 |

As used herein, a “CDR3 that consists essentially of the amino acid sequence set forth in SEQ ID NO:187” is a CDR3 that has zero, one, or two amino acid substitutions within SEQ ID NO:187, that has zero, one, two, three, four, or five amino acid residues directly preceding SEQ ID NO:187, and/or that has zero, one, two, three, four, or five amino acid residues directly following SEQ ID NO:187, provided that the binder (e.g., an antibody, antigen binding fragment, antibody domain, a CAR, a cell engager, and/or an ADC) maintains its basic ability to bind to an IL1RAP polypeptide (e.g., a human IL1RAP polypeptide). Examples of a CDR3 that consists essentially of the amino acid sequence set forth in SEQ ID NO:187 include, without limitation, those set forth in Table 27.

TABLE 27
Exemplary CDR3s that consist essentially
of the amino acid sequence set forth in
SEQ ID NO: 187.

| Sequence | SEQ ID NO: |
|---|---|
| WSAGIDV | 454 |
| WGASIDV | 455 |
| WGAGIDL | 456 |
| WGTGIDV | 457 |
| WGAGIEV | 458 |
| WGAGIEL | 459 |
| WTAGIEV | 460 |
| WGATIDL | 461 |
| WGATIEV | 462 |
| WGAGIDA | 463 |

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a binder (e.g., an antibody, antigen binding fragment, antibody domain, a CAR, a cell engager, and/or an ADC) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr. Opin. Biotechnol. 3: 348-354 (1992). Both naturally occurring and non-naturally occurring amino acids (e.g., artificially-derivatized amino acids) can be used to generate an amino acid sequence variant provided herein.

A representative number of binders (e.g., antibodies, antigen binding fragments, and/or antibody domains) having the ability to bind to an IL1RAP polypeptide (e.g., a human IL1RAP polypeptide) are further described in Table 15.

TABLE 15
Representative number of binders.

| | SEQ ID NOs of Heavy | SEQ ID NOs of Heavy | |
|---|---|---|---|
| | Chain Variable | Chain Variable | |
| | Domain/Region | Domain/Region | SEQ ID NO of |
| Clone (Antibody type) | CDRs | Framework Regions | VH Domain |
| 3A7 (VH domain) | 42, 43, 44 (Alternative) | 56, 57, 58, 59 (Alternative) | 01 |
| 4G6 (VH domain) | 45, 46, 47 (Alternative) | 60, 61, 62, 63 (Alternative) | 02 |
| 3C5 (VH domain) | 48, 49, 50 (Alternative) | 64, 65, 66, 67 (Alternative) | 03 |
| 7D12 (VH domain) | 51, 52, 53 (Alternative) | 68, 69, 70, 71 (Alternative) | 04 |
| 3A7 (VH domain) | 196, 197, 198 | 199, 200, 201, 202 | 01 |
| 4G6 (VH domain) | 203, 204, 205 | 206, 207, 208, 209 | 02 |
| 3C5 (VH domain) | 210, 211, 212 | 213, 214, 215, 216 | 03 |
| 7D12 (VH domain) | 217, 218, 219 | 220, 221, 222, 223 | 04 |
| 7C1 (VH domain) | 161, 162, 163 | 164, 165, 166, 167 | 160 |
| 7H2 (VH domain) | 169, 170, 171 | 172, 173, 174, 175 | 168 |
| 6A2 (VH domain) | 177, 178, 179 | 180, 181, 182, 183 | 176 |
| 6C1 (VH domain) | 185, 186, 187 | 188, 189, 190, 191 | 184 |

When designing a single chain antibody (e.g., a scFv) having a heavy chain variable domain and a light chain variable domain, the two regions can be directly connected or can be connected using any appropriate linker sequence. For example, a heavy chain variable domain having the CDRs of SEQ ID NOs:42-44; SEQ ID NOs:196-198; SEQ ID NOs:45-47; SEQ ID NOs:203-205; SEQ ID NOs:48-50; SEQ ID NOs:210-212; SEQ ID NOs:51-53; SEQ ID NOs:217-219; SEQ ID NOs:161-163; SEQ ID NOs:169-171; SEQ ID NOs:177-179; or SEQ ID NOs:185-187 can be directly connected to a light chain variable domain via a linker sequence. Examples of linker sequences that can be used to connect a heavy chain variable domain and a light chain variable domain to create a scFv include, without limitation, those linkers set forth in FIG. 11 or FIG. 14.

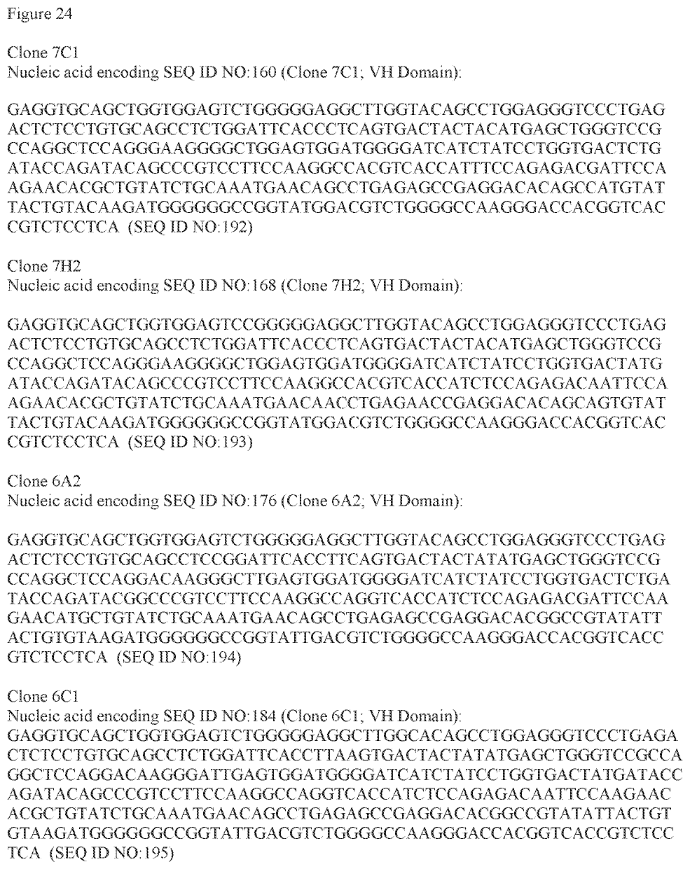

The binders (e.g., antibodies, antigen binding fragments, antibody domains, CARs, cell engagers, and/or ADCs) provided herein can be produced using any appropriate method. For example, the binders (e.g., antibodies, antigen binding fragments, antibody domains, CARs, and/or cell engagers) provided herein can be produced in recombinant host cells. For example, a nucleic acid encoding a binder (e.g., an antibody, antigen binding fragment, antibody domain, CAR, and/or cell engager) provided herein can be constructed, introduced into an expression vector, and expressed in suitable host cells. Table 2 and FIG. 24 include examples of nucleic acid sequences that encode exemplary binders (e.g., antibody domains) described herein. In some cases, a binder (e.g., an antibody, antigen binding fragment, antibody domain, CAR, and/or cell engager) provided herein can be recombinantly produced in prokaryotic hosts such as E. coli, Bacillus brevis, Bacillus subtilis, Bacillus megaterium, Lactobacillus zeae casei, or Lactobacillus paracasei. A binder (e.g., an antibody, antigen binding fragment, antibody domain, CAR, and/or cell engager) provided herein also can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T. reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NSO cells, baby hamster kidney (BHK) cells, or human embryonic kidney cell line HEK293). See, for example, the Frenzel et al. reference (FrontImmunol., 4:217 (2013)).

In some cases, an antigen binding fragment or antibody domain provided herein can be produced by proteolytic digestion of an intact antibody. For example, an antigen binding fragment can be obtained by treating an antibody with an enzyme such as papain or pepsin. Papain digestion of whole antibodies can be used to produce F(ab)₂ or Fab fragments, while pepsin digestion of whole antibodies can be used to produce F(ab′)2 or Fab′ fragments.

In some cases, a binder (e.g., an antibody, antigen binding fragment, antibody domain, CAR, cell engager, and/or ADC) provided herein can be substantially pure. The term “substantially pure” as used herein with reference to a binder (e.g., an antibody, antigen binding fragment, antibody domain, CAR, cell engager, and/or ADC) refers to the binder (e.g., an antibody, antigen binding fragment, antibody domain, CAR, cell engager, and/or ADC) as being substantially free of other polypeptides, lipids, carbohydrates, and nucleic acid with which it is naturally associated. Thus, a substantially pure binder (e.g., an antibody, antigen binding fragment, antibody domain, CAR, cell engager, and/or ADC) provided herein is any binder (e.g., an antibody, antigen binding fragment, antibody domain, CAR, cell engager, and/or ADC) that is removed from its natural environment and is at least 60 percent pure. A substantially pure binder (e.g., an antibody, antigen binding fragment, antibody domain, CAR, cell engager, and/or ADC) provided herein can be at least about 65, 70, 75, 80, 85, 90, 95, or 99 percent pure.

Some embodiments provided herein also include bispecific binders (e.g., bispecific antibodies, bispecific antigen binding fragments, and/or bispecific antibody domains) that bind to two different epitopes with at least one being an epitope of an IL1RAP polypeptide (e.g., a human IL1RAP polypeptide). In some cases, a bispecific binder provided herein can be designed to bind to two different epitopes of the same IL1RAP polypeptide (e.g., a human IL1RAP polypeptide). In some cases, a bispecific binder provided herein can bind to an IL1RAP polypeptide (e.g., a human IL1RAP polypeptide) and to an epitope on a different polypeptide (e.g., a CD3 polypeptide). Bispecific binders can be produced by chemically conjugating two different binders (e.g., antibodies, antigen binding fragments, and/or antibody domains) together. Bispecific binders also can be produced by fusing two antibody-producing cells, e.g., hybridomas, to make a hybrid cell line that produces two different heavy and two different light chains within the same cell, which can result in, for example, bispecific IgG molecules. See, Brinkmann and Kontermann, MAbs., 9(2):182-212 (2017).

In some cases, a binder (e.g., an antibody, antigen binding fragment, antibody domain, CAR, and/or cell engager) provided herein can be fused or conjugated (e.g., covalently or non-covalently attached) to another polypeptide or other moiety to provide a fusion protein or conjugate. For example, a binder (e.g., an antibody, antigen binding fragment, antibody domain, CAR, and/or cell engager) provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an enzyme, a metal chelate, a drug, a radioisotope, and/or a cytotoxic agent. Any appropriate method can be used to conjugate (e.g., covalently or non-covalently attach) another polypeptide or other moiety to a binder (e.g., an antibody, antigen binding fragment, antibody domain, CAR, and/or cell engager) provided herein. For example, another polypeptide or other moiety can be conjugated to a binder (e.g., an antibody, antigen binding fragment, antibody domain, CAR, and/or cell engager) provided herein using the methods described in U.S. Pat. No. 8,021,661.

In some cases, a binder (e.g., an antibody, antigen binding fragment, antibody domain, CAR, cell engager, and/or ADC) provided herein can be modified with a moiety that improves its stabilization and/or retention in circulation, for example, in blood, serum, or other tissues by, for example, at least 1.5-, 2-, 5-, 10-, or 50-fold. For example, a binder (e.g., an antibody, antigen binding fragment, antibody domain, CAR, cell engager, and/or ADC) provided herein can be attached (e.g., covalently or non-covalently attached) to a polymer such as a substantially non-antigenic polymer. Examples of substantially non-antigenic polymers that can be used as described herein include, without limitation, polyalkylene oxides and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a binder (e.g., an antibody, antigen binding fragment, antibody domain, CAR, cell engager, and/or ADC) provided herein can be attached (e.g., covalently or non-covalently) to a water soluble polymer. Examples of water soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, polyoxyethylenated polyols, and copolymers thereof and/or block copolymers thereof provided that the water solubility of the copolymer or block copolymers is maintained.

In some cases, a binder (e.g., an antibody, antigen binding fragment, antibody domain, CAR, cell engager, and/or ADC) provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a binder (e.g., an antibody, antigen binding fragment, antibody domain, CAR, cell engager, and/or ADC) provided herein can be covalently attached to polyoxyethylene.

Some embodiments provided herein also include ADCs. The term “ADC” as used herein refers to a conjugate that includes (a) an antigen binding domain and (b) at least one drug covalently linked directly or indirectly to that antigen binding domain. In some cases, an ADC described herein can include (a) an antigen binding domain having the ability to bind to an IL1RAP polypeptide (e.g., a human IL1RAP polypeptide) and (b) at least one drug covalently linked directly or indirectly to that antigen binding domain. Any appropriate binder (e.g., an antibody, antigen binding fragment, and/or antibody domain) provided herein and having the ability to bind to an IL1RAP polypeptide (e.g., a human IL1RAP polypeptide) can be used as an antigen binding domain to make an ADC described herein. For example, any of the binders set forth in Table 15 can be used to make an ADC having the ability to bind to an IL1RAP polypeptide (e.g., a human IL1RAP polypeptide). Examples of drugs that can be used to make an ADC described herein include, without limitation, auristatins (e.g., monomethyl auristatin E (MMAE)), mertansine (DM-1), and pyrrolobenzodiazepine (PBD) dimers. Any appropriate ADC linker can be used to covalently attach one or more drugs to an antigen binding domain having the ability to bind to an IL1RAP polypeptide (e.g., a human IL1RAP polypeptide) to form an ADC provided herein. For example, cleavable or non-cleavable ADC linkers can be used to covalently attach one or more drugs to an antigen binding domain having the ability to bind to an IL1RAP polypeptide (e.g., a human IL1RAP polypeptide) to form an ADC provided herein. Examples of ADC linkers can be used to covalently attach one or more drugs to an antigen binding domain having the ability to bind to an IL1RAP polypeptide (e.g., a human IL1RAP polypeptide) to form an ADC provided herein include, without limitation, ADC disulfide linkers, ADC hydrazone linkers, ADC peptide linkers, ADC thioether linkers, and ADC PEG-containing linkers.

Some embodiments provided herein also include nucleic acid molecules (e.g., isolated nucleic acid molecules) having a nucleic acid sequence encoding at least part of a binder (e.g., an antibody, antigen binding fragment, antibody domain, CAR, and/or cell engager) provided herein. For example, an isolated nucleic acid molecule provided herein can include a nucleic acid sequence encoding a VH domain set forth in FIG. 7A, 7B, 8A, 8B, 9A, 9B, 10A, 10B, 20, 21, 22, or 23. A nucleic acid provided herein (e.g., an isolated nucleic acid molecule) can be single stranded or double stranded nucleic acid of any appropriate type (e.g., DNA, RNA, or DNA/RNA hybrids).

Some embodiments provided herein also include vectors (e.g., plasmid vectors or viral vectors) containing one or more nucleic acids provided herein. An example of a plasmid vector that can be designed to include one or more nucleic acids having a nucleic acid sequence encoding at least part of a binder (e.g., an antibody, antigen binding fragment, antibody domain, CAR, and/or cell engager) provided herein includes, without limitation, phagemids. Examples of viral vectors that can be designed to include one or more nucleic acids having a nucleic acid sequence encoding at least part of a binder (e.g., an antibody, antigen binding fragment, antibody domain, CAR, and/or cell engager) provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a binder (e.g., an antibody, antigen binding fragment, antibody domain, CAR, and/or cell engager) provided herein.

In some cases, a vector (e.g., a plasmid vector or a viral vector) provided herein can include a nucleic acid sequence encoding scFv or antibody domain (e.g., a VH domain) provided herein. In some cases, a vector (e.g., a plasmid vector or a viral vector) provided herein can include a nucleic acid sequence encoding CAR provided herein. In some cases, a vector (e.g., a plasmid vector or a viral vector) provided herein can include a nucleic acid sequence encoding cell engager provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding at least part of a binder (e.g., an antibody, antigen binding fragment, antibody domain, CAR, and/or cell engager) provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors) and AAV promoters.

Any appropriate method can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein. For example, molecule cloning techniques can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein as described elsewhere (see, e.g., Sambrook et al., Molecular Cloning: A Laboratory Manual, 2nd edition, Cold Spring Harbor Laboratory, N Y (1989); and Ausubel et al., Current Protocols in Molecular Biology, Green Publishing Associates and John Wiley & Sons, New York, N.Y. (1994)).

Some embodiments provided herein also include host cells that include a nucleic acid provided herein (e.g., a nucleic acid having a nucleic acid sequence encoding at least part of a binder (e.g., an antibody, antigen binding fragment, antibody domain, CAR, and/or cell engager) provided herein). Host cells that can be designed to include one or more nucleic acids provided herein can be prokaryotic cells or eukaryotic cells. Examples of prokayotic cells that can be designed to include a nucleic acid provided herein include, without limitation, E. coli (e.g., Tb-1, TG-1, DH5α, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukayotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a binder provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, N Y (1989); Davis et al., Basic Methods in Molecular Biology (1986); and Neumann et al., EMBO J., 1:841 (1982)).

In some cases, cells such as T cells, stem cells (e.g., induced pluripotent stem cells or mesenchymal stem cells), or NK cells can be designed to express one or more nucleic acids encoding a CAR described herein. For example, a population of T cells can be infected with viral vectors designed to express nucleic acid encoding a CAR described herein (e.g., a CAR having the ability to bind to an IL1RAP polypeptide).

In some cases, cells such as T cells, stem cells (e.g., induced pluripotent stem cells or mesenchymal stem cells), or NK cells can be designed to express one or more nucleic acids encoding a cell engager described herein. For example, a population of T cells can be infected with viral vectors designed to express nucleic acid encoding a cell engager described herein (e.g., a cell engager having the ability to bind to an IL1RAP polypeptide).

In some cases, a binder (e.g., an antibody, antigen binding fragment, antibody domain, CAR, and/or cell engager) provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) having cancer to treat that mammal. In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) to reduce the number of cancer cells within the mammal and/or to increase the survival of the mammal suffering from cancer. For example, a binder (e.g., an antibody, antigen binding fragment, antibody domain, cell engager, and/or ADC) provided herein having the ability to bind to an IL1RAP polypeptide (e.g., a human IL1RAP polypeptide) can be formulated as a pharmaceutical composition for administration to a mammal (e.g. a human). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi, et al., Eur. J Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, a sugar (e.g., sorbitol, mannitol, sucrose, dextrose, or trehalose), or combinations thereof. For example, a pharmaceutical composition designed to include a binder (e.g., an antibody, antigen binding fragment, antibody domain, CAR, cell engager, and/or ADC) provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imanitib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, or combinations thereof. For example, a pharmaceutical composition provided herein can be formulated to include one or more binders (e.g., one or more antibodies, one or more antigen binding fragments, one or more antibody domains, one or more cells designed to express a CAR having the ability to bind to an IL1RAP polypeptide, one or more cell engagers, and/or one or more ADCs) provided herein in combination with one or more checkpoint inhibitors such as anti-PD-1 antibodies or PD-1 inhibitors (e.g., cemiplimab, nivolumab, pembrolizumab, JTX-4014, spartalizumab, camrelizumab, sintilimab, tislelizumab, toripalimab, dostarlimab, INCMGA00012, AMP-224, or AMP-514), anti-PD-L1 antibodies or PD-L1 inhibitors (e.g., avelumab, durvalumab, atezolizumab, KN035, CK-301, AUNP12, CA-170, or BMS-986189), and/or anti-CTLA-4 antibodies (e.g., ipilimumab).

In some cases, when a pharmaceutical composition is formulated to include one or more binders (e.g., one or more antibodies, one or more antigen binding fragments, one or more antibody domains, one or more cells designed to express a CAR having the ability to bind to an IL1RAP polypeptide, one or more cell engagers, and/or one or more ADCs) provided herein, any appropriate concentration of the binder can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, CAR⁺ cell population, cell engager, and/or ADC) provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, cell engager, and/or ADC) provided herein. In some cases, a pharmaceutical composition containing a binder (e.g., an antibody, antigen binding fragment, and/or antibody domain) provided herein can be formulated as a dosage form with a titer of the binder being from about 1×10⁵ to about 1×10¹² (e.g., from about 1×10⁵ to about 1×10¹⁰, from about 1×10⁵ to about 1×10⁸, from about 1×10⁶ to about 1×10¹², from about 1×10⁶ to about 1×10¹², from about 1×10⁸ to about 1×10¹², from about 1×10⁹ to about 1×10¹², from about 1×10⁶ to about 1×10¹¹, or from about 1×10⁷ to about 1×10¹⁰).

In some cases, when a pharmaceutical composition is formulated to include one or more nucleic acids (e.g., vectors such as viral vectors) encoding at least part of a binder (e.g., an antibody, antigen binding fragment, antibody domain, CAR, and/or cell engager) provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

In some cases, a pharmaceutical composition designed to include a binder (e.g., an antibody, antigen binding fragment, antibody domain, cell engager, and/or ADC) provided herein can be formulated to include one or more agents capable of reducing aggregation of the binder when formulated. Examples of such agents that can be used as described herein include, without limitation, methionine, arginine, lysine, aspartic acid, glycine, glutamic acid, and combinations thereof. In some cases, one or more of these amino acids can be included within the formulation at a concentration from about 0.5 mM to about 145 mM (e.g., from about 1 mM to about 145 mM, from about 10 mM to about 145 mM, from about 100 mM to about 145 mM, from about 0.5 mM to about 125 mM, from about 0.5 mM to about 100 mM, from about 0.5 mM to about 75 mM, or from about 10 mM to about 100 mM).

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more binders (e.g., one or more antibodies, one or more antigen binding fragments, one or more antibody domains, one or more cell engagers, and/or one or more ADCs) provided herein can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

Some embodiments provided herein also include methods for administering a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) to a mammal (e.g., a human). For example, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g., a human) having cancer to treat that mammal. In some cases, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g. a human) to reduce the number of cancer cells within the mammal and/or to increase the survival of the mammal suffering from cancer.

Any appropriate cancer can be treated using a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein). For example, a mammal (e.g., a human) having cancer can be treated by administering a composition (e.g., a pharmaceutical composition) containing one or more binders (e.g., one or more antibodies, one or more antigen binding fragments, one or more antibody domains, one or more cell engagers, and/or one or more ADCs) provided herein to that mammal. Examples of cancers that can be treated as described herein include, without limitation, Ewing's sarcoma, acute myeloid leukemia (AIL), liver cancer, colorectal cancer, brain cancer, skin cancer (e.g., melanoma), lung cancer, prostate cancer, breast cancer (e.g., PR positive breast cancer, ER positive breast cancer, HER2 positive breast cancer, or triple negative breast cancer), ovarian cancer, cervical cancer, esophageal cancer, glioma, kidney cancer, mesothelioma, and pancreatic cancer. In some cases, a solid cancer such as liver cancer, colorectal cancer, brain cancer, skin cancer (e.g., melanoma), lung cancer, prostate cancer, breast cancer (e.g., PR positive breast cancer, ER positive breast cancer, HER2 positive breast cancer, or triple negative breast cancer), ovarian cancer, cervical cancer, esophageal cancer, glioma, kidney cancer, mesothelioma, or pancreatic cancer can be treated as described herein. In some cases, cancer such as lymphoma (e.g., B cell lymphomas such as diffuse large cell lymphoma (DLBCL)), leukemia (e.g., chronic lymphocytic leukemia (CLL) and acute lymphoblastic leukemia (ALL)), or acute myeloid leukemia can be treated as described herein. In some cases, a mammal (e.g., a human) having an IL1RAP⁺ cancer (e.g., an IL1RAP⁺ Ewing's sarcoma, an IL1RAP⁺ AML, an IL1RAP⁺ liver cancer, IL1RAP⁺ colorectal cancer, IL1RAP⁺ brain cancer, IL1RAP⁺ skin cancer (e.g., IL1RAP⁺ melanoma), IL1RAP⁺ lung cancer, IL1RAP⁺ prostate cancer, IL1RAP⁺ breast cancer (e.g., IL1RAP⁺ PR positive breast cancer, IL1RAP⁺ ER positive breast cancer, IL1RAP HER2 positive breast cancer, or IL1RAP⁺ triple negative breast cancer), IL1RAP⁺ ovarian cancer, IL1RAP⁺ cervical cancer, IL1RAP⁺ esophageal cancer, IL1RAP⁺ glioma, IL1RAP⁺ kidney cancer, IL1RAP⁺ mesothelioma, or IL1RAP⁺ pancreatic cancer) can be administered a composition (e.g., a pharmaceutical composition) containing one or more binders (e.g., one or more antibodies, one or more antigen binding fragments, one or more antibody domains, one or more cell engagers, and/or one or more ADCs) provided herein to treat that mammal (e.g., to reduce the number of cancer cells within the mammal).

Any appropriate method can be used to administer a composition (e.g., a pharmaceutical composition) provided herein to a mammal (e.g., a human). For example, a composition provided herein (e.g., a pharmaceutical composition containing one or more binders provided herein such as one or more antibodies, one or more antigen binding fragments, one or more antibody domains, one or more cell engagers, and/or one or more ADCs provided herein) can be administered to a mammal (e.g., a human) intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that reduces the number of cancer cells within a mammal having cancer without producing significant toxicity to the mammal. In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that increases the survival time of a mammal having cancer as compared to a control mammal having comparable cancer and not treated with the composition. For example, an effective amount of a binder (e.g., an antibody, antigen binding fragment, antibody domain, cell engager, and/or ADC) provided herein can be from about 0.001 mg/kg to about 100 mg/kg (e.g., from about 0.001 mg/kg to about 90 mg/kg, from about 0.001 mg/kg to about 80 mg/kg, from about 0.001 mg/kg to about 70 mg/kg, from about 0.001 mg/kg to about 60 mg/kg, from about 0.001 mg/kg to about 50 mg/kg, from about 0.001 mg/kg to about 40 mg/kg, from about 0.001 mg/kg to about 30 mg/kg, from about 0.005 mg/kg to about 100 mg/kg, from about 0.01 mg/kg to about 100 mg/kg, from about 0.05 mg/kg to about 100 mg/kg, from about 0.1 mg/kg to about 100 mg/kg, from about 0.5 mg/kg to about 100 mg/kg, from about 1 mg/kg to about 100 mg/kg, from about 5 mg/kg to about 100 mg/kg, from about 0.01 mg/kg to about 25 mg/kg, from about 0.1 mg/kg to about 30 mg/kg, from about 0.15 mg/kg to about 25 mg/kg, from about 0.2 mg/kg to about 20 mg/kg, from about 0.5 mg/kg to about 20 mg/kg, from about 1 mg/kg to about 30 mg/kg, from about 1 mg/kg to about 25 mg/kg, from about 1 mg/kg to about 20 mg/kg, from about 2 mg/kg to about 20 mg/kg, from about 5 mg/kg to about 30 mg/kg, from about 10 mg/kg to about 30 mg/kg, from about 15 mg/kg to about 30 mg/kg, from about 20 mg/kg to about 30 mg/kg, from about 3 mg/kg to about 30 mg/kg, from about 0.5 mg/kg to about 10 mg/kg, from about 1 mg/kg to about 10 mg/kg, from about 1 mg/kg to about 5 mg/kg, or from about 1 mg/kg to about 3 mg/kg). The effective amount can remain constant or can be adjusted as a sliding scale or variable dose depending on the mammal's response to treatment. Various factors can influence the actual effective amount used for a particular application. For example, the severity of cancer when treating a mammal having cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective amount of a composition provided herein (e.g., a pharmaceutical composition containing one or more binders provided herein) that is administered.

In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that reduces the number of cancer cells within a mammal having cancer without producing significant toxicity to the mammal. In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that increases the survival time of a mammal having cancer as compared to a control mammal having comparable cancer and not treated with the composition. For example, an effective frequency of administration of a pharmaceutical composition provided herein such as a pharmaceutical composition containing one or more binders provided herein can be from about twice daily to about once a year (e.g., from about twice daily to about once a month, from about twice daily to about once a week, from about once daily to about once a month, or from one once daily to about once a week). In some cases, the frequency of administration of a pharmaceutical composition provided herein such as a pharmaceutical composition containing one or more binders provided herein can be daily. The frequency of administration of a pharmaceutical composition provided herein such as a pharmaceutical composition containing one or more binders provided herein can remain constant or can be variable during the duration of treatment. Various factors can influence the actual effective frequency used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective frequency of administration of a composition provided herein (e.g., a pharmaceutical composition containing one or more binders provided herein).

In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that reduces the number of cancer cells within a mammal without producing significant toxicity to the mammal. In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that increases the survival time of a mammal having cancer as compared to a control mammal having comparable cancer and not treated with the composition. For example, an effective duration of administration of a pharmaceutical composition provided herein such as a pharmaceutical composition containing one or more binders provided herein can vary from a single time point of administration to several weeks to several months (e.g., 4 to 12 weeks). Multiple factors can influence the actual effective duration used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective duration of administration of a composition provided herein (e.g., a pharmaceutical composition containing one or more binders provided herein).

In some cases, a binder (e.g., an antibody, antigen binding fragment, and/or antibody domain) provided herein can be used to detect the presence or absence of an IL1RAP polypeptide (e.g., a human IL1RAP polypeptide) in vitro, in situ, or in vivo (e.g., in vivo imaging within a mammal such as a human). For example, a binder (e.g., an antibody, antigen binding fragment, and/or antibody domain) provided herein can be designed to include a label (e.g., a covalently attached radioactive, enzymatic, colorimetric, or fluorescent label). The labelled binder can be used to detect the presence or absence of an IL1RAP polypeptide (e.g., a human IL1RAP polypeptide) within a biological sample in vitro. Examples of biological samples that can be assessed using a binder (e.g., an antibody, antigen binding fragment, and/or antibody domain) provided herein include, without limitation, serum samples, plasma samples, tissue samples, biopsy samples, cell line samples, and tissue culture samples. In some cases, a biological sample that can be assessed as described herein can include mammalian body tissues and/or cells such as leukocytes, ovary tissue or cells, prostate tissue or cells, heart tissue or cells, placenta tissue or cells, pancreas tissue or cells, liver tissue or cells, spleen tissue or cells, lung tissue or cells, breast tissue or cells, head and neck tissue or cells, endometrium tissue or cells, colon tissue or cells, colorectal tissue or cells, cervix tissue or cells, stomach tissue or cells, or umbilical tissue or cells that may express an IL1RAP polypeptide (e.g., a human IL1RAP polypeptide). In some cases, a binder (e.g., an antibody, antigen binding fragment, and/or antibody domain) provided herein can be immobilized, e.g., on a support, and retention of an IL1RAP polypeptide (e.g., a human IL1RAP polypeptide) from a biological sample on the support can be detected, and/or vice versa. In some cases, a binder (e.g., an antibody, antigen binding fragment, and/or antibody domain) provided herein can be used in applications such as fluorescence polarization, microscopy, ELISA, centrifugation, chromatography, and/or cell sorting (e.g., fluorescence activated cell sorting).

In some cases, a binder (e.g., an antibody, antigen binding fragment, and/or antibody domain) provided herein containing a label (e.g., a covalently attached radioactive label) can be used to detect the presence or absence of an IL1RAP polypeptide (e.g., a human IL1RAP polypeptide) within a mammal (e.g., a human). For example, a binder (e.g., an antibody, antigen binding fragment, and/or antibody domain) provided herein that is labelled (e.g., covalently labelled) with a radiolabel or an MRI detectable label can be administered to a mammal (e.g., a human), and that mammal can be assessed using a means for detecting the detectable label. In some cases, a mammal can be scanned to evaluate the location(s) of a labelled binder provided herein within the mammal. For example, the mammal can be imaged using NMR or other tomographic techniques.

Examples of labels that can be attached (e.g., covalently or non-covalently attached) to a binder (e.g., an antibody, antigen binding fragment, and/or antibody domain) provided herein include, without limitation, radiolabels such as ¹³¹I, ¹¹¹In, ¹²³I, ^{99m}Tc, ³²P, ³³P, ¹²⁵I, ³H ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as a peroxidase or a phosphatase. In some cases, short-range radiation emitters such as isotopes detectable by short-range detector probes can be used.

Some embodiments of the methods and compositions provided herein include chimeric antigen receptors (CARs), which specifically bind to interleukin-1 receptor accessory protein (IL1RAP). Some embodiments include nucleic acids encoding such CARs, and cells containing such CARs. Some embodiments include the use of such CARs in safe and effective therapies for a cancer, such as an IL1RAP-expressing cancer, such as a Ewing's sarcoma.

IL1RAP has restricted expression on normal proteins. IL1RAP is expressed on the cell surface in some cancers including Ewing's sarcoma and AML. Thus, IL1RAP is a therapeutic target for certain cancer cells. A series of IL1RAP-binding moieties were identified through screening methods. The IL1RAP-binding moieties were expressed and tested in an antibody-based format. Some of these binders were found to function in the context of a CAR. CAR-modified T cells expressing the moieties recognized and lysed cancer cells from a Ewing's sarcoma cell line. Thus, biochemical and functional studies have identified a new anti-cancer reagent, an IL1RAP-specific CARs, disclosed herein as RJ104 and RJ107.

Some embodiments of the methods and compositions provided herein include aspects disclosed in: Agerstam H, et al., (2015) Proc Natl Acad Sci, USA, 112:10786-10791; Awada A, et al., (2018) Annal Oncol 29, suppl 8, pg viii418; Warda W, et al., (2018) Cancer Res, 79:663-675; and Haso W, et al., (2013) Blood, 121:1165-1174, which are each expressly incorporated by reference in its entirety.

### Definitions

Unless defined otherwise, all technical and scientific terms used herein have the same meaning as commonly understood by one of ordinary skill in the art to which the invention pertains.

As used herein, “a” or “an” may mean one or more than one.

“About” as used herein when referring to a measurable value is meant to encompass variations of ±20% or ±10%, more preferably ±5%, even more preferably ±1%, and still more preferably ±0.1% from the specified value.

As used herein, “nucleic acid” or “nucleic acid molecule” have their plain and ordinary meaning in view of the whole specification and may to refer to, for example, polynucleotides, such as deoxyribonucleic acid (DNA) or ribonucleic acid (RNA), oligonucleotides, fragments generated by the polymerase chain reaction (PCR), or fragments generated by any of ligation, scission, endonuclease action, or exonuclease action. Nucleic acid molecules can be composed of monomers that are naturally-occurring nucleotides (such as DNA or RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, or azido groups, or sugars can be functionalized as ethers or esters. Moreover, the entire sugar moiety can be replaced with sterically and electronically similar structures, such as aza-sugars or carbocyclic sugar analogs. Examples of modifications in abase moiety include alkylated purines or pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded. In some embodiments, a nucleic acid sequence encoding a fusion protein is provided. In some embodiments, the nucleic acid encoding the chimeric antigen receptor specific for IL1RAP is RNA or DNA.

As used herein, “coding for” or “encoding” has its plain and ordinary meaning when read in light of the specification, and includes, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

As used herein, “chimeric antigen receptor” has its plain and ordinary meaning when read in light of the specification, and may include but is not limited to, for example, a synthetically designed receptor comprising a ligand binding domain of an antibody or other protein sequence that binds to a molecule associated with a disease or disorder and is, preferably, linked via a spacer domain to one or more intracellular signaling domains of a cell, such as a T cell, or other receptors, such as one or more costimulatory domains. Chimeric receptor can also be referred to as artificial cell receptors or T cell receptors, chimeric cell receptors or T cell receptors, chimeric immunoreceptors, or CARs. These receptors can be used to graft the specificity of a monoclonal antibody or binding fragment thereof onto a cell, preferably a T-cell, with transfer of their coding sequence facilitated by viral vectors, such as a retroviral vector or a lentiviral vector. CARs can be, in some instances, genetically engineered T cell receptors designed to redirect T cells to target cells that express specific cell-surface antigens. T cells can be removed from a subject and modified so that they can express receptors that can be specific for an antigen by a process called adoptive cell transfer. The T cells are reintroduced into the patient where they can then recognize and target an antigen. CARs are also engineered receptors that can graft an arbitrary specificity onto an immune receptor cell. CARs are considered by some investigators to include the antibody or antibody fragment, preferably an antigen binding fragment of an antibody, the spacer, signaling domain, and transmembrane region. Due to the surprising effects of modifying the different components or domains of the CAR described herein, such as the epitope binding region (for example, antibody fragment, scFv, or portion thereof), spacer, transmembrane domain, and/or signaling domain), the components of the CAR are frequently distinguished throughout this disclosure in terms of independent elements. The variation of the different elements of the CAR can, for example, lead to stronger binding affinity for a specific epitope or antigen.

The CARs graft the specificity of a monoclonal antibody or binding fragment thereof or scFv onto a T cell, with the transfer of their coding sequence facilitated by vectors. In order to use CARs as a therapy for a subject in need, a technique called adoptive cell transfer is used in which T cells are removed from a subject and modified so that they can express the CARs that are specific for an antigen. The T cells, which can then recognize and target an antigen, are reintroduced into the patient.

As used herein, an “antibody” has its plain and ordinary meaning when read in light of the specification, and includes, for example, a large Y-shape protein produced by plasma cells that is used by the immune system to identify and neutralize foreign objects such as bacteria and viruses. The antibody protein can comprise four polypeptide chains; two identical heavy chains and two identical light chains connected by disulfide bonds. Each chain is composed of structural domains called immunoglobulin domains. These domains can contain about 70, 80, 90, 100, 110, 120, 130, 140, or 150 amino acids or any number of amino acids in between in a range defined by any two of these values and are classified into different categories according to their size and function. In some embodiments, the ligand binding domain comprises an antibody or binding fragment thereof or scFv, a receptor ligand or mutants thereof, peptide, and/or polypeptide affinity molecule or binding partner. In some embodiments, the ligand binding domain is an antibody fragment, desirably, a binding portion thereof. In some embodiments, the antibody fragment or binding portion thereof present on a CAR is specific for a ligand on a B-cell. In some embodiments, the antibody fragment or binding portion thereof present on a CAR or TcR is specific for a ligand. In some embodiments, the antibody fragment or binding portion thereof present on a CAR is specific for IL1RAP. In some embodiments, the ligand binding domain is an antibody fragment or a binding portion thereof, such as a single chain variable fragment (scFv). In some embodiments, the antibody fragment or binding portion thereof present on a CAR comprises one or more domains from a humanized antibody, or binding portion thereof.

As used herein, a “single chain variable fragment” or “scFv” has its plain and ordinary meaning when read in light of the specification, and includes, for example, a fusion protein of the variable regions of the heavy (VH) and light chains (VL) of immunoglobulins, connected with a short linker peptide of ten to 25 amino acids or about 25 amino acids. In some embodiments, a CAR is provided, wherein the CAR comprises a scFv specific for IL1RAP.

The strength of binding of a ligand is referred to as the binding affinity and can be determined by direct interactions and solvent effects. A ligand can be bound by a “ligand binding domain.” A ligand binding domain, for example, can refer to a conserved sequence in a structure that can bind a specific ligand or a specific epitope on a protein. The ligand binding domain or ligand binding portion can comprise an antibody or binding fragment thereof or scFv, a receptor ligand or mutants thereof, peptide, and/or polypeptide affinity molecule or binding partner. Without being limiting, a ligand binding domain can be a specific protein domain or an epitope on a protein that is specific for a ligand or ligands.

Some embodiments include a spacer. In some embodiments, the peptide spacer is 15 amino acids or less but not less than 1 or 2 amino acids. In some embodiments, the spacer is a polypeptide chain. In some aspects, the polypeptide chain may range in length, such as from 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101,102, 103, 104, 105, 106,107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239 or 240 amino acids or a length within a range defined by any two of the aforementioned lengths. A spacer can comprise any 20 amino acids, for example, in any order to create a desirable length of polypeptide chain in a CAR, which includes the amino acids arginine, histidine, lysine, aspartic acid, glutamic acid, serine, threonine, asparagine, glutamine, cysteine, glycine, proline, alanine, valine, isoleucine, methionine, phenylalanine, tyrosine or tryptophan. A spacer sequence can be a linker between the scFv (or ligand binding domain) and the transmembrane domain of the CAR. In some embodiments, the chimeric antigen receptor further comprises a sequence encoding a spacer. In some embodiments, the spacer comprises a sequence with a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132,133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239 or 240 amino acids or a length within a range defined by any two of the aforementioned lengths. In some embodiments, the spacer resides between the scFv and the transmembrane region of the CAR. In some embodiments, the spacer resides between the ligand binding domain of the CAR and the transmembrane region of the CAR.

A spacer may also be customized, selected, or optimized for a desired length so as to improve or modulate binding of scFv domain to the target cell, which may increase cytotoxic efficacy. In some embodiments, the linker or spacer between the scFv domain or ligand binding domain and the transmembrane can be 25 to 55 amino acids in length (e.g., at least, equal to 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids or a length within a range defined by any two of the aforementioned lengths).

In some embodiments, the spacer comprises a CD8 hinge. In some embodiments, the spacer comprises a hinge region of a human antibody. In some embodiments, the spacer comprises an IgG4 hinge. In some embodiments, the IgG4 hinge region is a modified IgG4 hinge. A “modified IgG4 hinge” as described herein can refer to a hinge region that can have at least 90%, 91%, 92%, 93%, 94% 95%, 96%, 97%, 98%, 99%, or 100% sequence identity or a sequence identity within a range defined by any two of the aforementioned percentages, with a hinge region amino acid sequence as set forth in a spacer, such as a short spacer listed in TABLE 2. In some embodiments, the CAR comprises an S spacer, M spacer or an L spacer. Example sequences are listed in TABLE 2.

As used herein, a “de-immunized spacer” has its plain and ordinary meaning when read in light of the specification, and includes, for example, a spacer that induces little to no immune response or a diminished or reduced immune response from a patient. In some embodiments, the CAR comprises a spacer, wherein the spacer does not induce an immune response in a subject, such as a human. It is important that the spacer does not induce an immune response or induces a reduced or diminished or low immune response in a subject, such as a human, in order to prevent or reduce the ability of the immune system to attack the chimeric antigen receptor.

In some embodiments, the transmembrane domain is a region of a membrane-spanning protein that is hydrophobic that can reside in the bilayer of a cell to anchor a protein that is embedded to the biological membrane. Without being limiting, the topology of the transmembrane domain can be a transmembrane alpha helix. In some embodiments, a CAR comprises a transmembrane domain. In some embodiments, the transmembrane domain comprises a CD8 transmembrane sequence or a fragment thereof or a CD28 transmembrane sequence or a fragment thereof that is a length of 10, 11, 12, 13, 14, 15, 16, 17, 18, 19, 20, 21, 22, 23, 24, 25, 26, 27, or 28 amino acids or a length within a range defined by any two of the aforementioned lengths. In some embodiments, the CD8 transmembrane sequence or a fragment thereof or the CD28 transmembrane sequence or fragment thereof comprises 28 amino acids in length.

In some embodiments, the signaling domains, such as primary signaling domains or costimulatory domains, include an intracellular or cytoplasmic domain of a protein or a receptor protein that interacts with components within the interior of the cells and is capable of or configured to relay or participate in the relaying of a signal. Such interactions in some aspects can occur through the intracellular domain communicating via specific protein-protein or protein-ligand interactions with an effector molecule or an effector protein, which in turn can send the signal along a signal chain to its destination. In some embodiments, the signaling domain includes one or more co-stimulatory domains. In some embodiments, the one or more costimulatory domains include a signaling moiety that provides a T-cell with a signal, which, in addition to the primary signal provided by for instance the CD3 zeta chain of the TCR/CD3 complex, enhances a response such as a T-cell effector response, such as, for example, an immune response, activation, proliferation, differentiation, cytokine secretion, cytolytic activity, perforin or granzyme activity or any combination thereof. In some embodiments, the intracellular signaling domain or the co-stimulatory domain can include all or a portion of CD27, CD28, 4-1BB, OX40, CD30, CD40, ICOS, lymphocyte function-associated antigen-1 (LFA-1), CD2, CD7, LIGHT, NKG2C, or B7-H3, or a ligand that specifically binds with CD83 or any combination thereof.

As used herein, a “ribosome skip sequence” has its plain and ordinary meaning when read in light of the specification, and includes, for example, a sequence that during translation, forces the ribosome to “skip” the ribosome skip sequence and translate the region after the ribosome skip sequence without formation of a peptide bond. Several viruses, for example, have ribosome skip sequences that allow sequential translation of several proteins on a single nucleic acid without having the proteins linked via a peptide bond. As described herein, this is the “linker” sequence. In some embodiments of the nucleic acids provided herein, the nucleic acids comprise a ribosome skip sequence between the sequence for the chimeric antigen receptor and the sequence of the marker protein, such that the proteins are co-expressed and not linked by a peptide bond. In some embodiments, the ribosome skip sequence is a P2A, T2A, E2A or F2A sequence.

As used herein, a “marker sequence,” has its plain and ordinary meaning when read in light of the specification, and includes, for example, a protein that is used for selecting or tracking a protein or cell that has a protein of interest. In the embodiments described herein, the fusion protein provided can comprise a marker sequence that can be selected in experiments, such as flow cytometry. In some embodiments, the marker is a truncated EGFR polypeptide (EGFRt), or a truncated HER2 polypeptide (HER2t).

As used herein, “signal sequence” for secretion, can also be referred to as a “signal peptide.” The signal peptide can be used for secretion efficiency and in some systems it is recognized by a signal recognition particle, which halts translation and directs the signal sequence to a SRP receptor for secretion. In some embodiments of the CARs provided herein, the CARs further comprise a signal sequence. In some embodiments, of the nucleic acid encoding a CAR, the nucleic acid comprises a sequence encoding a signal sequence. In some embodiments, the signal sequence is for targeting a protein to a cell membrane following translation of the protein.

As used herein, “vector” or “construct” has its plain and ordinary meaning when read in light of the specification, and includes, for example, a nucleic acid used to introduce heterologous nucleic acids into a cell that has regulatory elements to provide expression of the heterologous nucleic acids in the cell. Vectors include but are not limited to plasmid, minicircles, yeast, viral genomes, lentiviral vector, foamy viral vector, retroviral vector or gammaretroviral vector. The vector may be DNA or RNA, such as mRNA.

As used herein, “T-cells” or “T lymphocytes” can be from any mammal, preferably a primate, including monkeys or humans, a companion animal such as a dog, cat, or horse, or a domestic animal, such as a sheep, goat, or cattle. In some embodiments, the T-cells are allogeneic (from the same species but different donor) as the recipient subject; in some embodiments, the T-cells are autologous (the donor and the recipient are the same); in some embodiments, the T-cells are syngeneic (the donor and the recipients are different but are identical twins).

As used herein, “T cell precursors” refer to lymphoid precursor cells that can migrate to the thymus and become T cell precursors, which do not express a T cell receptor. All T cells originate from hematopoietic stem cells in the bone marrow. Hematopoietic progenitors (lymphoid progenitor cells) from hematopoietic stem cells populate the thymus and expand by cell division to generate a large population of immature thymocytes. The earliest thymocytes express neither CD4 nor CD8 and are therefore classed as double-negative (CD4-CD8−) cells. As they progress through their development, they become double-positive thymocytes (CD4+CD8+), and finally mature to single-positive (CD4+CD8− or CD4-CD8+) thymocytes that are then released from the thymus to peripheral tissues.

As used herein, “hematopoietic stem cells” or “HSCs” are precursor cells that can give rise to myeloid cells such as, for example, macrophages, monocytes, macrophages, neutrophils, basophils, eosinophils, erythrocytes, megakaryocytes/platelets, dendritic cells and/or lymphoid lineages (such as, for example, T-cells, B-cells, or NK-cells). HSCs have a heterogeneous population in which three classes of stem cells exist, which are distinguished by their ratio of lymphoid to myeloid progeny in the blood (L/M).

As used herein, “CD4+ expressing T-cell,” or “CD4+ T-cell,” are used synonymously throughout, is also known as T helper cells, which play an important role in the immune system, and in the adaptive immune system. CD4+ T-cells also help the activity of other immune cells by releasing T-cell cytokines. These cells help, suppress or regulate immune responses. They are essential in B cell antibody class switching, in the activation and growth of cytotoxic T-cells, and in maximizing bactericidal activity of phagocytes, such as macrophages. CD4+ expressing T-cells have the ability to make some cytokines, however the amounts of cytokines made by CD4+ T-cells are not at a concentration that promotes, improves, contributes to, or induces engraftment fitness. As described herein, “CD4+ T-cells” are mature T helper-cells that play a role in the adaptive immune system.

As used herein, “CD8+ expressing T-cell” or “CD8+ T-cell,” are used synonymously throughout, is also known as a TC, cytotoxic T lymphocyte, CTL, T-killer cell, cytolytic T-cell or killer T-cell. As described herein, CD8+ T-cells are T-lymphocytes that can kill cancer cells, virally infected cells, or damaged cells. CD8+ T-cells express T-cell receptors (TCRs) that can recognize a specific antigen. CD8+ T-cells express CD8 on the surface. CD8+ expressing T-cells have the ability to make some cytokines, however the amounts of cytokines made by CD8+ T-cells are not at a concentration that promotes, improves, contributes to, or induces engraftment fitness. “CD8 T-cells” or “killer T-cells” are T-lymphocytes that can kill cancer cells, cells that are infected with viruses or cells that are damaged.

Mature T cells express the surface protein CD4 and are referred to as CD4+ T-cells. CD4+ T-cells are generally treated as having a pre-defined role as helper T-cells within the immune system. For example, when an antigen-presenting cell expresses an antigen on MIC class II, a CD4+ cell will aid those cells through a combination of cell to cell interactions (e.g. CD40 and CD40L) and through cytokines. Nevertheless, there are rare exceptions; for example, sub-groups of regulatory T-cells, natural killer cells, and cytotoxic T-cells express CD4. All of the latter CD4+ expressing T-cell groups are not considered T helper cells.

As used herein, “central memory” T-cell (or “TCM”) refers to an antigen experienced CTL that expresses CD62L or CCR-7 and CD45RO on the surface thereof, and does not express or has decreased expression of CD45RA as compared to naïve cells. In some embodiments, central memory cells are positive for expression of CD62L, CCR7, CD28, CD127, CD45RO, and/or CD95, and have decreased expression of CD54RA, as compared to naïve cells.

As used herein, “effector memory” T-cell (or “TEM”) refers to an antigen experienced T-cell that does not express or has decreased expression of CD62L on the surface thereof as compared to central memory cells, and does not express or has decreased expression of CD45RA as compared to naïve cell. In some embodiments, effector memory cells are negative for expression of CD62L and/or CCR7, as compared to naïve cells or central memory cells, and have variable expression of CD28 and/or CD45RA.

As used herein, “naïve” T-cells refers to a non-antigen experienced T lymphocyte that expresses CD62L and/or CD45RA, and/or does not express CD45RO− as compared to central or effector memory cells. In some embodiments, naïve CD8+ T lymphocytes are characterized by the expression of phenotypic markers of naïve T-cells including CD62L, CCR7, CD28, CD127, or CD45RA.

As used herein, “effector” “TE” T-cells refers to an antigen experienced cytotoxic T lymphocyte cells that do not express or have decreased expression of CD62L, CCR7, CD28, and are positive for granzyme B or perforin or both, as compared to central memory or naïve T-cells.

As used herein, “cytokines” has its plain and ordinary meaning when read in light of the specification, and includes, for example, small proteins (5-25 kDa) that are important in cell signaling. Cytokines are released by cells and affect the behavior of other cells, and sometimes the releasing cell itself, such as a T-cell. Cytokines can include, for example, chemokines, interferons, interleukins, lymphokines, or tumor necrosis factor or any combination thereof. Cytokines can be produced by a broad range of cells, which can include, for example, immune cells like macrophages, B lymphocytes, T lymphocytes, mast cells, as well as, endothelial cells, fibroblasts, or various stromal cells.

Cytokines can act through receptors and are important in the immune system as the cytokines can modulate the balance between humoral and cell-based immune responses, and they can regulate the maturation, growth, and responsiveness of particular cell populations. Some cytokines enhance or inhibit the action of other cytokines in complex ways. Without being limiting, cytokines can include, for example, Acylation stimulating protein, Adipokine, Albinterferon, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL5, CCL6, CCL7, CCL8, CCL9, Chemokine, Colony-stimulating factor, CX3CL1, CX3CR1, CXCL1, CXCL10, CXCL11, CXCL13, CXCL14, CXCL15, CXCL16, CXCL17, CXCL2, CXCL3, CXCL5, CXCL6, CXCL7, CXCL9, Erythropoietin, Gc-MAF, Granulocyte colony-stimulating factor, Granulocyte macrophage colony-stimulating factor, Hepatocyte growth factor, IL 10 family of cytokines, IL 17 family of cytokines, IL1A, IL1B, Inflammasome, Interferome, Interferon, Interferon beta 1a, Interferon beta 1b, Interferon gamma, Interferon type I, Interferon type II, Interferon type III, Interleukin, Interleukin 1 family, Interleukin 1 receptor antagonist, Interleukin 10, Interleukin 12, Interleukin 12 subunit beta, Interleukin 13, Interleukin 15, Interleukin 16, Interleukin 2, Interleukin 23, Interleukin 23 subunit alpha, Interleukin 34, Interleukin 35, Interleukin 6, Interleukin 7, Interleukin 8, Interleukin 36, Leukemia inhibitory factor, Leukocyte-promoting factor, Lymphokine, Lymphotoxin, Lymphotoxin alpha, Lymphotoxin beta, Macrophage colony-stimulating factor, Macrophage inflammatory protein, Macrophage-activating factor, Monokine, Myokine, Myonectin, Nicotinamide phosphoribosyltransferase, Oncostatin M, Oprelvekin, Platelet factor 4, Proinflammatory cytokine, Promegapoietin, RANKL, Stromal cell-derived factor 1, Talimogene laherparepvec, Tumor necrosis factor alpha, Tumor necrosis factors, XCL1, XCL2, GM-CSF, or XCR1 or any combination thereof. In some embodiments of the methods of making genetically modified T-cells, a transduced population of CD8+ expressing T-cells and/or CD4+ expressing T-cells is contacted with at least one cytokine so as to generate a transduced, cytokine-stimulated population of CD8+ T-cells and/or CD4+ T-cells. In some embodiments, the at least one cytokine utilized comprises GM-CSF, IL-7, IL-12, IL-15, IL-18, IL-2 or IL-21 or any combination thereof. In some embodiments, the period of contact with the cytokine is at least one day, such as for at least 1, 2, 3, 4, 5, 6, 7, 8, 9, 10, 11, 12, 13, 14, 15, 16, 17, 18, 19, or 20 days or any time that is within a range of times defined by any two of the aforementioned time points.

As used herein, “interleukins” or IL are cytokines that the immune system depends largely upon. Examples of interleukins, which can be utilized herein, for example, include IL-1, IL-2, IL-3, IL-4, IL-5, IL-6, Il-7, IL-8/CXCL8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, or IL-36 or any combination thereof. Contacting T-cells with interleukins can have effects that promote, support, induce, or improve engraftment fitness of the cells. IL-1, for example can function in the maturation and proliferation of T-cells. IL-2, for example, can stimulate growth and differentiation of T-cell response. IL-3, for example, can promote differentiation and proliferation of myeloid progenitor cells. IL-4, for example, can promote proliferation and differentiation. IL-7, for example, can promote differentiation and proliferation of lymphoid progenitor cells, involved in B, T, and NK cell survival, development, and homeostasis. IL-15, for example, can induce production of natural killer cells. IL-21, for example, co-stimulates activation and proliferation of CD8+ T-cells, augments NK cytotoxicity, augments CD40-driven B cell proliferation, differentiation and isotype switching, and promotes differentiation of Th17 cells.

As used herein, “propagating cells” or propagation refers to steps to allow proliferation, expansion, growth and reproduction of cells. For example, cultures of CD8+ T-cells and CD4+ T-cells can typically be incubated under conditions that are suitable for the growth and proliferation of T lymphocytes. In some embodiments of the method of making genetically modified T-cells, which have a chimeric antigen receptor, the CD4+ expressing T-cells are propagated for at least 1 day and may be propagated for 20 days, such as 1, 2, 3, 4, 5, 6, 7, 8, 9, 10, 11, 12, 13, 14, 15, 16, 17, 18, 19, or 20 days or for a period that is within a range defined by any two of the aforementioned time periods. In some embodiments of the method of making genetically modified T-cells, which have a chimeric antigen receptor, the CD8+ expressing T-cells are propagated for at least 1 day and may be propagated for 20 days, such as 1, 2, 3, 4, 5, 6, 7, 8, 9, 10, 11, 12, 13, 14, 15, 16, 17, 18, 19, or 20 days or for a period that is within a range defined by any two of the aforementioned time periods.

In another alternative, the expansion method or propagation can further comprise adding anti-CD3 and/or anti CD28 antibody to the culture medium (e.g., at a concentration of at least 0.5 ng/ml). In another alternative, the method of making genetically modified T-cells, which have a chimeric antigen receptor method can further comprise adding IL-2, IL-15, or IL-21 or any combination thereof to the culture medium (e.g., wherein the concentration of IL-2 is at least 10 units/mL). In another alternative, the method of making genetically modified T-cells, which have a chimeric antigen receptor method can further comprise adding IL-7, IL-15, or IL21 or any combination thereof to the culture medium (e.g., wherein the concentration of IL-2 is at least 10 units/mL). After isolation of T lymphocytes, both cytotoxic and helper T lymphocytes can be sorted into naïve, memory, and effector T-cell subpopulations either before or after expansion.

As used herein, “genetically modified immune cells” or “Genetically engineered cells” are made by a process called genetic engineering, which can include but is not limited to manipulating a cells own genome or inserting a new nucleic acid into a cell. In some embodiments, these cells can be macrophages and can also be referred to as genetically engineered macrophages (GEMs). These techniques can be used to change the genetic makeup of the cell, and can include inserting a vector encoding a gene of interest into a cell, and genome editing using RNAi systems, meganucleases, zinc finger nucleases, transcription activator like effector nucleases (TALENs), or CRISPRs. Without being limiting, the vectors encoding the gene of interest can be a viral vector, DNA or an mRNA. In some embodiments, described herein, genetically modified immune cells are provided. In some embodiments, the genetically modified immune cells are made using genome editing proteins or systems, such as for example, meganucleases, zinc finger nucleases, transcription activator like effector nucleases (TALENs), CRISPR/VP64-Cas9 systems or CRISPR/CAS9 systems.

Some embodiments include polypeptide sequences or conservative variations thereof, such as conservative substitutions in a polypeptide sequence. In some embodiments, “conservative amino acid substitution” refers to amino acid substitutions that substitute functionally-equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. Substitutions that are charge neutral and which replace a residue with a smaller residue may also be considered “conservative substitutions” even if the residues are in different groups (e.g., replacement of phenylalanine with the smaller isoleucine). Families of amino acid residues having similar side chains have been defined in the art. Several families of conservative amino acid substitutions are shown in TABLE 1.

TABLE 1

| Family | Amino Acids |
|---|---|
| non-polar | Trp, Phe, Met, Leu, |
| | Ile, Val, Ala, Pro |
| uncharged polar | Gly, Ser, Thr, Asn, |
| | Gln, Tyr, Cys |
| acidic/negatively charged | Asp, Glu |
| basic/positively charged | Arg, Lys, His |
| Beta-branched | Thr, Val, Ile |
| residues that influence | Gly, Pro |
| chain orientation | |
| aromatic | Trp, Tyr, Phe, His |

### Certain Nucleic Acids

In some embodiments, the methods and compositions provided herein include a nucleic acid encoding a CAR, which is capable of or configured to specifically binding to an IL1RAP. In some embodiments, the CAR encoded by said nucleic acid comprises: a ligand binding domain, which specifically binds to IL1RAP, a spacer, a transmembrane domain, and an intracellular signalling domain.

In some embodiments, the nucleic acid comprises a first polynucleotide encoding a ligand binding domain. In some embodiments, the ligand binding domain encoded by said nucleic acid is derived from a binding moiety polypeptide derived from an expression library of antibodies or antibody fragments, such as Fab domains, V_{H} domains, and/or V_{L} domains. In some embodiments, the ligand binding domain encoded by said nucleic acid comprises a complementarity-determining region (CDR) derived from a binding moiety polypeptide, such as a binding moiety polypeptide, which specifically binds to an IL1RAP. In some embodiments, the binding moiety polypeptide is selected from 3A7, 4G6, or 3C5. In some embodiments, the ligand binding domain can include a heavy chain CDR1, a heavy chain CDR2, a heavy chain CDR3, a light chain CDR1, a light chain CDR2, and/or a light chain CDR3, derived from a binding moiety polypeptide, which specifically binds to an IL1RAP. In some embodiments, the ligand binding domain encoded by said nucleic acid comprises a CDR domain comprising or consisting of an amino acid sequence having 0-4 conservative amino acid substitutions of any one or more of SEQ ID NOs:42-53. In some embodiments, the ligand binding domain encoded by said nucleic acid comprises a CDR comprising the amino acid sequence having 0-4 conservative amino acid substitutions of any one or more of SEQ ID NOs:42-44, or 51-53. In some embodiments, the ligand binding domain encoded by said nucleic acid comprises a CDR3 comprising an amino acid sequence having 0-4 conservative amino acid substitutions of SEQ ID NO:44 or SEQ ID NO:53. In some embodiments, the ligand binding domain encoded by said nucleic acid comprises or consists of a CDR1 comprising an amino acid sequence having 0-4 conservative amino acid substitutions of SEQ ID NO:51; a CDR2 comprising or consisting of the amino acid sequence having 0-4 conservative amino acid substitutions of SEQ ID NO:52; and/or a CDR3 comprising or consisting of the amino acid sequence having 0-4 conservative amino acid substitutions of SEQ ID NO:44 or SEQ ID NO:53 or all three. In some embodiments, the ligand binding domain encoded by said nucleic acid comprises an amino acid sequence having at least or equal to 90%, 91%, 92%, 93%, 94% 95%, 96% 97%, 98%, 99%, or 100% identity to the amino acid sequence of any one of SEQ ID NOs:42-53. In some embodiments, the ligand binding domain encoded by said nucleic acid can include a variable heavy chain (VH) domain and/or a variable light chain (VL) domain, wherein the VH and/or VL domain is derived from a binding moiety polypeptide, which specifically binds to an IL1RAP. In some embodiments, the ligand binding domain encoded by said nucleic acid comprises a single chain variable fragment (scFv) derived from a binding moiety polypeptide which specifically binds to an IL1RAP. In some embodiments, the ligand binding domain encoded by said nucleic acid comprises a single chain variable fragment (scFv) derived from an optimized sequence from a binding moiety polypeptide, which specifically binds to an IL1RAP, such as a sequence of a CDR, a VH domain, and/or VL domain of the antibody. In some embodiments, the ligand binding domain encoded by said nucleic acid comprises an amino acid sequence having at least or equal to 90%, 91%, 92%, 93%, 94% 95%, 96% 97%, 98%, 99%, or 100% identity to the amino acid sequence of any one of SEQ ID NOs:01-04.

In some embodiments, the nucleic acid comprises a second polynucleotide encoding a spacer. In some embodiments, the spacer encoded by said nucleic acid comprises a CD8 spacer domain or an IgG4 hinge region. In some embodiments, the spacer encoded by said nucleic acid comprises a short (S), medium (M), or long (L) spacer. Examples of spacers are listed in TABLE 2.

In some embodiments, the nucleic acid comprises a third polynucleotide encoding a transmembrane domain. In some embodiments, the transmembrane domain encoded by said nucleic acid comprises a CD8 transmembrane domain. In some embodiments, the transmembrane domain encoded by said nucleic acid comprises an amino acid sequence having at least or equal to 90%, 91%, 92%, 93%, 94% 95%, 96% 97%, 98%, 99%, or 100% identity to the amino acid sequence of SEQ ID NO:13.

In some embodiments, the nucleic acid comprises a fourth polynucleotide encoding an intracellular signalling domain. In some embodiments, the intracellular signalling domain encoded by said nucleic acid comprises a costimulatory domain such as a CD27, CD28, 4-1BB, OX-40, CD30, CD40, PD-1, ICOS, LFA-1, CD2, CD7, NKG2C, or B7-H3. In some embodiments, the intracellular signalling domain encoded by said nucleic acid comprises a costimulatory domain in combination with a CD3 zeta domain or functional portion thereof. In some embodiments, the intracellular signalling domain encoded by said nucleic acid comprises a 4-1BB costimulatory domain. In some embodiments, the 4-1BB costimulatory domain encoded by said nucleic acid comprises an amino acid sequence having at least or equal to 90%, 91%, 92%, 93%, 94% 95%, 96% 97%, 98%, 99%, or 100% identity to the amino acid sequence of SEQ ID NO:15. In some embodiments, the CD3 zeta domain or functional portion thereof encoded by said nucleic acid comprises an amino acid sequence having at least or equal to 90%, 91%, 92%, 93%, 94% 95%, 96% 97%, 98%, 99%, or 100% identity to the amino acid sequence of SEQ ID NO:17.

In some embodiments, the nucleic acid comprises a fifth polynucleotide encoding a selectable marker. In some embodiments, the selectable marker encoded by said nucleic acid comprises a cell surface selectable marker. In some embodiments, the selectable marker encoded by said nucleic acid comprises a truncated EGFR polypeptide (EGFRt) or a truncated HER2 polypeptide (HER2t).

Some embodiments of the methods and compositions provided herein include a vector comprising any one of the nucleic acids provided herein. In some embodiments, the vector is a viral vector. In some embodiments, the vector is a lentiviral vector, foamy viral vector, retroviral vector, an adenoviral vector, or an adenovirus associated viral vector. In some embodiments, the vector is a transposon, integrase vector system, or an mRNA vector

### Certain CARs Specific for IL1RAP

Some embodiments of the methods and compositions provided herein include a CAR specific for IL1RAP. In some such embodiments, the CAR is encoded by a nucleic acid provided herein. The CAR can be encoded by the nucleic acid of any one of the embodiments herein or the vector of any one of the embodiments herein. Examples of polypeptide domains, which can be incorporated into any one or more of the CARs used in the products or methods described herein, are listed in TABLE 2.

TABLE 2

| Feature (SEQ ID NO) | Sequence |
|---|---|
| IL1RAP binding moiety | EVQLVESGGGLVQPGGSLRLSCAASGFTLSDYYMSW |
| 3A7 | VRQAPGKGLEWMGIIYPGDSDTRYSPSFQGHVTISRDN |
| Underlined: CDR1, CDR2, | SKNTLYLQMNSLRAEDTAVYYCTRWGAGMDVWGQ |
| CDR3, respectively | GTTVTVSS |
| (SEQ ID NO: 01) | |
| IL1RAP binding moiety | EVQLVESGGGLVQPGGSLRLSCAASGFTFSDYYMSW |
| 4G6 | VRQAPGQGLEWMGIIYPGDSDTRYSPSFQGQVTISRDN |
| Underlined: CDR1, CDR2, | SKNTLYLQMNSLRAEDTAVYYCVRWGAGIDVWGQG |
| CDR3, respectively | TTVTVSS |
| (SEQ ID NO: 02) | |
| IL1RAP binding moiety | EVQLVESGGGLVQSGGSLRLSCAASGFTFSDYYMSWV |
| 3C5 | RQAPGKALEWIGEINHSGSTNYNPSLKSLVTISRDNSK |
| Underlined: CDR1, CDR2, | NTLYLQMNSLRAEDTATYYCAREGDWYDAFDIWGQ |
| CDR3, respectively | GTTVTVSS |
| (SEQ ID NO: 03) | |
| IL1RAP binding moiety | EVQLVESGGGLVQPGGSLRLSCAASGFTLSDYYMSW |
| 7D12 | VRQAPGKGLEWMGIIYPGDSDTRYSPSFQGHVTISRDD |
| Underlined: CDR1, CDR2, | SKNTLYLQMNSLRAEDTAVYYCTRWGAGKDVWGQG |
| CDR3, respectively | TTVTVSS |
| (SEQ ID NO: 04) | |
| IL1RAP binding moiety | EVQLVESGGGLVQPGGSLRLSCAASGFTLSDYYMSWV |
| 3A7 | RQAPGKGLEWMGIIYPGDSDTRYSPSFQGHVTISRDNS |
| (SEQ ID NO: 05) | KNTLYLQMNSLRAEDTAVYYCTRWGAGMDVWGQGT |
| | TVTVSS |
| Nucleotide sequence | GAGGTGCAGCTGGTTGAATCTGGCGGAGGACTGGTT |
| encoding IL1RAP binding | CAGCCTGGCGGATCTCTGAGACTGTCTTGTGCCGCC |
| moiety 3A7 | AGCGGCTTCACCCTGAGCGACTACTATATGAGCTGG |
| (SEQ ID NO: 06) | GTCCGACAGGCCCCTGGCAAAGGACTTGAGTGGATG |
| | GGCATCATCTACCCCGGCGACAGCGACACCAGATAC |
| | AGCCCTAGCTTTCAGGGCCACGTGACCATCAGCCGG |
| | GACAACAGCAAGAACACCCTGTACCTGCAGATGAAC |
| | AGCCTGAGAGCCGAGGACACCGCCGTGTACTACTGT |
| | ACAAGATGGGGAGCCGGCATGGACGTGTGGGGACA |
| | GGGAACAACAGTGACCGTGTCTAGC |
| RJ104 CAR: (3A7/CD8 | MLLLVTSLLLCELPHPAFLLIPEVQLVESGGGLVQPGGS |
| TM/4-1BB/CD3zeta) | LRLSCAASGFTLSDYYMSWVRQAPGKGLEWMGIIYPG |
| (SEQ ID NO: 07) | DSDTRYSPSFQGHVTISRDNSKNTLYLQMNSLRAEDTA |
| | VYYCTRWGAGMDVWGQGTTVTVSSAAATTTPAPRPP |
| | TPAPTIASQPLSLRPEACRPAAGGAVHTRGLDFACDIYI |
| | WAPLAGTCGVLLLSLVITLYCKRGRKKLLYIFKQPFMR |
| | PVQTTQEEDGCSCRFPEEEEGGCELRVKFSRSADAPAY |
| | KQGQNQLYNELNLGRREEYDVLDKRRGRDPEMGGKP |
| | RRKNPQEGLYNELQKDKMAEAYSEIGMKGERRRGKG |
| | HDGLYQGLSTATKDTYDALHMQALPPR |
| Nucleotide sequence | ATGCTTCTGCTCGTGACAAGCCTGCTGCTGTGCGAG |
| encoding RJ104 CAR: | CTGCCCCACCCTGCCTTTCTGCTGATCCCTGAGGTGC |
| (3A7/CD8 TM/4- | AGCTGGTTGAATCTGGCGGAGGACTGGTTCAGCCTG |
| 1BB/CD3zeta) | GCGGATCTCTGAGACTGTCTTGTGCCGCCAGCGGCT |
| (SEQ ID NO: 08) | TCACCCTGAGCGACTACTATATGAGCTGGGTCCGAC |
| | AGGCCCCTGGCAAAGGACTTGAGTGGATGGGCATCA |
| | TCTACCCCGGCGACAGCGACACCAGATACAGCCCTA |
| | GCTTTCAGGGCCACGTGACCATCAGCCGGGACAACA |
| | GCAAGAACACCCTGTACCTGCAGATGAACAGCCTGA |
| | GAGCCGAGGACACCGCCGTGTACTACTGTACAAGAT |
| | GGGGAGCCGGCATGGACGTGTGGGGACAGGGAACA |
| | ACAGTGACCGTGTCTAGCGCGGCCGCAACCACTACT |
| | CCGGCACCTAGGCCCCCCACTCCGGCACCGACCATT |
| | GCATCACAACCACTGAGTCTTAGACCTGAAGCCTGT |
| | CGACCCGCAGCTGGGGGGGCAGTCCACACACGGGG |
| | ATTGGATTTCGCCTGCGATATATACATTTGGGCGCCA |
| | CTGGCAGGCACCTGCGGGGTCCTGCTCTTGTCCCTTG |
| | TCATCACCCTGTACTGTAAGAGAGGAAGAAAGAAGT |
| | TGTTGTATATTTTCAAACAGCCATTCATGAGGCCGGT |
| | CCAGACCACTCAGGAGGAGGATGGATGCAGTTGCCG |
| | CTTTCCGGAAGAGGAAGAGGGTGGGTGTGAACTCCG |
| | AGTTAAATTTTCTCGCAGTGCTGATGCCCCAGCGTAT |
| | AAACAGGGTCAGAACCAACTGTATAATGAACTCAAT |
| | TTGGGGAGGCGAGAGGAGTATGATGTATTGGACAA |
| | GCGGCGGGGGCGGGATCCTGAAATGGGCGGTAAGC |
| | CAAGGCGCAAGAATCCACAGGAGGGTTTGTATAATG |
| | AGTTGCAGAAGGATAAAATGGCGGAAGCGTATTCA |
| | GAAATCGGAATGAAGGGAGAGCGCCGCAGAGGGAA |
| | AGGGCATGATGGCCTCTACCAAGGTTTGTCAACAGC |
| | GACCAAGGATACCTATGACGCACTTCATATGCAAGC |
| | ACTGCCCCCCAGG |
| Leader signal polypeptide | MLLLVTSLLLCELPHPAFLLIP |
| (SEQ ID NO: 09) | |
| Nucleotide sequence | ATGCTTCTGCTCGTGACAAGCCTGCTGCTGTGCGAG |
| encoding leader signal | CTGCCCCACCCTGCCTTTCTGCTGATCCCT |
| polypeptide | |
| (SEQ ID NO: 10) | |
| Linker | AAA |
| (SEQ ID NO: 11) | |
| Nucleotide sequence | GCGGCCGCA |
| encoding linker | |
| (SEQ ID NO: 12) | |
| CD8 linker and | TTTPAPRPPTPAPTIASQPLSLRPEACRPAAGGAVHTRG |
| transmembrane | LDFACDIYIWAPLAGTCGVLLLSLVITLYC |
| (SEQ ID NO: 13) | |
| Nucleotide sequence | ACCACTACTCCGGCACCTAGGCCCCCCACTCCGGCA |
| encoding CD8 linker and | CCGACCATTGCATCACAACCACTGAGTCTTAGACCT |
| transmembrane | GAAGCCTGTCGACCCGCAGCTGGGGGGGCAGTCCAC |
| (SEQ ID NO: 14) | ACACGGGGATTGGATTTCGCCTGCGATATATACATT |
| | TGGGCGCCACTGGCAGGCACCTGCGGGGTCCTGCTC |
| | TTGTCCCTTGTCATCACCCTGTACTGT |
| 4-1BB signaling domain | KRGRKKLLYIFKQPFMRPVQTTQEEDGCSCRFPEEEEG |
| (SEQ ID NO: 15) | GCEL |
| Nucleotide sequence | AAGAGAGGAAGAAAGAAGTTGTTGTATATTTTCAAA |
| encoding 4-1BB signaling | CAGCCATTCATGAGGCCGGTCCAGACCACTCAGGAG |
| domain | GAGGATGGATGCAGTTGCCGCTTTCCGGAAGAGGAA |
| (SEQ ID NO: 16) | GAGGGTGGGTGTGAACTC |
| CD3-zeta chain signaling | RVKFSRSADAPAYKQGQNQLYNELNLGRREEYDVLD |
| domain | KRRGRDPEMGGKPRRKNPQEGLYNELQKDKMAEAYS |
| (SEQ ID NO: 17) | EIGMKGERRRGKGHDGLYQGLSTATKDTYDALHMQA |
| | LPPR |
| Nucleotide sequence | CGAGTTAAATTTTCTCGCAGTGCTGATGCCCCAGCGT |
| encoding CD3-zeta chain | ATAAACAGGGTCAGAACCAACTGTATAATGAACTCA |
| signaling domain | ATTTGGGGAGGCGAGAGGAGTATGATGTATTGGACA |
| (SEQ ID NO: 18) | AGCGGCGGGGGCGGGATCCTGAAATGGGCGGTAAG |
| | CCAAGGCGCAAGAATCCACAGGAGGGTTTGTATAAT |
| | GAGTTGCAGAAGGATAAAATGGCGGAAGCGTATTC |
| | AGAAATCGGAATGAAGGGAGAGCGCCGCAGAGGGA |
| | AAGGGCATGATGGCCTCTACCAAGGTTTGTCAACAG |
| | CGACCAAGGATACCTATGACGCACTTCATATGCAAG |
| | CACTGCCCCCCAGG |
| IL1RAP binding moiety | EVQLVESGGGLVQPGGSLRLSCAASGFTFSDYYMSWV |
| 4G6 | RQAPGQGLEWMGIIYPGDSDTRYSPSFQGQVTISRDNS |
| (SEQ ID NO: 19) | KNTLYLQMNSLRAEDTAVYYCVRWGAGIDVWGQGT |
| | TVTVSS |
| Nucleotide sequence | GAGGTGCAGCTGGTTGAATCTGGCGGAGGACTGGTT |
| encoding IL1RAP binding | CAGCCTGGCGGATCTCTGAGACTGTCTTGTGCCGCC |
| moiety 4G6 | AGCGGCTTCACCTTCAGCGACTACTACATGAGCTGG |
| (SEQ ID NO: 20) | GTCCGACAGGCCCCTGGACAAGGACTTGAGTGGATG |
| | GGCATCATCTACCCCGGCGACAGCGACACCAGATAC |
| | AGCCCTAGCTTTCAGGGCCAAGTGACCATCAGCCGG |
| | GACAACAGCAAGAACACCCTGTACCTGCAGATGAAC |
| | AGCCTGAGAGCCGAGGACACCGCCGTGTACTACTGT |
| | GTTAGATGGGGAGCCGGCATCGACGTGTGGGGACA |
| | GGGAACAACAGTGACCGTGTCTAGC |
| RJ105 CAR (4G6/CD8 | MLLLVTSLLLCELPHPAFLLIPEVQLVESGGGLVQPGGS |
| TM/4-1BB/CD3zeta) | LRLSCAASGFTFSDYYMSWVRQAPGQGLEWMGIIYPG |
| (SEQ ID NO: 21) | DSDTRYSPSFQGQVTISRDNSKNTLYLQMNSLRAEDTA |
| | VYYCVRWGAGIDVWGQGTTVTVSSAAATTTPAPRPPT |
| | PAPTIASQPLSLRPEACRPAAGGAVHTRGLDFACDIYIW |
| | APLAGTCGVLLLSLVITLYCKRGRKKLLYIFKQPFMRP |
| | VQTTQEEDGCSCRFPEEEEGGCELRVKFSRSADAPAYK |
| | QGQNQLYNELNLGRREEYDVLDKRRGRDPEMGGKPR |
| | RKNPQEGLYNELQKDKMAEAYSEIGMKGERRRGKGH |
| | DGLYQGLSTATKDTYDALHMQALPPR |
| Nucleotide sequence | ATGCTTCTGCTCGTGACAAGCCTGCTGCTGTGCGAG |
| encoding RJ105 CAR | CTGCCCCACCCTGCCTTTCTGCTGATCCCTGAGGTGC |
| (4G6/CD8 TM/4- | AGCTGGTTGAATCTGGCGGAGGACTGGTTCAGCCTG |
| 1BB/CD3zeta) | GCGGATCTCTGAGACTGTCTTGTGCCGCCAGCGGCT |
| (SEQ ID NO: 22) | TCACCTTCAGCGACTACTACATGAGCTGGGTCCGAC |
| | AGGCCCCTGGACAAGGACTTGAGTGGATGGGCATCA |
| | TCTACCCCGGCGACAGCGACACCAGATACAGCCCTA |
| | GCTTTCAGGGCCAAGTGACCATCAGCCGGGACAACA |
| | GCAAGAACACCCTGTACCTGCAGATGAACAGCCTGA |
| | GAGCCGAGGACACCGCCGTGTACTACTGTGTTAGAT |
| | GGGGAGCCGGCATCGACGTGTGGGGACAGGGAACA |
| | ACAGTGACCGTGTCTAGCGCGGCCGCAACCACTACT |
| | CCGGCACCTAGGCCCCCCACTCCGGCACCGACCATT |
| | GCATCACAACCACTGAGTCTTAGACCTGAAGCCTGT |
| | CGACCCGCAGCTGGGGGGGCAGTCCACACACGGGG |
| | ATTGGATTTCGCCTGCGATATATACATTTGGGCGCCA |
| | CTGGCAGGCACCTGCGGGGTCCTGCTCTTGTCCCTTG |
| | TCATCACCCTGTACTGTAAGAGAGGAAGAAAGAAGT |
| | TGTTGTATATTTTCAAACAGCCATTCATGAGGCCGGT |
| | CCAGACCACTCAGGAGGAGGATGGATGCAGTTGCCG |
| | CTTTCCGGAAGAGGAAGAGGGTGGGTGTGAACTCCG |
| | AGTTAAATTTTCTCGCAGTGCTGATGCCCCAGCGTAT |
| | AAACAGGGTCAGAACCAACTGTATAATGAACTCAAT |
| | TTGGGGAGGCGAGAGGAGTATGATGTATTGGACAA |
| | GCGGCGGGGGCGGGATCCTGAAATGGGCGGTAAGC |
| | CAAGGCGCAAGAATCCACAGGAGGGTTTGTATAATG |
| | AGTTGCAGAAGGATAAAATGGCGGAAGCGTATTCA |
| | GAAATCGGAATGAAGGGAGAGCGCCGCAGAGGGAA |
| | AGGGCATGATGGCCTCTACCAAGGTTTGTCAACAGC |
| | GACCAAGGATACCTATGACGCACTTCATATGCAAGC |
| | ACTGCCCCCCAG |
| IL1RAP binding moiety | EVQLVESGGGLVQSGGSLRLSCAASGFTFSDYYMSWV |
| 3C5 | RQAPGKALEWIGEINHSGSTNYNPSLKSLVTISRDNSK |
| (SEQ ID NO: 23) | NTLYLQMNSLRAEDTATYYCAREGDWYDAFDIWGQG |
| | TTVTVSS |
| Nucleotide sequence | GAGGTGCAGCTGGTTGAATCTGGCGGAGGACTGGTT |
| encoding IL1RAP binding | CAGTCTGGCGGCTCTCTGAGACTGTCTTGTGCCGCCA |
| moiety 3C5 | GCGGCTTCACCTTCAGCGACTACTACATGAGCTGGG |
| (SEQ ID NO: 24) | TCCGACAGGCCCCTGGAAAAGCCCTGGAATGGATCG |
| | GCGAGATCAACCACAGCGGCAGCACCAACTACAAC |
| | CCCAGCCTGAAGTCCCTGGTCACCATCAGCAGAGAC |
| | AACAGCAAGAACACCCTGTACCTGCAGATGAACAGC |
| | CTGAGAGCCGAGGACACCGCCACCTACTACTGTGCC |
| | AGAGAAGGCGATTGGTACGACGCCTTCGATATCTGG |
| | GGCCAGGGCACCACAGTGACCGTTTCTTCT |
| RJ106 CAR (3C5/CD8 | MLLLVTSLLLCELPHPAFLLIPEVQLVESGGGLVQSGGS |
| TM/4-1BB/CD3zeta) | LRLSCAASGFTFSDYYMSWVRQAPGKALEWIGEINHS |
| (SEQ ID NO: 25) | GSTNYNPSLKSLVTISRDNSKNTLYLQMNSLRAEDTAT |
| | YYCAREGDWYDAFDIWGQGTTVTVSSAAATTTPAPRP |
| | PTPAPTIASQPLSLRPEACRPAAGGAVHTRGLDFACDIY |
| | IWAPLAGTCGVLLLSLVITLYCKRGRKKLLYIFKQPFM |
| | RPVQTTQEEDGCSCRFPEEEEGGCELRVKFSRSADAPA |
| | YKQGQNQLYNELNLGRREEYDVLDKRRGRDPEMGGK |
| | PRRKNPQEGLYNELQKDKMAEAYSEIGMKGERRRGK |
| | GHDGLYQGLSTATKDTYDALHMQALPPR |
| Nucleotide sequence | ATGCTTCTGCTCGTGACAAGCCTGCTGCTGTGCGAG |
| encoding RJ106 CAR | CTGCCCCACCCTGCCTTTCTGCTGATCCCTGAGGTGC |
| (3C5/CD8 TM/4- | AGCTGGTTGAATCTGGCGGAGGACTGGTTCAGTCTG |
| 1BB/CD3zeta) | GCGGCTCTCTGAGACTGTCTTGTGCCGCCAGCGGCTT |
| (SEQ ID NO: 26) | CACCTTCAGCGACTACTACATGAGCTGGGTCCGACA |
| | GGCCCCTGGAAAAGCCCTGGAATGGATCGGCGAGAT |
| | CAACCACAGCGGCAGCACCAACTACAACCCCAGCCT |
| | GAAGTCCCTGGTCACCATCAGCAGAGACAACAGCAA |
| | GAACACCCTGTACCTGCAGATGAACAGCCTGAGAGC |
| | CGAGGACACCGCCACCTACTACTGTGCCAGAGAAGG |
| | CGATTGGTACGACGCCTTCGATATCTGGGGCCAGGG |
| | CACCACAGTGACCGTTTCTTCTGCGGCCGCAACCAC |
| | TACTCCGGCACCTAGGCCCCCCACTCCGGCACCGAC |
| | CATTGCATCACAACCACTGAGTCTTAGACCTGAAGC |
| | CTGTCGACCCGCAGCTGGGGGGGCAGTCCACACACG |
| | GGGATTGGATTTCGCCTGCGATATATACATTTGGGC |
| | GCCACTGGCAGGCACCTGCGGGGTCCTGCTCTTGTC |
| | CCTTGTCATCACCCTGTACTGTAAGAGAGGAAGAAA |
| | GAAGTTGTTGTATATTTTCAAACAGCCATTCATGAG |
| | GCCGGTCCAGACCACTCAGGAGGAGGATGGATGCA |
| | GTTGCCGCTTTCCGGAAGAGGAAGAGGGTGGGTGTG |
| | AACTCCGAGTTAAATTTTCTCGCAGTGCTGATGCCCC |
| | AGCGTATAAACAGGGTCAGAACCAACTGTATAATGA |
| | ACTCAATTTGGGGAGGCGAGAGGAGTATGATGTATT |
| | GGACAAGCGGCGGGGGCGGGATCCTGAAATGGGCG |
| | GTAAGCCAAGGCGCAAGAATCCACAGGAGGGTTTGT |
| | ATAATGAGTTGCAGAAGGATAAAATGGCGGAAGCG |
| | TATTCAGAAATCGGAATGAAGGGAGAGCGCCGCAG |
| | AGGGAAAGGGCATGATGGCCTCTACCAAGGTTTGTC |
| | AACAGCGACCAAGGATACCTATGACGCACTTCATAT |
| | GCAAGCACTGCCCCCCAGG |
| IL1RAP binding moiety | EVQLVESGGGLVQPGGSLRLSCAASGFTLSDYYMSWV |
| 7D12 | RQAPGKGLEWMGIIYPGDSDTRYSPSFQGHVTISRDDS |
| (SEQ ID NO: 27) | KNTLYLQMNSLRAEDTAVYYCTRWGAGKDVWGQGT |
| | TVTVSS |
| Nucleotide sequence | GAGGTGCAGCTGGTTGAATCTGGCGGAGGACTGGTT |
| encoding IL1RAP binding | CAGCCTGGCGGATCTCTGAGACTGTCTTGTGCCGCC |
| moiety 7D12 | AGCGGCTTCACCCTGAGCGACTACTATATGAGCTGG |
| (SEQ ID NO: 28) | GTCCGACAGGCCCCTGGCAAAGGACTTGAGTGGATG |
| | GGCATCATCTACCCCGGCGACAGCGACACCAGATAC |
| | AGCCCTAGCTTTCAGGGCCACGTGACCATCAGCCGG |
| | GACGACAGCAAGAACACCCTGTACCTGCAGATGAAC |
| | AGCCTGAGAGCCGAGGACACCGCCGTGTACTACTGT |
| | ACAAGATGGGGAGCCGGCAAGGACGTGTGGGGACA |
| | GGGAACAACAGTGACCGTGTCTAGC |
| RJ107 CAR (7D12/CD8 | MLLLVTSLLLCELPHPAFLLIPEVQLVESGGGLVQPGGS |
| TM/4-1BB/CD3zeta) | LRLSCAASGFTLSDYYMSWVRQAPGKGLEWMGIIYPG |
| (SEQ ID NO: 29) | DSDTRYSPSFQGHVTISRDDSKNTLYLQMNSLRAEDTA |
| | VYYCTRWGAGKDVWGQGTTVTVSSAAATTTPAPRPP |
| | TPAPTIASQPLSLRPEACRPAAGGAVHTRGLDFACDIYI |
| | WAPLAGTCGVLLLSLVITLYCKRGRKKLLYIFKQPFMR |
| | PVQTTQEEDGCSCRFPEEEEGGCELRVKFSRSADAPAY |
| | KQGQNQLYNELNLGRREEYDVLDKRRGRDPEMGGKP |
| | RRKNPQEGLYNELQKDKMAEAYSEIGMKGERRRGKG |
| | HDGLYQGLSTATKDTYDALHMQALPPR |
| Nucleotide sequence | ATGCTTCTGCTCGTGACAAGCCTGCTGCTGTGCGAG |
| encoding RJ107 CAR | CTGCCCCACCCTGCCTTTCTGCTGATCCCTGAGGTGC |
| (7D12/CD8 TM/4- | AGCTGGTTGAATCTGGCGGAGGACTGGTTCAGCCTG |
| 1BB/CD3zeta) | GCGGATCTCTGAGACTGTCTTGTGCCGCCAGCGGCT |
| (SEQ ID NO: 30) | TCACCCTGAGCGACTACTATATGAGCTGGGTCCGAC |
| | AGGCCCCTGGCAAAGGACTTGAGTGGATGGGCATCA |
| | TCTACCCCGGCGACAGCGACACCAGATACAGCCCTA |
| | GCTTTCAGGGCCACGTGACCATCAGCCGGGACGACA |
| | GCAAGAACACCCTGTACCTGCAGATGAACAGCCTGA |
| | GAGCCGAGGACACCGCCGTGTACTACTGTACAAGAT |
| | GGGGAGCCGGCAAGGACGTGTGGGGACAGGGAACA |
| | ACAGTGACCGTGTCTAGCGCGGCCGCAACCACTACT |
| | CCGGCACCTAGGCCCCCCACTCCGGCACCGACCATT |
| | GCATCACAACCACTGAGTCTTAGACCTGAAGCCTGT |
| | CGACCCGCAGCTGGGGGGGCAGTCCACACACGGGG |
| | ATTGGATTTCGCCTGCGATATATACATTTGGGCGCCA |
| | CTGGCAGGCACCTGCGGGGTCCTGCTCTTGTCCCTTG |
| | TCATCACCCTGTACTGTAAGAGAGGAAGAAAGAAGT |
| | TGTTGTATATTTTCAAACAGCCATTCATGAGGCCGGT |
| | CCAGACCACTCAGGAGGAGGATGGATGCAGTTGCCG |
| | CTTTCCGGAAGAGGAAGAGGGTGGGTGTGAACTCCG |
| | AGTTAAATTTTCTCGCAGTGCTGATGCCCCAGCGTAT |
| | AAACAGGGTCAGAACCAACTGTATAATGAACTCAAT |
| | TTGGGGAGGCGAGAGGAGTATGATGTATTGGACAA |
| | GCGGCGGGGGCGGGATCCTGAAATGGGCGGTAAGC |
| | CAAGGCGCAAGAATCCACAGGAGGGTTTGTATAATG |
| | AGTTGCAGAAGGATAAAATGGCGGAAGCGTATTCA |
| | GAAATCGGAATGAAGGGAGAGCGCCGCAGAGGGAA |
| | AGGGCATGATGGCCTCTACCAAGGTTTGTCAACAGC |
| | GACCAAGGATACCTATGACGCACTTCATATGCAAGC |
| | ACTGCCCCCCAGG |
| scFv linker | GGGGSGGGGSGGGGS |
| (SEQ ID NO: 31) | |
| S spacer | ESKYGPPCPPCP |
| (SEQ ID NO: 32) | |
| Nucleotide sequence | GAATCTAAGTACGGACCGCCCTGCCCCCCTTGCCCT |
| encoding a S spacer | |
| (SEQ ID NO: 33) | |
| M spacer | ESKYGPPCPPCPGQPREPQVYTLPPSQEEMTKNQVSLT |
| (SEQ ID NO: 34) | CLVKGFYPSDIAVEWESNGQPENNYKTTPPVLDSDGSF |
| | FLYSRLTVDKSRWQEGNVFSCSVMHEALHNHYTQKSL |
| | SLSLGK |
| Nucleotide sequence | GAATCTAAGTACGGACCGCCCTGCCCCCCTTGCCCT |
| encoding a M spacer | GGCCAGCCTAGAGAACCCCAGGTGTACACCCTGCCT |
| (SEQ ID NO: 35) | CCCAGCCAGGAAGAGATGACCAAGAACCAGGTGTC |
| | CCTGACCTGCCTGGTCAAAGGCTTCTACCCCAGCGA |
| | TATCGCCGTGGAATGGGAGAGCAACGGCCAGCCCG |
| | AGAACAACTACAAGACCACCCCCCCTGTGCTGGACA |
| | GCGACGGCAGCTTCTTCCTGTACTCCCGGCTGACCGT |
| | GGACAAGAGCCGGTGGCAGGAAGGCAACGTCTTCA |
| | GCTGCAGCGTGATGCACGAGGCCCTGCACAACCACT |
| | ACACCCAGAAGTCCCTGAGCCTGAGCCTGGGCAAG |
| L spacer | ESKYGPPCPPCPAPEFLGGPSVFLFPPKPKDTLMISRTPE |
| (SEQ ID NO: 36) | VTCVVVDVSQEDPEVQFNWYVDGVEVHNAKTKPREE |
| | QFNSTYRVVSVLTVLHQDWLNGKEYKCKVSNKGLPSS |
| | IEKTISKAKGQPREPQVYTLPPSQEEMTKNQVSLTCLV |
| | KGFYPSDIAVEWESNGQPENNYKTTPPVLDSDGSFFLY |
| | SRLTVDKSRWQEGNVFSCSVMHEALHNHYTQKSLSLS |
| | LGK |
| Nucleotide sequence | ATCTAAGTACGGACCGCCCTGCCCCCCTTGCCCTGCC |
| encoding a L spacer | CCCGAGTTCGACGGCGGACCCAGCGTGTTCCTGTTC |
| (SEQ ID NO: 37) | CCCCCCAAGCCCAAGGACACCCTGATGATCAGCCGG |
| | ACCCCCGAGGTGACCTGCGTGGTGGTGGACGTGAGC |
| | CAGGAAGATCCCGAGGTCCAGTTCAATTGGTACGTG |
| | GACGGCGTGGAAGTGCACAACGCCAAGACCAAGCC |
| | CAGAGAGGAACAGTTCCAGAGCACCTACCGGGTGGT |
| | GTCTGTGCTGACCGTGCTGCACCAGGACTGGCTGAA |
| | CGGCAAAGAATACAAGTGCAAGGTGTCCAACAAGG |
| | GCCTGCCCAGCAGCATCGAAAAGACCATCAGCAAG |
| | GCCAAGGGCCAGCCTCGCGAGCCCCAGGTGTACACC |
| | CTGCCTCCCTCCCAGGAAGAGATGACCAAGAACCAG |
| | GTGTCCCTGACCTGCCTGGTGAAGGGCTTCTACCCC |
| | AGCGACATCGCCGTGGAGTGGGAGAGCAACGGCCA |
| | GCCTGAGAACAACTACAAGACCACCCCTCCCGTGCT |
| | GGACAGCGACGGCAGCTTCTTCCTGTACAGCCGGCT |
| | GACCGTGGACAAGAGCCGGTGGCAGGAAGGCAACG |
| | TCTTTAGCTGCAGCGTGATGCACGAGGCCCTGCACA |
| | ACCACTACACCCAGAAGAGCCTGAGCCTGTCCCTGG |
| | GCAAG |
| CD28tm | MFWVLVVVGGVLACYSLLVTVAFIIFWV |
| (SEQ ID NO: 38) | |
| Nucleotide sequence | ATGTTCTGGGTGCTGGTGGTGGTCGGAGGCGTGCTG |
| encoding a CD28tm | GCCTGCTACAGCCTGCTGGTCACCGTGGCCTTCATCA |
| (SEQ ID NO: 39) | TCTTTTGGGTG |
| T2A | CTCGAGGGCGGCGGAGAGGGCAGAGGAAGTCTTCT |
| (SEQ ID NO: 40) | AACATGCGGTGACGTGGAGGAGAATCCCGGCCCTAG |
| | G |
| Nucleotide sequence | CGCAAAGTGTGTAACGGAATAGGTATTGGTGAATTT |
| encoding an EGFRt | AAAGACTCACTCTCCATAAATGCTACGAATATTAAA |
| (SEQ ID NO: 41) | CACTTCAAAAACTGCACCTCCATCAGTGGCGATCTC |
| | CACATCCTGCCGGTGGCATTTAGGGGTGACTCCTTC |
| | ACACATACTCCTCCTCTGGATCCACAGGAACTGGAT |
| | ATTCTGAAAACCGTAAAGGAAATCACAGGGTTTTTG |
| | CTGATTCAGGCTTGGCCTGAAAACAGGACGGACCTC |
| | CATGCCTTTGAGAACCTAGAAATCATACGCGGCAGG |
| | ACCAAGCAACATGGTCAGTTTTCTCTTGCAGTCGTCA |
| | GCCTGAACATAACATCCTTGGGATTACGCTCCCTCA |
| | AGGAGATAAGTGATGGAGATGTGATAATTTCAGGAA |
| | ACAAAAATTTGTGCTATGCAAATACAATAAACTGGA |
| | AAAAACTGTTTGGGACCTCCGGTCAGAAAACCAAAA |
| | TTATAAGCAACAGAGGTGAAAACAGCTGCAAGGCC |
| | ACAGGCCAGGTCTGCCATGCCTTGTGCTCCCCCGAG |
| | GGCTGCTGGGGCCCGGAGCCCAGGGACTGCGTCTCT |
| | TGCCGGAATGTCAGCCGAGGCAGGGAATGCGTGGA |
| | CAAGTGCAACCTTCTGGAGGGTGAGCCAAGGGAGTT |
| | TGTGGAGAACTCTGAGTGCATACAGTGCCACCCAGA |
| | GTGCCTGCCTCAGGCCATGAACATCACCTGCACAGG |
| | ACGGGGACCAGACAACTGTATCCAGTGTGCCCACTA |
| | CATTGACGGCCCCCACTGCGTCAAGACCTGCCCGGC |
| | AGGAGTCATGGGAGAAAACAACACCCTGGTCTGGA |
| | AGTACGCAGACGCCGGCCATGTGTGCCACCTGTGCC |
| | ATCCAAACTGCACCTACGGATGCACTGGGCCAGGTC |
| | TTGAAGGCTGTCCAACGAATGGGCCTAAGATCCCGT |
| | CCATCGCCACTGGGATGGTGGGGGCCCTCCTCTTGC |
| | TGCTGGTGGTGGCCCTGGGGATCGGCCTCTTCATG |
| IL1RAP binding moiety | GFTLSDYY |
| 3A7, CDR1 | |
| (SEQ ID NO: 42) | |
| IL1RAP binding moiety | IYPGDSDT |
| 3A7, CDR2 | |
| (SEQ ID NO: 43) | |
| IL1RAP binding moiety | TRWGAGMDV |
| 3A7, CDR3 | |
| (SEQ ID NO: 44) | |
| IL1RAP binding moiety | GFTFSDYYM |
| 4G6, CDR1 | |
| (SEQ ID NO: 45) | |
| IL1RAP binding moiety | IYPGDSDT |
| 4G6, CDR2 | |
| (SEQ ID NO: 46) | |
| IL1RAP binding moiety | VRWGAGIDV |
| 4G6, CDR3 | |
| (SEQ ID NO: 47) | |
| IL1RAP binding moiety | GFTFSDYY |
| 3C5, CDR1 | |
| (SEQ ID NO: 48) | |
| IL1RAP binding moiety | INHSGST |
| 3C5, CDR2 | |
| (SEQ ID NO: 49) | |
| IL1RAP binding moiety | AREGDWYDAFDI |
| 3C5, CDR3 | |
| (SEQ ID NO: 50) | |
| IL1RAP binding moiety | GFTLSDYY |
| 7D12, CDR1 | |
| (SEQ ID NO: 51) | |
| IL1RAP binding moiety | IYPGDSDT |
| 7D12, CDR2 | |
| (SEQ ID NO: 52) | |
| IL1RAP binding moiety | TRWGAGKDV |
| 7D12, CDR3 | |
| (SEQ ID NO: 53) | |

### Certain Cells for CAR T Cell Expression and Therapy

Some embodiments of the methods and compositions provided herein include a cell comprising a CAR as described in any one or more of the embodiments herein. In some embodiments, the cell is from a donor that is related or unrelated to a subject in need of CAR T cell therapy. In some embodiments, the cell is from the subject in need of therapy. In some embodiments, the cell is a CD4+ T-cell or a CD8+ T-cell. In some embodiments, the cell is a precursor T-cell, or a hematopoietic stem cell. In some embodiments, the cell is a CD8+ cytotoxic T-cell selected from the group consisting of a naïve CD8+ T-cell, a CD8+ memory T-cell, a central memory CD8+ T-cell, a regulatory CD8+ T-cell, an IPS derived CD8+ T-cell, an effector memory CD8+ T-cell, and a bulk CD8+ T-cell. In some embodiments, the cell is a CD4+T helper cell selected from the group consisting of a naïve CD4+ T-cell, a CD4+ memory T-cell, a central memory CD4+ T-cell, a regulatory CD4+ T-cell, an IPS derived CD4+ T-cell, an effector memory CD4+ T-cell, and a bulk CD4+ T-cell.

### Certain Methods of Preparing Donor Cells

Some embodiments of the methods and compositions provided herein include methods of preparing a population of cells comprising a CAR specific for IL1RAP, such as a population of cells for an infusion. Some embodiments include obtaining a cell from a subject, or a cell from another matched to be compatible to the subject for cellular therapy. Some embodiments also include introducing any one of the vectors provided herein containing a CAR specific for IL1RAP into the cell. Some embodiments also include expanding the cell and isolating the cell. Some embodiments include culturing the cell in the presence of an agent selected from an anti-CD3, an anti-CD28, or a cytokine, such as IL-2.

### Certain Methods of Therapy

Some embodiments of the methods and compositions provided herein include methods of therapy, such as methods of treating, inhibiting or ameliorating a cancer in a subject. In some embodiments, the cancer comprises a cancer cell expressing IL1RAP. In some embodiments, the cancer is selected from the group consisting of a breast cancer, a brain cancer, a colon cancer, a renal cancer, a pancreatic cancer, an ovarian cancer, a sarcoma, and a leukemia. In some embodiments, the cancer comprises an acute myeloid leukemia (AML), a chronic myelogenous leukemia (CVL), and a Ewing's sarcoma. In some embodiments, the subject is mammalian. In some embodiments, the subject is human.

### EXAMPLES

### Example 1-Construction of CARs

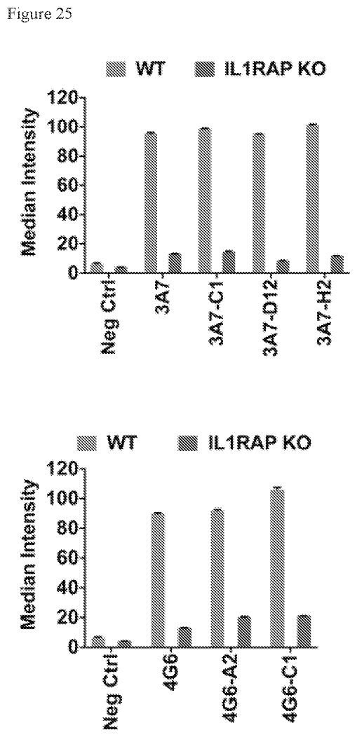

IL1RAP binding moieties were derived from phage or yeast display libraries encoding human immunoglobulin derived sequences in different formats including Fab and VH domains. The libraries were screened against recombinant IL1RAP. Identified candidates were cloned into immunoglobulin expression vectors, and binding activity of the expressed binding moiety was determined. Identified binding moieties included: 3A7, 4G6, and 3C5. A further IL1RAP binding moiety, 7D12, was derived from a second library affinity optimization screen of a 3A7-enriched library. All four binding moieties bound recombinant IL1RAP when expressed as recombinant immunoglobulins. 3A7 and 4G6 were tested using wild-type and IL1RAP knock out cells and exhibited binding to the wild-type cells (FIG. 25).

Nucleic acids encoding CARs were constructed: RJ104 CAR contained the 3A7 binding moiety; RJ105 CAR contained the 4G6 binding moiety; RJ106 CAR contained the 3C5 binding moiety; and RJ107 CAR contained the 7D12 binding moiety. Nucleic acids encoding CARs were cloned into lentiviral vectors. FIG. 1 depicts an example structure of a nucleic acid encoding a CAR and includes: lentiviral vector elements including a 5′ LTR and a 3′ LTR; a promoter, such as an E1α promoter; and polynucleotides encoding a ligand binding domain (binder), a spacer (linker), a CD8 transmembrane domain, a 4-1BB domain, and a CD3-zeta domain. TABLE 2 lists sequences used to construct the CARs.

### Example 2-Cell Surface Expression of CARs

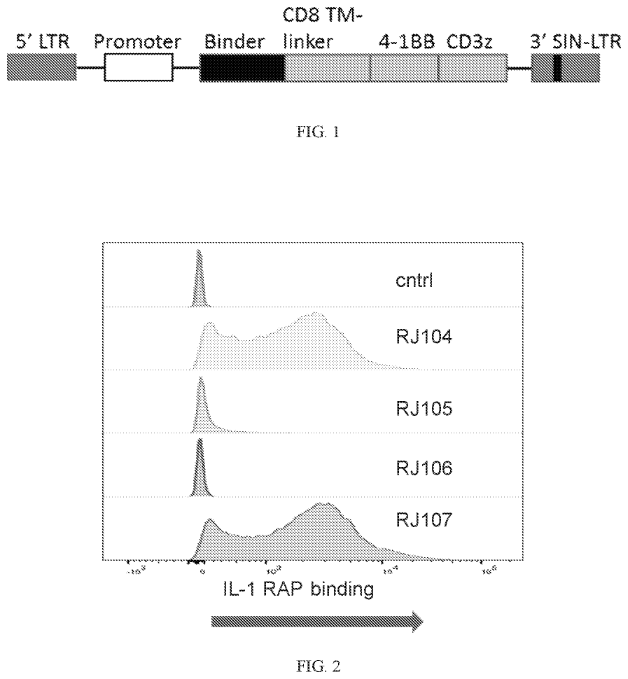

Lentiviral vectors encoding CARs were titrated and used to transduce human T cells. Cell surface expression of the CARs was determined by contacting the cells with a recombinant biotinylated IL1RAP, washing unbound biotinylated IL1RAP from the contacted cells, and detecting the bound biotinylated IL1RAP by incubating the cells with streptavidin-linked-phycoerythrin (SA-PE). Unbound SA-PE was washed from the cells, and the cells analyzed by flow cytometry. FIG. 2 depicts the results and showed that cells exposed only to SA-PE showed no binding (x-axis); cells containing the RJ105 CAR or the RJ106 CAR showed essentially no binding in a CAR format; however, cells containing the RJ104 CAR or the RJO17 CAR strongly bound the target antigen, IL1RAP.

### Example 3 In Vitro Activity of CARs Against Ewing's Sarcoma Cells

Human T cells were activated in the presence of IL-2, and then transduced with lentiviral vectors encoding either the RJ104 CAR (pRJ104), the RJ105 CAR (pRJ105), the RJ106 CAR (pRJ106), or the RJ107 CAR (pRJ107). Control cells include activated cells (activated T cells). A Ewing's sarcoma line (target cells), TC71, which expressed the target antigen IL1RAP and a luciferase marker was co-cultured with the T cells (effector cells) at various effector to target ratios (E:T ratio). Specific lysis of the target cells was measured.

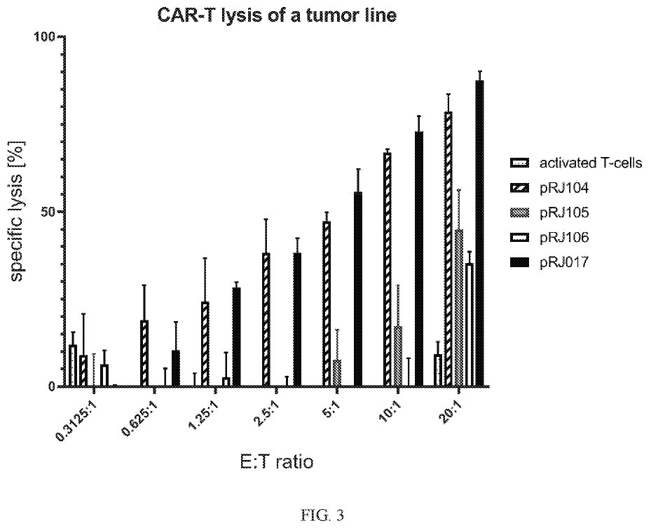

As depicted in FIG. 3, T cells containing either the RJ104 CAR or RJ107 CAR readily lysed target TC71 cells at lower E:T ratios. In contrast, T cells containing either the RJ105 CAR or RJ106 CAR did not readily lyse target TC71 cells at lower E:T ratios. Thus, the sequences of the RJ104 CAR and the RJ107 CAR were effective at specific lysis of the tumor cells.

### Example 4-In Vitro Activity of CARs Against Ewing's Sarcoma Cells

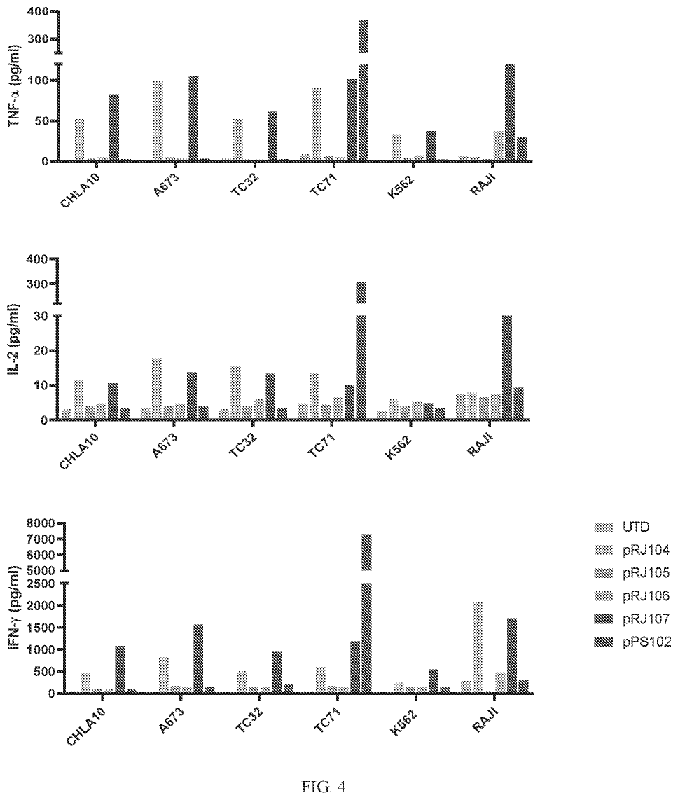

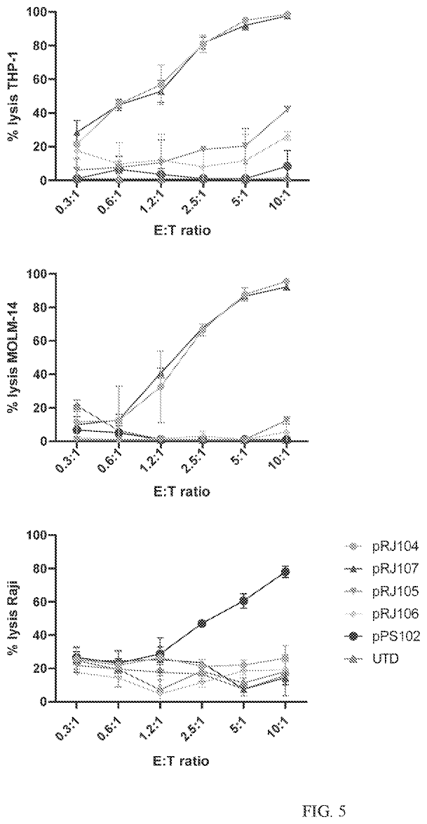

CAR-T cells specific for IL1RAP (RJ104, RJ105, RJ106, and RJ107), control CAR-T cells (UTD, untransduced), and CAR-T cells specific for CD19 (PS102) were cultured overnight with either Ewing sarcoma tumor cell lines expressing IL1RAP including CHLA10, A673, TC32, and TC71 cells; K562 negative control cells; or Raji cells which is an EBV(+)ve Burkitt lymphoma cell line. The TC71 cells were transduced to overexpress CD19. Cytokine production of the cultured cells was measured by ELISA using cell-free culture supernatants. As shown in FIG. 4, CAR T cells containing the RJ104 and RJ107 CARs and cultured with the Ewing sarcoma cell lines produced higher levels of TNF-alpha, IL-2, and Interferon-gamma. The highest levels of cytokine production were observed with TC71 cells co-cultured with CAR T cells containing CAR specific for CD19 which was consistent with overexpression of CD19 in the TC71 cells.

### Example 5 In Vitro Activity of CARs Against AML Cells

In vitro activity of CARs against acute myeloid leukemia (AML) cells was investigated. Effector CAR-T cells specific for IL1RAP were co-cultured with target AML cells at various ratios, and levels of cell lysis were measured. AML target cell lines included THP-1 or MOLM-14 cells. Raji cells, which were CD19+, were used as a control. As shown in FIG. 5, the IL1RAP-specific cell lines RJ104 and RJ107 had relatively high cytotoxic activity against the AML cell lines, while RJ105 and RJ106 had relatively low cytotoxic activity. The PS102 CAR-T effector was specific for the CD19 antigen and did not react with THP-1 or MOLM-14 AML lines, while it did recognize the CD19-positive cell line Raji. Thus, RJ104 and RJ107 showed high specific killing of AML.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and embodiments coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1.-166. (canceled)

167. A polypeptide capable of specifically binding an interleukin-1 receptor accessory protein (IL1RAP), comprising:
(i) a heavy chain variable domain or region comprising the amino acid sequences set forth in SEQ ID NO:196 (or SEQ ID NO:196 with one, two, or three amino acid additions, deletions, or substitutions), SEQ ID NO:197 (or SEQ ID NO:197 with one, two, or three amino acid additions, deletions, or substitutions), and SEQ ID NO: 198 (or SEQ ID NO:198 with one, two, or three amino acid additions, deletions, or substitutions);
(ii) a heavy chain variable domain or region comprising the amino acid sequences set forth in SEQ ID NO:203 (or SEQ ID NO:203 with one, two, or three amino acid additions, deletions, or substitutions), SEQ ID NO:204 (or SEQ ID NO:204 with one, two, or three amino acid additions, deletions, or substitutions), and SEQ ID NO:205 (or SEQ ID NO:205 with one, two, or three amino acid additions, deletions, or substitutions);
(iii) a heavy chain variable domain or region comprising the amino acid sequences set forth in SEQ ID NO:210 (or SEQ ID NO:210 with one, two, or three amino acid additions, deletions, or substitutions), SEQ ID NO:211 (or SEQ ID NO:211 with one, two, or three amino acid additions, deletions, or substitutions), and SEQ ID NO:212 (or SEQ ID NO:212 with one, two, or three amino acid additions, deletions, or substitutions);
(iv) a heavy chain variable domain or region comprising the amino acid sequences set forth in SEQ ID NO:217 (or SEQ ID NO:217 with one, two, or three amino acid additions, deletions, or substitutions), SEQ ID NO:218 (or SEQ ID NO:218 with one, two, or three amino acid additions, deletions, or substitutions), and SEQ ID NO:219 (or SEQ ID NO:219 with one, two, or three amino acid additions, deletions, or substitutions);
(v) a heavy chain variable domain or region comprising the amino acid sequences set forth in SEQ ID NO:161 (or SEQ ID NO:161 with one, two, or three amino acid additions, deletions, or substitutions), SEQ ID NO:162 (or SEQ ID NO:162 with one, two, or three amino acid additions, deletions, or substitutions), and SEQ ID NO: 163 (or SEQ ID NO:163 with one, two, or three amino acid additions, deletions, or substitutions);
(vi) a heavy chain variable domain or region comprising the amino acid sequences set forth in SEQ ID NO: 169 (or SEQ ID NO:169 with one, two, or three amino acid additions, deletions, or substitutions), SEQ ID NO:170 (or SEQ ID NO:170 with one, two, or three amino acid additions, deletions, or substitutions), and SEQ ID NO: 171 (or SEQ ID NO:171 with one, two, or three amino acid additions, deletions, or substitutions);
(vii) a heavy chain variable domain or region comprising the amino acid sequences set forth in SEQ ID NO: 177 (or SEQ ID NO:177 with one, two, or three amino acid additions, deletions, or substitutions), SEQ ID NO:178 (or SEQ ID NO:178 with one, two, or three amino acid additions, deletions, or substitutions), and SEQ ID NO:179 (or SEQ ID NO:179 with one, two, or three amino acid additions, deletions, or substitutions); or
(viii) a heavy chain variable domain or region comprising the amino acid sequences set forth in SEQ ID NO: 185 (or SEQ ID NO:185 with one, two, or three amino acid additions, deletions, or substitutions), SEQ ID NO:186 (or SEQ ID NO:186 with one, two, or three amino acid additions, deletions, or substitutions), and SEQ ID NO:187 (or SEQ ID NO:187 with one, two, or three amino acid additions, deletions, or substitutions).

168. The polypeptide of claim 167, wherein the heavy chain variable domain or region comprises an amino acid sequence having at least 90% sequence identity to the amino acid sequence set forth in any one of SEQ ID NOs:01, 02, 03, 04, 160, 168, 176, and 184.

169. A nucleic acid encoding the polypeptide of claim 167.

170. A chimeric antigen receptor (CAR) capable of specifically binding to an interleukin-1 receptor accessory protein (IL1RAP), comprising:
a ligand binding domain comprising the polypeptide of claim 167;
a polypeptide spacer;
a transmembrane domain; and
an intracellular signaling domain.

171. The CAR of claim 170, wherein the polypeptide spacer comprises an IgG1 hinge, an IgG2 hinge, an IgG4 hinge, a CD8 hinge, a CD28 hinge, or the amino acid sequence of any one of SEQ ID NOs:72-94 and 115-123.

172. The CAR of claim 170, wherein the transmembrane domain comprises a transmembrane domain derived from CD37, CD4, CD8α, CD28, CD278, or the amino acid sequence of any one of SEQ ID NOs:13, and 124-131.

173. The CAR of claim 170, wherein the intracellular signalling domain comprises a costimulatory domain selected from the group consisting of CD27, CD28, 4-1BB, OX-40, CD30, CD40, PD-1, ICOS, LFA-1, CD2, CD7, NKG2C, and B7-H3, in combination with a CD3zeta domain or functional portion thereof, or the amino acid sequence of any one of SEQ ID NOs:15, 17, and 132-139.

174. A polynucleotide encoding the CAR of claim 170.

175. A vector comprising the polynucleotide of claim 174.

176. The vector of claim 175, wherein the vector is a viral vector.

177. A cell comprising the CAR of claim 170.

178. The cell of claim 177, wherein said cell is a T cell, a stem cell, an NK cell, a precursor T cell, or a hematopoietic stem cell.

179. A pharmaceutical composition comprising the cell of claim 177 and a pharmaceutically acceptable excipient.

180. A method of treating, ameliorating or inhibiting an IL1RAP⁺ cancer in a subject, comprising administering the cell of claim 177 to the subject.

181. The method of claim 180, wherein the cancer is selected from the group consisting of a breast cancer, a brain cancer, a colon cancer, a renal cancer, a pancreatic cancer, an ovarian cancer, a sarcoma, a leukemia, an acute myeloid leukemia (AML), a chronic myelogenous leukemia (CML), or a Ewing's sarcoma.

182. A cell engager comprising a first antigen binding domain comprising the polypeptide of claim 167, a linker, and a second antigen binding domain.

183. The cell engager of claim 182, wherein: the linker comprises an IgG1 hinge, an IgG2 hinge, an IgG4 hinge, a CD8 hinge, a CD28 hinge, or the amino acid sequence of any one of SEQ ID NOs:72-94 and 115-123;
the second antigen binding domain specifically binds to CD3, CD16α, NKG2A, NKG2D, NKp30, NKp44, or NKp46;
the second antigen binding domain comprises the amino acid sequence of SEQ ID NO:140-143, and 144-149; and/or
further comprises a third antigen binding region.

184. An antibody-drug conjugate (ADC) comprising the polypeptide of claim 167.

185. The ADC of claim 184, wherein the drug is selected from the group consisting of an auristatin, mertansine, and a pyrrolobenzodiazepine (PBD) dimer.

186. A method of treating, ameliorating or inhibiting an IL1RAP⁺ cancer in a subject, comprising administering the ADC of claim 184 to the subject.
